SEQUENCE LISTING (17 sequences)

SEQ ID NO: 1 - DNA, 100 bp, Artificial Sequence
  source: /note=“Description of Artificial Sequence Synthetic polynucleotide”
acctcctcaaaccaacttccatttaaatacgattaaataatattaatatattatcgattaaataataattaattaatatttatggatggattgaggatgg

SEQ ID NO: 2 - DNA, 100 bp, Artificial Sequence
  source: /note=“Description of Artificial Sequence Synthetic polynucleotide”
ccatcctcaatccatccataaatattaattaattattatttaatcgataatatattaatattatttaatcgtatttaaatggaagttggtttgaggaggt

SEQ ID NO: 3 - DNA, 100 bp, Artificial Sequence
  source: /note=“Description of Artificial Sequence Synthetic polynucleotide”
ctcacccacaaccacaaacaatttaaatacgattaaataatattaatatattatcgattaaataataattaattaatattggttggatggtagatggtga

SEQ ID NO: 4 - DNA, 100 bp, Artificial Sequence
  source: /note=“Description of Artificial Sequence Synthetic polynucleotide”
tcaccatctaccatccaaccaatattaattaattattatttaatcgataatatattaatattatttaatcgtatttaaattgtttgtggttgtgggtgag

SEQ ID NO: 5 - DNA, 100 bp, Artificial Sequence
  source: /note=“Description of Artificial Sequence Synthetic polynucleotide”
ttcctcatattccctccctcatttaaatacgattaaataatattaatatattatcgattaaataataattaattaatatttgatgtgatgggtggtatgg

SEQ ID NO: 6 - DNA, 100 bp, Artificial Sequence
  source: /note=“Description of Artificial Sequence Synthetic polynucleotide”
ccataccacccatcacatcaaatattaattaattattatttaatcgataatatattaatattatttaatcgtatttaaatgagggagggaatatgaggaa

SEQ ID NO: 7 - DNA, 100 bp, Artificial Sequence
  source: /note=“Description of Artificial Sequence Synthetic polynucleotide”
atccacatcattacccatccatttaaatacgattaacgaacattaatacgttatcgattaaataagacgtaattaatatttgatgtgatgggtggtatgg

SEQ ID NO: 8 - DNA, 100 bp, Artificial Sequence
  source: /note=“Description of Artificial Sequence Synthetic polynucleotide”
ccataccacccatcacatcaaatattaattacgtcttatttaatcgataacgtattaatgttcgttaatcgtatttaaatggatgggtaatgatgtggat

SEQ ID NO: 9 - DNA, 20 bp, Artificial Sequence
  source: /note=“Description of Artificial Sequence Synthetic primer”
acctcctcaaaccaacttcc

SEQ ID NO: 10 - DNA, 20 bp, Artificial Sequence
  source: /note=“Description of Artificial Sequence Synthetic primer”
ccatcctcaatccatccata

SEQ ID NO: 11 - DNA, 20 bp, Artificial Sequence
  source: /note=“Description of Artificial Sequence Synthetic primer”
ctcacccacaaccacaaaca

SEQ ID NO: 12 - DNA, 20 bp, Artificial Sequence
  source: /note=“Description of Artificial Sequence Synthetic primer”
tcaccatctaccatccaacc

SEQ ID NO: 13 - DNA, 20 bp, Artificial Sequence
  source: /note=“Description of Artificial Sequence Synthetic primer”
ttcctcatattccctccctc

SEQ ID NO: 14 - DNA, 20 bp, Artificial Sequence
  source: /note=“Description of Artificial Sequence Synthetic primer”
ccataccacccatcacatca

SEQ ID NO: 15 - DNA, 20 bp, Artificial Sequence
  source: /note=“Description of Artificial Sequence Synthetic primer”
atccacatcattacccatcc

SEQ ID NO: 16 - DNA, 20 bp, Artificial Sequence
  source: /note=“Description of Artificial Sequence Synthetic primer”
ccataccacccatcacatca

SEQ ID NO: 17 - protein, 8 aa, Artificial Sequence
  source: /note=“Description of Artificial Sequence Synthetic peptide”
DYKDDDDK

(19) United States
(12) Patent Application Publication
(10) Pub. No.: US 2023/0102739 A1
(43) Pub. Date: Mar. 30, 2023

(54) DETECTION OF EPIGENETIC MODIFICATIONS

(71) Applicant: Cambridge Epigenetix Limited, Saffron Walden (GB)

(72) Inventors: Michael Steward, Royston (GB); Tobias Ost, Wilburton (GB); Shirong Yu, Cambridge (GB); Helen Bignell, Cambridge (GB)

(21) Appl. No.: 16/614,097

(22) PCT Filed: May 15, 2018
(86) PCT No.: PCT/IB2018/000680

§ 371 Date: Nov. 15, 2019

Related U.S. Application Data
(60) Provisional application No. 62/638,528, filed on Mar. 5, 2018.
(60) Provisional application No. 62/507,035, filed on May 16, 2017.

(51) Int. Cl. C12Q 1/6827; C12Q 1/6858

(52) U.S. Cl. CPC C12Q 1/6827; C12Q 1/6858

(57) ABSTRACT

Provided herein are systems and methods for detection of an epigenetic modification in a nucleic acid sequence. The systems and methods as described herein may provide a substantially unbiased approach in detecting an epigenetic modification. The systems and method as described herein may provide a substantially unbiased approach in detecting an epigenetic modification in comparison to systems and methods that amplify sequences having a label or a moiety associated with an epigenetic modification.

### CROSS-REFERENCE

This application claims the benefit of U.S. provisional application 62/507,035 filed on May 16, 2017, and. U.S. Provisional Patent Application No. 62/638, 528 filed on Mar. 5, 2018, which are herein incorporated by reference in their entireties.

### BACKGROUND

It is important to develop new methods to determine methylation status and to monitor changes in methylation status.

### SUMMARY

The systems and methods as described herein may provide a substantially unbiased approach in detecting an epigenetic modification. This method may be an improvement in the field of detecting or monitoring methylation status particularly when compared to systems and methods that amplify sequences having a label or a moiety associated with an epigenetic modification.

### INCORPORATION BY REFERENCE

All publications, patents, and patent applications herein are incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede or take precedence over any such contradictory material.

### BRIEF DESCRIPTION OF THE DRAWINGS

The novel features herein are set forth with particularity in the appended claims. A better understanding of the features and advantages herein will be obtained by reference to the following detailed description that sets forth illustrative embodiments, in which the principles herein are utilized, and the accompanying drawings (also “figure” and “FIG.” herein), of which:

FIG. 1 shows a computer control system that may be programmed or otherwise configured to implement methods provided herein.

FIG. 2 shows one example of the 5-hydroxymethylcytosine (5-hmC) Pulldown Label Copy Enrich (HMCP_LCE) method detailed herein.

FIG. 3 shows one example of the 5-hmC Pulldown Copy Label Enrich (HMCP_CLE) method detailed herein.

FIG. 4 shows one example of the 5-hmC Pulldown Label Random prime Enrich (HMCP_LRE) method detailed herein.

FIG. 5 shows one example of the 5-hmC Pulldown Random primer Label Enrich (HMCP_RLE) method detailed herein.

FIG. 6 shows one example of the 5-hmC Pulldown Label Loci Specific Enrich (HMCP_LLSE) method detailed herein.

FIG. 7 shows one example of the 5-hmC Pulldown Loci Specific Label Enrich (HMCP_LSLE) method detailed herein.

FIG. 8A-FIG. 8B shows a band shift assay of 6 5-hmC using different T4 Phage beta-glucosyltransferase (T4-BGT) buffers.

FIG. 9 shows a comparison of detailed sequencing metrics between the Copy Label Enrich (CLE) method and the 5-hmC pulldown (HMCP) method (also referred to as the ‘standard HMCP’ or ‘std’).

FIG. 10 shows an Integrative Genomics Viewer (IGV) screenshot of an 18 kilobase (kB) region of the human genome and the alignment of pulled-down reads.

FIG. 11 shows an IGV screenshot of a genomic region.

FIG. 12 shows an IGV screenshot of a region of the human genome with dense CpGs.

FIG. 13A-FIG. 13B shows summary metrics from the Cope Label Enrich (CLE) method in comparison to the 5-hmC pulldown (HMCP) method.

FIG. 14 shows SEQ. ID. NO. 1-8.

FIG. 15 shows SEQ. ID. NO. 9-16.

FIG. 16 shows a diagram of the methods and systems as disclosed herein.

FIG. 17 shows a comparison between the methods of HMCP (shown as “HMCP” on the x-axis) and CLE (shown as “CLE-HMCP” on the x-axis) of an enrichment ratio of 6 5-hmC/2 5-hmC by quantitative polymerase chain reaction (qPCR).

FIG. 18 shows a comparison between the methods of HMCP (shown as “HMCP” in the figure legend) and CLE (shown as “CLE_HMCP” in the figure legend) of a ratio of reads that map to inside genebodies as compared to those reads that map to intergenic regions.

FIG. 19 shows a comparison between the HMCP (shown as “v1HMCP”) and CLE (shown as “v2HMCP”) methods of a percentage of the genome covered.

FIG. 20 shows an IGV screenshot comparing HMCP (shown as “V1”) and CLE (shown as “V2”) methods on whole genomic DNA (wgDNA) extracted from normal colon tissue and colon tumour tissue at the beta- actin (ACTB) locus. A normal sample analyzed by the HMCP method is labeled “V1 Normal.” A normal sample analyzed by the CLE method is labeled “V2 Normal.” A tumour sample analyzed by the HMCP method is labeled “V1 Tumour.” A tumour sample analyzed by the CLE method is labeled “V2 Tumour.”

FIG. 21 shows an IGV screenshot comparing HMCP (shown as “V1”) and CLE (shown as “V2”) methods on whole genomic DNA (wgDNA) extracted from normal colon tissue and colon tumour tissue at the start of the NaCC2 locus. A normal sample analyzed by the HMCP method is labeled “V1 Normal.” A normal sample analyzed by the CLE method is labeled “V2 Normal.” A tumour sample analyzed by the HMCP method is labeled “V1 Tumour.” A tumour sample analyzed by the CLE method is labeled “V2 Tumour.”

FIG. 22 shows an IGV screenshot comparing HMCP (shown as “V1”) and CLE (shown as “V2”) methods on whole genomic DNA (wgDNA) extracted from normal colon tissue and colon tumour tissue of DLL1 gene and adjacent loci on chromosome 6. A normal sample analyzed by the HMCP method is labeled “V1 Normal.” A normal sample analyzed by the CLE method is labeled “V2 Normal.” A tumour sample analyzed by the HMCP method is labeled “V1 Tumour.” A tumour sample analyzed by the CLE method is labeled “V2 Tumour.”

FIG. 23 shows an IGV screenshot comparing HMCP (shown as “V1”) and CLE (shown as “V2”) methods on whole genomic DNA (wgDNA) extracted from normal colon tissue and colon tumour tissue of a region on chromosome 9. A normal sample analyzed by the HMCP method is labeled “V1 Normal.” A normal sample analyzed by the CLE method is labeled “V2 Normal.” A tumour sample analyzed by the HMCP method is labeled “V1 Tumour.” A tumour sample analyzed by the CLE method is labeled “V2 Tumour.”

FIG. 24 shows an IGV screenshot comparing HMCP (shown as “V1”) and CLE (shown as “V2”) methods on whole genomic DNA (wgDNA) extracted from normal colon tissue and colon tumour tissue of a region of Chr9 with sparse CpG distribution. A normal sample analyzed by the HMCP method is labeled “V1 Normal.” A normal sample analyzed by the CLE method is labeled “V2 Normal.” A tumour sample analyzed by the HMCP method is labeled “V1 Tumour.” A tumour sample analyzed by the CLE method is labeled “V2 Tumour.”

FIG. 25 shows an IGV screenshot comparing HMCP (shown as “V1”) and CLE (shown as “V2”) methods on whole genomic DNA (wgDNA) extracted from normal colon tissue and colon tumour tissue of a 785 base pair (bp) region of human Chr17 gene where there is a large gap between CpG islands. A normal sample analyzed by the HMCP method is labeled “V1 Normal.” A normal sample analyzed by the CLE method is labeled “V2 Normal.” A tumour sample analyzed by the HMCP method is labeled “V1 Tumour.” A tumour sample analyzed by the CLE method is labeled “V2 Tumour.”

FIG. 26 shows an IGV screenshot of brain-specific 5-hmC peaks that can be detected in the context of the NA12878 derived peaks at levels as low as about 1% cerebellum.

FIG. 27 shows a scatterplot comparison of HMCP (shown as “HMCP-v1”) and CLE (shown as “HMCP-v2”) methods using plasma DNA.

FIG. 28 shows an IGV screenshot highlighting a correlation between HMCP and TrueMethyl Whole Genome (TMWG) across different CpG densities.

FIG. 29 shows a comparison of reads per kilobase per million mapped reads (RPKM) values on a heatmap between HMCP (shown as “HMCP-v1”) and CLE (shown as “HMCP-v2”) methods.

FIG. 30 shows a multidimensional scaling (MDS) plot showing a level of similarity of read counts over genebodies for samples from a titration of cerebellum tissue genomic DNA (gDNA) into a background of NA12878 peripheral blood mononuclear cell (PBMC) cell line gDNA.

FIG. 31A shows a Q-Q plot using the CLE (shown as “HMCP-v2”) method of TMWG % 5-hmC (25.01-99.99%) and HMCP genebodies RPKM.

FIG. 31B shows a Q-Q plot using the CLE (shown as “HMCP-v2”) method of TMWG % 5-hmC (25.01-99.99%) and HMCP genebodies RPKM.

FIG. 31C shows a Q-Q plot using the HMCP (shown as “HMCP-v1”) method of TMWG % 5-hmC (25.01-99.99%) and HMCP genebodies RPKM.

FIG. 32A shows a sequence read enrichment for the HMCP method.

FIG. 32B shows a sequence read enrichment for the CLE method. The CLE method shows higher pulldown efficiency as compared with the HMCP method shown in FIG. 32A.

FIG. 33 shows an MDS plot for 3311 functional regions of the human genome.

### DETAILED DESCRIPTION

While various embodiments have been shown and described herein, it will be obvious to those skilled in the art that such embodiments are provided by way of example only. It should be understood that various alternatives to the embodiments herein may be employed.

### Overview

A method as described herein may comprise associating a label with an epigenetically modified base of a nucleic acid sequence to form a labeled nucleic acid sequence; hybridizing a substantially complementary strand to the labeled nucleic acid sequence; and amplifying the substantially complementary strand in a reaction in which the labeled nucleic acid sequence is substantially not present. One or more individual elements of the method need not be performed in a particular order. For example, associating a label may occur after the hybridizing. One or more individual elements of a given method may be performed in a different order than described herein.

### Method—Variation 1

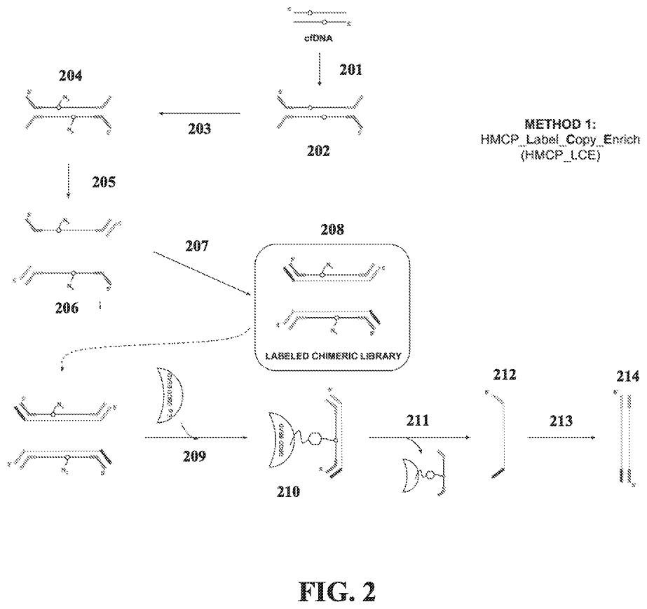

FIG. 2 shows one example of the 5-hmC Pulldown Label Copy Enrich (HMCP_LCE) method detailed herein. Advantages of the HMCP_LCE method may provide: (a) an improved resolution as compared to other methods, such as a HMCP method or a method that may associate a sugar, an antibody, a protein, a fragment of any of these, a label, or any combination thereof with an epigenetically modified base of the nucleic acid; (b) a decrease in 5-hmC-density bias as compared to other methods, such as a HMCP method or a method that may associate a sugar, an antibody, a protein, a fragment of any of these, a label, or any combination thereof with an epigenetically modified base of the nucleic acid; or (c) any combination thereof.

In this example of FIG. 2, a first element 201 may be to prepare a plurality of double-stranded fragments 202, such as a library of oligonucleotide fragments. The plurality of double-stranded fragments may comprise cell-free DNA. The plurality of double-stranded fragments may comprise one or more epigenetic modifications on one or both strands. A second element 203 may be to associate a label (such as an azido-glucose label) with at least one of the oligonucleotide fragments from the plurality of double-stranded fragments to form a modified oligonucleotide fragment 204. The label may associate with an epigenetic modification present at one or more bases of the modified oligonucleotide fragment. A third element 205 may be to separate the modified oligonucleotide fragment to form one or more single-stranded modified oligonucleotide fragments 206. A fourth element 207 may be to hybridize a complementary strand, such as a substantially complementary strand, to a single-stranded modified oligonucleotide fragment to form a modified oligonucleotide fragment 208, such as a labeled chimeric library. The complementary strand may lack one or both of the label and the epigenetic modification. A fifth element 210 may be to associate a label 209 with the modified oligonucleotide fragment wherein the label 209 may also associate with a substrate. The label 209 may bind to an epigenetic modification or to a label previously associated with an epigenetic modification. The label 209 may not bind directly to the complementary strand. The complementary strand may be indirectly associated with the substrate via the interaction between the substrate and the modified oligonucleotide fragment. The association between the complementary strand and the opposing strand may be disruptable, such as a disruptable bond. A sixth element 211 may be to enrich a sample for one or more complementary strands 212 by removing or separating or washing away from the substrate one or more complementary strands (such as by disrupting the bond between the complementary strand and the opposing strand) and then separating the complementary strand from the modified oligonucleotide fragment that remains associated with the substrate. A seventh element 213 may be to amplify the enriched complementary strand in the absence of the modified oligonucleotide fragment to form one or more daughter strands 214 of the complementary strand.

In FIG. 2, the library may comprise double-stranded oligonucleotide fragments or single-stranded oligonucleotide fragments. The oligonucleotide fragments may be DNA or RNA. The library may be a next-generation (NGS) library. The library may comprise an oligonucleotide fragment having an adaptor (such as an NGS adaptor) at (a) one or both ends of the fragment, (b) at one or both strands of the double-stranded oligonucleotide fragment, or (c) a combination thereof. The adaptor may uniquely identify the oligonucleotide fragment from other oligonucleotide fragments in a sample or in a library. The adaptor may be specific to or selective for double-stranded DNA.

In FIG. 2, a label may associate with an epigenetic modification (such as 5-hmC) or a type of epigenetic modification present at a base of the oligonucleotide fragment. A label may associate with a plurality of epigenetic modifications present on one or both strands of a double-stranded oligonucleotide fragment. A label may associate with a type of epigenetic modification (such as 5-hmC). A label may be selective for a type of epigenetic modification (such as a 5-hmC). The label may be selective for double-stranded oligonucleotide fragments and may not label single-stranded fragments. The label may be selective for single-stranded oligonucleotide fragments. The label may associate with (such as bind to) the epigenetic modification with an aid, such as an enzyme. The enzyme may be selective for double-stranded oligonucleotide fragments, such as beta-glucosyltransferase (bGT). The label may associate with the epigenetic modification by click chemistry. The label may be an azido-sugar, such as an azido-glucose.

In FIG. 2, a double-stranded oligonucleotide fragment may be separated to form single stranded fragments, such as separating by denaturation. A complementary strand may be hybridized to at least a portion of a single stranded oligonucleotide. A complementary strand may be a primer, such as a primer that may be complementary to the adaptor (such as an NGS adaptor). A complementary strand may be a substantially complementary strand, such as substantially complementary along an entire length of the oligonucleotide fragment. The substantially complementary strand may be absent (a) the label that may be present in the parent oligonucleotide fragment, (b) the epigenetic modification that may be present in the parent oligonucleotide fragment, or (c) a combination thereof. The substantially complementary strand may be hybridized to the parent oligonucleotide fragment by DNA extension or cDNA extension.

In FIG. 2, parent oligonucleotide fragments and the substantially complementary strand may be indirectly associated with a substrate. The association to the substrate may occur via the label associated with the epigenetic modification on the parent oligonucleotide fragment. The substantially complementary strand may be free of any label and/or free of any epigenetic modification. The association between the label and the substrate may be disrupted.

In FIG. 2, oligonucleotide fragments comprising an epigenetic modification may be separated from oligonucleotide fragments absent any epigenetic modifications or absent a type of epigenetic modification. Separation may occur by associating the label with a substrate, such that any fragment absent the epigenetic modification or the type of epigenetic modification may be removed. Removal may occur by washing, such as stringent washing of the substrate. Following removal of oligonucleotide fragments lacking an epigenetic modification or a type of epigenetic modification, the substantially complementary strand may be separated from the parent oligonucleotide fragment strand. The parent oligonucleotide fragment strand may remain associated with the substrate. The parent oligonucleotide fragment strand and the substrate may be discarded. The substantially complementary strand may be amplified in a reaction vessel that may be free of the parent oligonucleotide fragment strand.

### Method—Variation 2

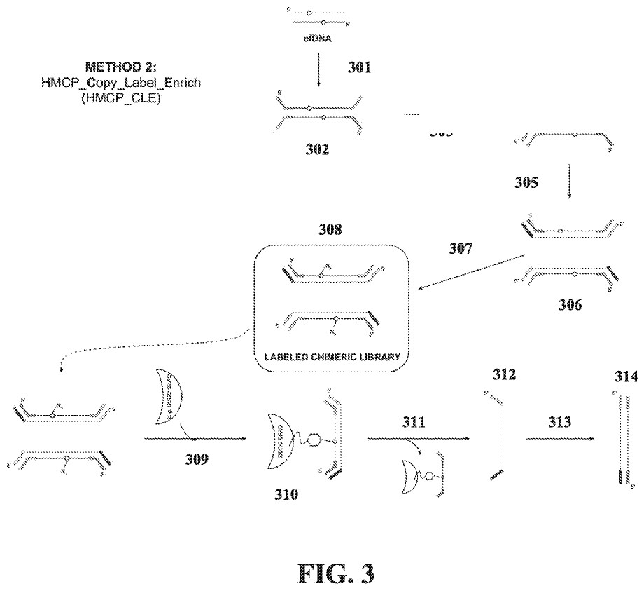

FIG. 3 shows one example of the 5-hmC Pulldown Copy Label Enrich (HMCP_CLE) method detailed herein. In some cases, the HMCP_CLE method may provide: (a) an improved resolution as compared to other methods, such as a HMCP method or a method that may associate a sugar, an antibody, a protein, a fragment of any of these, a label, or any combination thereof with an epigenetically modified base of the nucleic acid; (b) a decrease in 5-hmC-density bias as compared to other methods, such as a HMCP method or a method that may associate a sugar, an antibody, a protein, a fragment of any of these, a label, or any combination thereof with an epigenetically modified base of the nucleic acid; or (c) any combination thereof.

In this example of FIG. 3, a first element 301 may be to prepare a plurality of double stranded oligonucleotide fragments 302, such as a library. The double stranded oligonucleotide fragments may comprise cell-free DNA. The double stranded oligonucleotide fragments may have epigenetic modifications on one or more bases of one or both strands. A second element 303 may be to separate the strands of a double-stranded oligonucleotide fragment of the plurality to form one or more single-stranded oligonucleotide fragments 304. The one or more single-stranded oligonucleotide fragments may comprise one or more bases having an epigenetic modification. A third element 305 may be to hybridize a complementary strand, such as a substantially complementary strand, to at least one single-stranded oligonucleotide fragment to form a modified oligonucleotide fragment 306. The complementary strand may be substantially free of the epigenetic modification present in the opposing single-stranded oligonucleotide fragment. A fourth element 307 may be to associate a label (such as an azido-glucose label) with the modified oligonucleotide fragment to form a labeled modified oligonucleotide fragment 308, such as a labeled chimeric library. The label may associate with an epigenetic modification present in the modified oligonucleotide fragment. The label may not be associated with the substantially complementary strand that may lack an epigenetic modification. A fifth element 310 may be to associate a label 309 with the modified oligonucleotide fragment wherein the label 309 may also associate with a substrate. The label 309 may not bind directly to the complementary strand. The complementary strand may be indirectly associated with the substrate via the interaction between the substrate and the modified oligonucleotide fragment. The association between the complementary strand and the opposing strand may be disruptable, such as a disruptable bond. A sixth element 311 may be to enrich a sample for one or more complementary strands 312 by removing or separating or washing away from the substrate one or more complementary strands (such as by disrupting the bond between the complementary strand and the opposing strand). Upon separation, the modified oligonucleotide fragment may remain associated with the substrate. In some cases, enriching a sample for one or more complementary strands may comprise washing a substrate, such as stringent washing of a substrate. Washing may remove one or more non-covalently bound fragments, one or more non-specifically physisorbed fragments, or a combination thereof. Washing may not disrupt or alter an association between a modified oligonucleotide fragment and a substrate, such that a sample may be enriched for the complementary strand. A seventh element 313 may be to amplify the complementary strand in the absence of the modified oligonucleotide fragment to form one or more daughter strands 314 of the complementary strand.

In FIG. 3, the library may comprise double-stranded oligonucleotide fragments or single-stranded oligonucleotide fragments. The oligonucleotide fragments may be DNA or RNA. The library may be a next-generation (NGS) library. The library may comprise an oligonucleotide fragment having an adaptor (such as an NGS adaptor) at (a) one or both ends of the fragment, (b) at one or both strands of the double-stranded oligonucleotide fragment, or (c) a combination thereof. The adaptor may uniquely identify the oligonucleotide fragment from other oligonucleotide fragments in a sample or in a library. The adaptor may be specific to or selective for double-stranded DNA.

In FIG. 3, a double-stranded oligonucleotide fragment may be separated to form single stranded fragments, such as separating by denaturation. A complementary strand may be hybridized to at least a portion of a single stranded oligonucleotide. A complementary strand may be a primer, such as a primer that may be complementary to the adaptor (such as an NGS adaptor). A complementary strand may be a substantially complementary strand, such as substantially complementary along an entire length of the oligonucleotide fragment. The substantially complementary strand may be absent the epigenetic modification that may be present in the parent oligonucleotide fragment. The substantially complementary strand may be hybridized to the parent oligonucleotide fragment by cDNA extension.

In FIG. 3, a label may associate with an epigenetic modification (such as 5-hmC) or a type of epigenetic modification present at a base of the parent oligonucleotide fragment. A label may associate with a plurality of epigenetic modifications present on the parent oligonucleotide fragment. A label may associate with a type of epigenetic modification (such as 5-hmC). A label may be selective for a type of epigenetic modification (such as a 5-hmC). The label may be selective for double-stranded fragments and may not label single-stranded fragments. The label may be selective for single-stranded fragments. The label may associate with (such as bind to) the epigenetic modification of the parent strand with an aid, such as an enzyme. The enzyme may be selective for double-stranded oligonucleotide fragments, such as beta-glucosyltransferase (bGT). The label may associate with the epigenetic modification by click chemistry. The label may be an azido-sugar, such as an azido-glucose.

In FIG. 3, parent oligonucleotide fragments and the substantially complementary strand may be indirectly associated with a substrate. The association to the substrate may occur via the label associated with the epigenetic modification on the parent oligonucleotide fragment. The substantially complementary strand may be free of any label and/or free of any epigenetic modification. The association between the label and the substrate may be disrupted.

In FIG. 3, oligonucleotide fragments comprising an epigenetic modification may be separated from oligonucleotide fragments absent any epigenetic modifications or absent a type of epigenetic modification. Separation may occur by associating the label with a substrate, such that any fragment absent the epigenetic modification or the type of epigenetic modification may be removed. Removal may occur by washing, such as stringent washing of the substrate. Following removal of oligonucleotide fragments lacking an epigenetic modification or a type of epigenetic modification, the substantially complementary strand may be separated from the parent oligonucleotide fragment strand. The parent oligonucleotide fragment strand may remain associated with the substrate. The parent oligonucleotide fragment strand and the substrate may be discarded. The substantially complementary strand may be amplified in a reaction vessel that may be free of the parent oligonucleotide fragment strand.

### Method—Variation 3

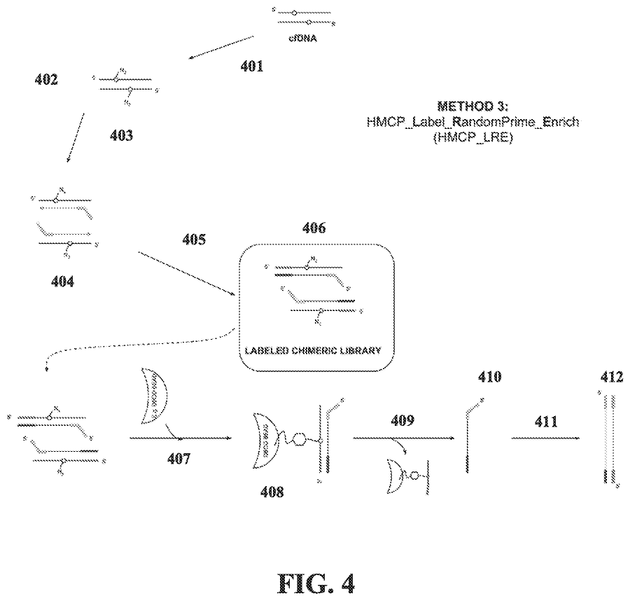

FIG. 4 shows one example of the 5-hmC Pulldown Label Random prime Enrich (HMCP_LRE) method detailed herein. In some cases, the HMCP_LRE method may provide: (a) an improved resolution as compared to other methods, such as a HMCP method or a method that may associate a sugar, an antibody, a protein, a fragment of any of these, a label, or any combination thereof with an epigenetically modified base of the nucleic acid; (b) a decrease in 5-hmC-density bias as compared to other methods, such as a HMCP method or a method that may associate a sugar, an antibody, a protein, a fragment of any of these, a label, or any combination thereof with an epigenetically modified base of the nucleic acid; (c) a substantially improved robustness at low input mass as compared to other methods, such as a HMCP method or a method that may associate a sugar, an antibody, a protein, a fragment of any of these, a label, or any combination thereof with an epigenetically modified base of the nucleic acid; or (d) any combination thereof.

In this example of FIG. 4, a first element 401 may be to associate a label (such as an azido-glucose label) with a double stranded oligonucleotide fragment to yield a modified oligonucleotide fragment 402. The double stranded oligonucleotide may comprise cell-free DNA. The label may associate with an epigenetic modification or a type of epigenetic modification present at a base of one or both strands of the double stranded oligonucleotide fragment to form the modified oligonucleotide fragment 402. A second element 403 may be to separate the strands of the modified oligonucleotide fragment to form one or more single-stranded modified oligonucleotide fragments and then to hybridize a complementary strand, such as a substantially complementary strand to at least one of the single-stranded modified oligonucleotide fragments to form a double stranded modified oligonucleotide fragment 404 having a complementary strand and a modified oligonucleotide fragment having the label. The complementary strand may be absent the label and absent the epigenetic modification. A third element 405 may associate an adaptor to the double stranded modified oligonucleotide fragment (such as to one or both ends of one or both strands of the double stranded modified oligonucleotide fragment) to form a double stranded modified oligonucleotide fragment having one or more adaptors 406, such as a labeled chimeric library. A fourth element 408 may be to associate a label 407 with the modified oligonucleotide fragment wherein the label 407 may also associate with a substrate. The label 408 may bind to an epigenetic modification or to the label previously associated with an epigenetic modification. The label 408 may not bind directly to the complementary strand. The complementary strand may be indirectly associated with the substrate via the interaction between the substrate and the modified oligonucleotide fragment. The interaction between the complementary strand and the opposing strand may be disruptable, such as a disruptable bond. A fifth element 409 may be to enrich a sample for one or more complementary strands 410 by removing or separating or washing away from the substrate one or more complementary strands that lack a label associated with the substrate (such as by disrupting the bond between the complementary strand and the opposing strand) and then separating the complementary strand from the modified oligonucleotide fragment that remains associated with the substrate. A sixth element 411 may be to amplify the complementary strand in the absence of the modified oligonucleotide fragment to form one or more daughter strands 412 of the complementary strand.

In FIG. 4, a label may associate with an epigenetic modification (such as 5-hmC) present at a base of the parent oligonucleotide fragment. A label may associate with a plurality of epigenetic modifications present on the parent oligonucleotide fragment. A label may associate with a type of epigenetic modification (such as 5-hmC). A label may be selective for a type of epigenetic modification (such as a 5-hmC). The label may be selective for double-stranded fragments and may not label single-stranded fragments. The label may be selective for single-stranded fragments. The label may associate with (such as bind to) the epigenetic modification of the parent strand with an aid, such as an enzyme. The enzyme may be selective for double-stranded oligonucleotide fragments, such as beta-glucosyltransferase (bGT). The label may associate with the epigenetic modification by click chemistry. The label may be an azido-sugar, such as an azido-glucose.

In FIG. 4, a position of a label may be determined by the presence/absence of 5-hmC in a dsDNA parent fragment. A label may be an azido-glucose, transferred to a 5-hmC from UDP-6-azide-glucose (UDP-N3-glc) by beta-glucosyltransferase (βGT). Labeling may be performed directly on a purified circulating tumor DNA (ctDNA) extract. An advantage may be that a ctDNA may not have been through a series of library preps ahead of labeling. There may be likely more material at labeling (improved efficiency) and presenting a more representative sample to a labeling than may be the case post NGS prep.

In some cases, hybridizing may comprise (i) priming (such as random priming), (ii) ligation (such as adapter ligation), or (iii) a combination thereof. For example, in FIG. 4, random priming may be performed by incubating an azido-labeled double-stranded DNA (dsDNA) duplex in the presence of an oligomer pool (where each oligo in the pool may comprise a degenerate N6, N7, N8, N9, N10 or beyond “head” attached to a “NGS-adapter” tail), a DNA polymerase (e.g. Klenow) and a native nucleoside triphosphate comprising deoxyribose (dNTP) mix in a given buffer, and performing a single extension reaction at 37° C. for a defined time (e.g. 10 mins). A degenerate primer “head” randomly may prime a template DNA and may make multiple copies for each of the parent strands. If using a strand displacing polymerase, the random primer that primer closest to the 3′ end of the template strand may extend and displace the other copies, leading to a long, double stranded chimeric product with a 3′A-overhang at the end of the daughter copy. Random priming may achieve two elements in one by: 1) introducing an NGS-specific adapter sequence and 2) generating a modification-free copy (daughter strand) of the modified parent strand.

In FIG. 4, adapter ligation may occur by incubating a mono-adapted chimeric labelled duplex template with a NGS-platform specific adapter (a forked adapter, a linear duplex adapter, a hairpin adapter, or a combination thereof) with 3′ T overhang and 5′ PO4 end, a dsDNA ligase (e.g. T4 ligase) and necessary cofactors (e.g. Mg2+, adenosine triphosphate (ATP), polyethylene glycol (PEG)) in a given buffer, at 20° C. for a defined period of time (e.g. 15 minutes). The A overhang of the monoadapted chimeric labelled duplex may match with the T overhang of the adapter and may promote ligation efficiency. Only one end of each duplex (that being formed by the 3′ end of the daughter strand) may be adapted. A successful ligation product may have a singly adapted azido-labeled parent strand (5′adapted) and a doubly adapted non-modified daughter strand (both 3′ and 5′ ends). In some cases, amplification of such “library”, only a bottom strand may be amplifiable with an adapter-specific polymerase chain reaction (PCR) primer.

In FIG. 4, magnetic bead binding may enable selective enrichment of a labeled chimeric next generation sequencing (NGS) library fragments. This may be achieved directly (i.e. by Sharpless Azide-alkyne cycloaddition reaction (CLICK) chemistry between the azido-glucose label and dibenzocyclooctyne (DBCO)-magbead) or indirectly (i.e. by Sharpless Azide-alkyne cycloaddition reaction (CLICK) of a dibenzocyclooctyne (DBCO)-biotin linker and then conjugation of the product to streptavidin-magbeads). In some cases, only azido-labeled fragments (i.e. 5-hmC-containing) may bind to the magbead. Azido-labeled fragments may be immobilized to a bead, such as a magnetic bead. In some cases, this interaction may only occur via a labeled parent strand of the chimeric NGS library duplex. A copied complement may not be azido-labeled and thus may be immobilized to a bead by virtue of the hydrogen-bonding interaction between the complementary duplex strands. As this H-bonding interaction may be non-covalent, it may be disrupted and exploited in downstream steps.

In FIG. 4, enrichment by stringent washing may be essential to maximize a signal-to-noise ratio of an enrichment process. Chimeric NGS library immobilized beads may be washed stringently (e.g. specific buffers; mild heat; mild denaturants etc.) to selectively remove non-covalently bound NGS library fragments, non-specifically physiosorbed to their surface. In some cases, such types of fragments may cause noise in a final sequencing result. Chimeric NGS library fragments covalently bound to the bead surface may be selected for in the enrichment (i.e. signal, those whose may insert originally contained 5-hmC). After stringent washing, a daughter strand may be eluted from the bead (e.g. heat, high pH, low ionic strength buffer etc.) and taken forward to a PCR reaction. In some cases, the bead-immobilized fraction may be discarded. In some cases, these daughter strands may be exact complements of a labeled strands immobilized to a bead. However, they may not contain any epigenetic modifications and hence may be free from “5-hmC-density” amplification bias. Amplification of these eluted daughter strands may give a superior result over existing methodologies for two reasons: 1) an improved resolution (higher signal-to-noise) and 2) an improved representation (decreased selection bias).

The methods and systems as described herein may provide a result that may be far more representative of an extent to which a nucleic acid may be marked epigenetically. In some cases, the methods and systems may be superior to other methods of identification of epigenetic modifications. Other methods of identification may include the HMCP method or a method that comprises associating a sugar, a protein, an antibody, or a fragment of any of these with an epigenetic modification and detecting a presence of the sugar, the protein, the antibody, or fragment thereof. In some cases, nucleic acid sequences, such as fragments containing a high density of epigenetic modifications may not be detected using other methods of identification of epigenetic modifications. The unbiased approach of the present methods and systems provides for detection of high density epigenetic modifications of nucleic acid sequences, such as short fragments yielding an unbias detection.

In FIG. 4, a daughter strand PCR amplification may occur. In some cases, PCR may be employed using only an eluted daughter strand as amplification template using standard protocols and procedures. In some cases, minimizing a number of PCR cycles may minimize duplicates. In some cases, using UMI-codes within an adapter sequence may help quantitation during downstream analysis. In some cases, a genome wide library of enriched fragments may be ready for sequencing.

### Method—Variation 4

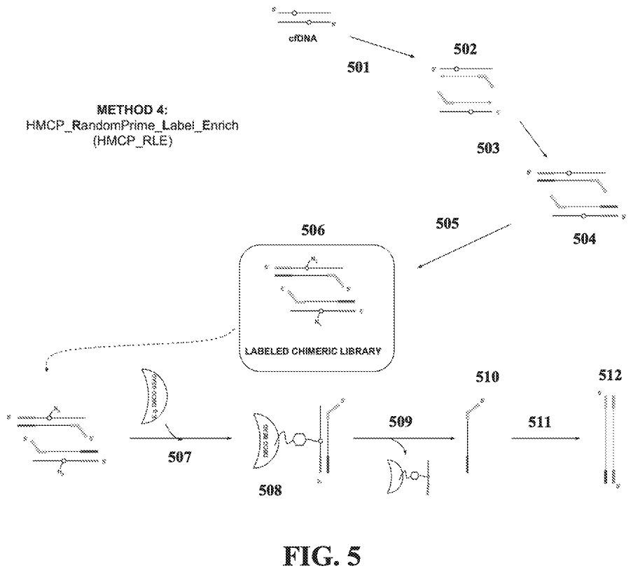

FIG. 5 shows one example of the 5-hmC Pulldown Random prime Label Enrich (HMCP_RLE) method detailed herein. In some cases, the HMCP_RLE method may provide: (a) an improved resolution as compared to other methods, such as a HMCP method or a method that may associate a sugar, an antibody, a protein, a fragment of any of these, a label, or any combination thereof with an epigenetically modified base of the nucleic acid; (b) a decrease in 5-hmC-density bias as compared to other methods, such as a HMCP method or a method that may associate a sugar, an antibody, a protein, a fragment of any of these, a label, or any combination thereof with an epigenetically modified base of the nucleic acid; (c) a substantially improved robustness at low input mass as compared to other methods, such as a HMCP method or a method that may associate a sugar, an antibody, a protein, a fragment of any of these, a label, or any combination thereof with an epigenetically modified base of the nucleic acid; or (d) any combination thereof.

FIG. 5 is similar to the method of FIG. 4 except that in some cases, priming (such as random priming) and ligation (such as adapter ligation) may occur before labeling as shown in FIG. 5 and in some cases, priming and ligation may occur after labeling as shown in FIG. 4.

As shown in FIG. 5, a first element 501 may (i) separate strands of a double stranded oligonucleotide fragment, such as a cell-free DNA fragment (having one or more epigenetic modifications at one or more bases on one or both strands) and (ii) initiate random priming to form a complementary strand, such as a substantially complementary strand, to at least one of the single stranded oligonucleotide fragment. Random priming may form a double stranded modified oligonucleotide fragment 502. The complementary strand formed by random priming may not have epigenetic modifications or may be substantially free of epigenetic modifications. A second element 503 may associate an adaptor to the double stranded modified oligonucleotide fragment (such as to one or both ends of one or both strands of the double stranded modified oligonucleotide fragment) to form a double stranded modified oligonucleotide fragment having one or more adaptors 504. A third element 505 may associate a label (such as an azido-glucose label) with the double stranded modified oligonucleotide fragment to yield a labeled fragment 506, such as a labeled chimeric library. The label may associate with an epigenetic modification or a type of epigenetic modification present at a base of the double stranded oligonucleotide fragment to form the labeled fragment 506. A fourth element 508 may be to associate a label 507 with the double stranded modified oligonucleotide fragment wherein the label 507 may also associate with a substrate. The label 507 may not bind directly to the complementary strand. The complementary strand may be indirectly associated with the substrate via the interaction between the substrate and the modified oligonucleotide fragment. The interaction between the complementary strand and the opposing strand may be disruptable, such as a disruptable bond. A fifth element 509 may be to enrich a sample for one or more complementary strands 510 by removing or separating or washing away from the substrate one or more complementary strands that lack a label associated with the substrate (such as by disrupting the interaction between the complementary strand and the opposing strand). Upon separation, the modified oligonucleotide fragment may remain associated with the substrate. A sixth element 511 may be to amplify the complementary strand in the absence of the modified oligonucleotide fragment to form one or more daughter strands 512 of the complementary strand.

### Method—Variation 5

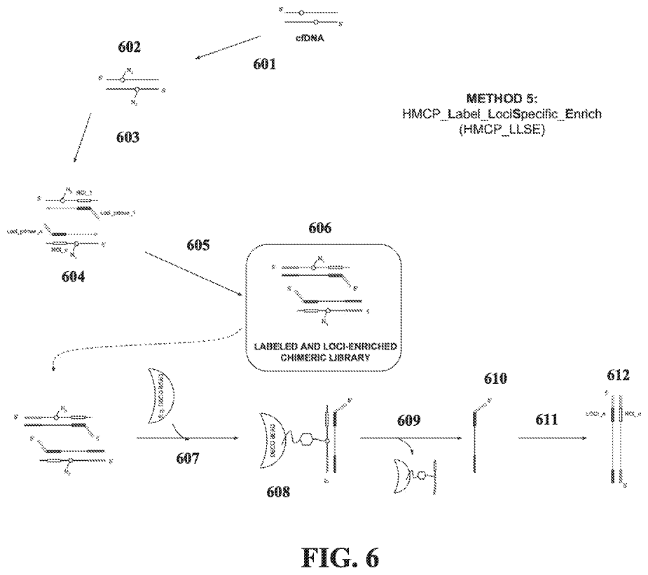

FIG. 6 shows one example of the 5-hmC Pulldown Label Loci Specific Enrich (HMCP_LLSE) method detailed herein. In some cases, the HMCP_LLSE method may provide (a) an improved resolution as compared to other methods, such as a HMCP method or a method that may associate a sugar, an antibody, a protein, a fragment of any of these, a label, or any combination thereof with an epigenetically modified base of the nucleic acid; (b) a decrease in a 5-hmC-density bias as compared to other methods, such as a HMCP method or a method that may associate a sugar, an antibody, a protein, a fragment of any of these, a label, or any combination thereof with an epigenetically modified base of the nucleic acid; (c) an substantially improved robustness at low input mass as compared to other methods, such as a HMCP method or a method that may associate a sugar, an antibody, a protein, a fragment of any of these, a label, or any combination thereof with an epigenetically modified base of the nucleic acid; (d) targeted regions of 5-hmC enriched DNA as compared with other methods, such as a HMCP method or a method that may associate a sugar, an antibody, a protein, a fragment of any of these, a label, or any combination thereof with an epigenetically modified base of the nucleic acid; or (e) any combination thereof.

As shown in FIG. 6, a first element 601 may associate a label (such as an azido-glucose label) with the double stranded oligonucleotide fragment, such as a cell-free DNA fragment to yield a labeled fragment 602. The label may associate with an epigenetic modification or a type of epigenetic modification present at one or more bases of the double stranded oligonucleotide fragment to form the labeled fragment 602. A second element 603 may (i) separate strands of a labeled fragment and (ii) initiate loci specific priming to form a complementary strand, such as a substantially complementary strand, to at least one of the single stranded oligonucleotide fragments. Loci specific priming may form a double stranded modified oligonucleotide fragment 604 having a label associated with an epigenetic modification of the parent strand. The complementary strand may be absent both epigenetic modifications and the associated label. A third element 605 may associate an adaptor to the double stranded modified oligonucleotide fragment (such as to one or both ends of one or both strands of the double stranded modified oligonucleotide fragment) to form a double stranded modified oligonucleotide fragment having one or more adaptors 606, such as a labeled and loci-enriched chimeric library. A fourth element 608 may be to associate a label 607 with the double stranded modified oligonucleotide fragment wherein the label 607 may also associate with a substrate. The label 607 may not bind directly to the complementary strand. The complementary strand may be indirectly associated with the substrate via the interaction between the substrate and the modified oligonucleotide fragment. The interaction between the complementary strand and the opposing strand may be disruptable, such as a disruptable bond. A fifth element 609 may be to enrich a sample for one or more complementary strands 610 by removing or separating or washing away from the substrate one or more complementary strands that lack a label associated with the substrate (such as by disrupting the bond between the complementary strand and the opposing strand). Upon separation, the opposing strand may remain associated with the substrate. A sixth element 611 may be to amplify the complementary strand in the absence of the modified oligonucleotide fragment to form one or more daughter strands 612 the complementary strand.

In this example, both strands of double stranded DNA (dsDNA) fragments containing 5-hmC may be labeled using beta-glucosyltransferase (βGT) and UDP-6-azide-glucose (UDP-N3-glc). This step may be dsDNA selective (βGT may not work on single stranded DNA (ssDNA)). Position of label may be determined by the presence/absence of 5-hmC in the dsDNA parent fragment. A label may be azido-glucose, transferred to the 5-hmC from UDP-N3-glc by βGT. The labeling may be performed directly on the purified circulating tumor DNA (ctDNA) extract. Advantage of this may be that the ctDNA may not have been through a series of library prep steps ahead of labeling. So there may be likely more material at the labeling (improved efficiency) and may present a more representative sample to a labeling than may be the case post NGS prep.

In some cases, hybridizing may comprise (i) priming (such as loci specific priming), (ii) ligation (such as adapter ligation), or (iii) a combination thereof. For example, in FIG. 6, loci specific priming may be performed by incubating azido-labeled dsDNA duplexes in the presence of an oligomer pool (where each oligo in the pool may comprise a loci specific “head” attached to a “NGS-adapter” tail), a DNA polymerase (e.g. Klenow) and a native dNTP mix in a given buffer, and performing a single extension reaction at 37° C. for a defined time (e.g. 10 mins). A loci specific head may be designed to be complementary to specific, defined regions of interest (ROI). Extension from an annealed loci specific primer may result in an A-overhang at an end of a daughter copy. A random priming may achieve two elements in one: 1) it may introduce an NGS-specific adapter sequence in a loci-specific manner and 2) it may generate a modification-free copy (daughter strand) of the modified parent strand.

In FIG. 6, a labelled loci-monoadapted chimeric duplex template may be incubated with a NGS-platform specific adapter (illustration shows forked adapter, but linear duplex adapter of hairpin adapter may be substituted) with 3′ T overhang and 5′ PO4 end, a dsDNA ligase (e.g. T4 ligase) and necessary cofactors (e.g. Mg2+, adenosine triphosphate (ATP), polyethylene glycol (PEG)) in a given buffer, at 20° C. for a defined period of time (e.g. 15 minutes). The A overhang of the monoadapted chimeric labelled duplex may match with the T overhang of the adapter and promotes ligation efficiency. In some cases, only one end of each duplex (that being formed by the 3′ end of the daughter strand) may be adapted. A successful ligation product may have a singly adapted azido-labeled parent strand (5′ adapted) and a doubly adapted non-modified daughter strand (both 3′ and 5′ ends). Where one to amplify this “library” it may be that only a bottom strand may be amplifiable with adapter-specific PCR primers.

In FIG. 6, following adapter ligation, an enrichment of the daughter strand by a substrate may be employed followed by PCR amplification of the daughter strand that may be substantially free of epigenetic modifications.

### Method—Variation 6

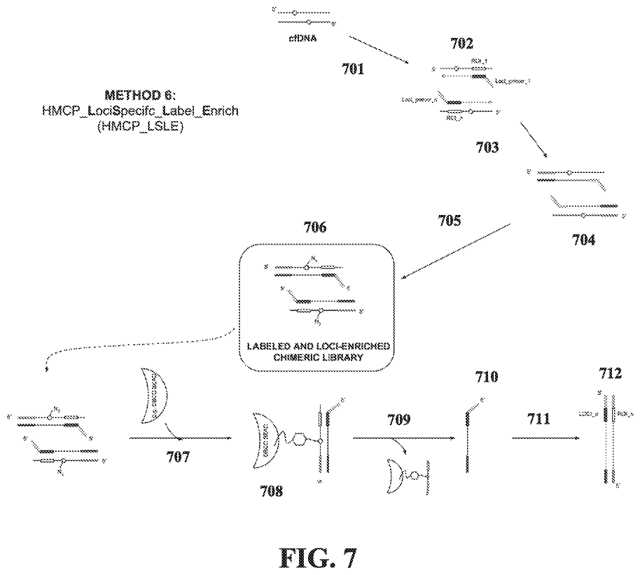

FIG. 7 shows one example of the 5-hmC Pulldown Loci Specific Label Enrich (HMCP_LSLE) method detailed herein. In some cases, the HMCP_LSLE method may provide (a) an improved resolution as compared to other methods, such as a HMCP method or a method that may associate a sugar, an antibody, a protein, a fragment of any of these, a label, or any combination thereof with an epigenetically modified base of the nucleic acid; (b) a decrease in a 5-hmC-density bias as compared to other methods, such as a HMCP method or a method that may associate a sugar, an antibody, a protein, a fragment of any of these, a label, or any combination thereof with an epigenetically modified base of the nucleic acid; (c) an substantially improved robustness at low input mass as compared to other methods, such as a HMCP method or a method that may associate a sugar, an antibody, a protein, a fragment of any of these, a label, or any combination thereof with an epigenetically modified base of the nucleic acid; (d) targeted regions of 5-hmC enriched DNA as compared with other methods, such as a HMCP method or a method that may associate a sugar, an antibody, a protein, a fragment of any of these, a label, or any combination thereof with an epigenetically modified base of the nucleic acid; or (e) any combination thereof.

FIG. 7 is similar to the method of FIG. 6 except that in some cases, priming (such as loci specific priming) and ligation (such as adapter ligation) may occur before labeling as shown in FIG. 7 and in some cases, priming and ligation may occur after labeling as shown in FIG. 6.

As shown in FIG. 7, a first element 701 may (i) separate strands of a double stranded oligonucleotide fragment, such as a cell-free DNA fragment and (ii) initiate loci specific priming to form a complementary strand, such as a substantially complementary strand, to at least one of the single stranded parent strands. Loci specific priming may form a double stranded modified oligonucleotide fragment 702. The double stranded oligonucleotide fragment may have one or more epigenetic modifications at one or more bases on one or both strands. The complementary strand, such as a substantially complementary strand, formed by loci specific priming may not have epigenetic modifications. A second element 703 may associate an adaptor to the double stranded modified oligonucleotide fragment (such as to one or both ends of one or both strands of the double stranded modified oligonucleotide fragment) to form a double stranded modified oligonucleotide fragment having one or more adaptors 704. A third element 705 may associate a label (such as an azido-glucose label) with the double stranded modified oligonucleotide fragment to yield a labeled fragment 706, such as a labeled chimeric library. The label may associate with an epigenetic modification or a type of epigenetic modification present at a base of the double stranded modified oligonucleotide fragment to form the labeled fragment 706. A fourth element 708 may be to associate a label 707 with the double stranded modified oligonucleotide fragment wherein the label 707 may also associate with a substrate. The label 707 may not bind directly to the complementary strand. The complementary strand may be indirectly associated with the substrate via the interaction between the substrate and the modified oligonucleotide fragment. The association between the complementary strand and the opposing strand may be disruptable, such as a disruptable bond. A fifth element 709 may be to enrich a sample for one or more complementary strands 710 by removing or separating or washing away from the substrate one or more complementary strands that lack a label associated with the substrate (such as by disrupting the bond between the complementary strand and the opposing strand). Upon separation, the opposing strand may remain associated with the substrate. A sixth element 711 may be to amplify the complementary strand in the absence of the parent strand to form one or more daughter strands 712 of the complementary strand.

The HMCP method may be referred to herein as the ‘standard’ method. The HMCP method may be referred to herein as HMCP, HMCP-v1, HMCPv1, HMCP, v1HMCP, v1 HMCP, or V1. The CLE method may be referred to herein as HMCP_CLE, HMCP-v2, HMCPv2, CLE-HMCP, v2HMCP, v2 HMCP, or V2.

For any of the methods described herein, including CLE, HMCP_LCE, HMCP_CLE, HMCP_LRE, HMCP_RLE, HMCP_LLSE, HMCP_LSLE, one or more individual elements of a given method may be performed in the order as described herein. In some cases, one or more individual elements of a given method need not be performed in a particular order described herein. In some cases, one or more individual elements of a given method may be performed in a different order than described herein.

In some cases, the complementary strand may be a substantially complementary strand or may comprise a portion that may be substantially complementary to a portion of a nucleic acid sequence.

Hybridizing may comprise hybridizing at least two complementary strands to at least two portions of a nucleic acid sequence. Hybridizing may comprise hybridizing at least a portion of a complementary strand to an adapter sequence of the nucleic acid sequence. Hybridizing may comprise extension, such as cDNA extension. Hybridizing may comprise priming, such as loci specific priming or random priming. Hybridizing may comprise ligation, such as adapter ligation. Hybridizing may comprise hybridizing a primer to a nucleic acid sequence and elongating from the primer to form a complementary strand. Hybridizing may comprise obtaining a complementary strand and hybridizing the complementary strand to the nucleic acid sequence.

A label may be associated with an epigenetically modified base of a nucleic acid sequence. A label may be associated with an epigenetically modified base before hybridizing. A label may be associated with an epigenetically modified base after hybridizing.

The method may comprise amplifying the complementary strand in a reaction in which the nucleic acid sequence may be substantially not present. The amplifying may comprise associating the nucleic acid sequence and complementary strand with a substrate, such as by a label. The amplifying may comprise washing a substrate that may be associated with the nucleic acid sequence and complementary strand, such as stringent washing. The amplifying may comprise eluting a complementary strand from the substrate on which the nucleic acid sequence remains. The amplifying may comprise amplifying the complementary strand.

An epigenetic modification may comprise a DNA methylation. A DNA methylation may comprise a hyper-methylation or a hypo-methylation. A DNA methylation may comprise a modification of a DNA base, such as a 5-methylcytosine (5-mC), a 4-methylcytosine, a 6-methyladenine, or a combination thereof.

### Definitions

As used herein, the singular forms “a”, “an”, and “the” include plural references unless the context clearly dictates otherwise. Any reference to “or” herein may be intended to encompass “and/or” unless otherwise stated.

As used herein, the term “about” may mean the referenced numeric indication plus or minus 15% of that referenced numeric indication.

The term “fragment,” as used herein, may be a portion of a sequence, a subset that may be shorter than a full length sequence. A fragment may be a portion of a gene. A fragment may be a portion of a peptide or protein. A fragment may be a portion of an amino acid sequence. A fragment may be a portion of an oligonucleotide sequence. A fragment may be less than about: 20, 30, 40, 50 amino acids in length. A fragment may be less than about: 20, 30, 40, 50 oligonucleotides in length.

The term “homology,” as used herein, may be to calculations of “homology” or “percent homology” between two or more nucleotide or amino acid sequences that can be determined by aligning the sequences for optimal comparison purposes (e.g., gaps can be introduced in the sequence of a first sequence). The nucleotides at corresponding positions may then be compared, and the percent identity between the two sequences may be a function of the number of identical positions shared by the sequences (i.e., % homology=(# of identical positions/total # of positions)×100). For example, a position in the first sequence may be occupied by the same nucleotide as the corresponding position in the second sequence, then the molecules are identical at that position. The percent homology between the two sequences may be a function of the number of identical positions shared by the sequences, taking into account the number of gaps, and the length of each gap, which need to be introduced for optimal alignment of the two sequences. In some embodiments, the length of a sequence aligned for comparison purposes may be at least about: 30%, 40%, 50%, 60%, 65%, 70%, 75%, 80%, 85%, 90%, 91%, 92%, 93%, 94%, 95%, 96%, 97%, 98%, or 95%, of the length of the reference sequence. A BLAST® search may determine homology between two sequences. The two sequences can be genes, nucleotides sequences, protein sequences, peptide sequences, amino acid sequences, or fragments thereof. The actual comparison of the two sequences can be accomplished by well-known methods, for example, using a mathematical algorithm. A non-limiting example of such a mathematical algorithm may be described in Karlin, S. and Altschul, S., Proc. Natl. Acad. Sci. USA, 90- 5873-5877 (1993). Such an algorithm may be incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g. , W=5 or W=20). Other examples include the algorithm of Myers and Miller, CABIOS (1989), ADVANCE, ADAM, BLAT, and FASTA. In another embodiment, the percent identity between two amino acid sequences can be accomplished using, for example, the GAP program in the GCG software package (Accelrys, Cambridge, UK).

The term “epigenetic modification” as used herein, may be any covalent modification of a nucleic acid base. In some cases, a covalent modification may comprise (i) adding a methyl group, a hydroxymethyl group, a carbon atom, an oxygen atom, or any combination thereof to one or more bases of a nucleic acid sequence, (ii) changing an oxidation state of a molecule associated with a nucleic acid sequence, such as an oxygen atom, or (iii) a combination thereof. A covalent modification may occur at any base, such as a cytosine, a thymine, a uracil, an adenine, a guanine, or any combination thereof. In some cases, an epigenetic modification may comprise an oxidation or a reduction. A nucleic acid sequence may comprise one or more epigenetically modified bases. An epigenetically modified base may comprise any base, such as a cytosine, a uracil, a thymine, adenine, or a guanine. An epigenetically modified base may comprise a methylated base, a hydroxymethylated base, a formylated base, or a carboxylic acid containing base or a salt thereof. An epigenetically modified base may comprise a 5-methylated base, such as a 5-methylated cytosine (5-mC). An epigenetically modified base may comprise a 5-hydroxymethylated base, such as a 5-hydroxymethylated cytosine (5-hmC). An epigenetically modified base may comprise a 5-formylated base, such as a 5-formylated cytosine (5-fC). An epigenetically modified base may comprise a 5-carboxylated base or a salt thereof, such as a 5-carboxylated cytosine (5-caC). In some cases, an epigenetically modified base may comprise a methyltransferase-directed transfer of an antivated group (mTAG).

An epigenetically modified base may comprise one or more bases or a purine (such as Structure 1) or one or more bases of a pyrimidine (such as Structure 2). An epigenetic modification may occur one or more of any positions. For example, an epigenetic modification may occur at one or more positions of a purine, including positions 1, 2, 3, 4, 5, 6, 7, 8, 9, as shown in Structure 1. In some cases, an epigenetic modification may occur at one or more positions of a pyrimidine, including positions 1, 2, 3, 4, 5, 6, as shown in Structure 2.

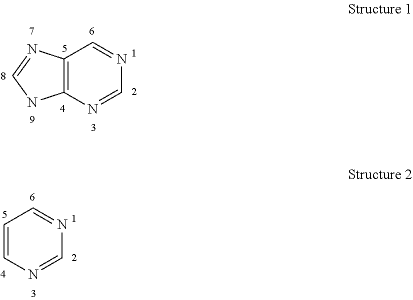 [SMILES: c1ncc2nc[nH]c2n1 ; c1cncnc1]

A nucleic acid sequence may comprise an epigenetically modified base. A nucleic acid sequence may comprise a plurality of epigenetically modified bases. A nucleic acid sequence may comprise an epigenetically modified base positioned within a CG site, a CpG island, or a combination thereof. A nucleic acid sequence may comprise different epigenetically modified bases, such as a methylated base, a hydroxymethylated base, a formylated base, a carboxylic acid containing base or a salt thereof, a plurality of any of these, or any combination thereof.

The term “sugar” as used herein, may be a sugar. A sugar may comprise a glucose, a fructose, a galactose, or a combination thereof. A sugar may comprise a disaccharide such as a sucrose, a maltose, a lactose, or any combination thereof. A sugar may comprise a monosaccharide, an oligosaccharide, or a polysaccharide. A sugar may comprise a modified sugar. A sugar may be modified such that the modified sugar may be configured to associate with an epigenetically modified base, such as a 5-methylated cytosine or a 5-hydroxymethylated cytosine. A sugar may comprise a modified glucose. A sugar may comprise a glucose, a glucose derivative, a gentibiose molecule, or any combination thereof. A sugar may comprise a uridine diphosphate glucose. A sugar may be modified with a detectable moiety, such as a radioactive moiety, a fluorescent moiety, a phosphorescent moiety, a chemiluminescent moiety, or any combination thereof. A sugar may be associated with a group for click chemistry. A sugar may be associated with an azido group, such as an N3 group. A sugar may be associated with an epigenetically modified base by employing a click chemistry reaction.

The term “barcode” as used herein may relate to a natural or synthetic nucleic acid sequence comprised by a polynucleotide allowing for unambiguous identification of the polynucleotide and other sequences comprised by the polynucleotide having said barcode sequence. The number of different barcode sequences theoretically possible can be directly dependent on the length of the barcode sequence; e.g., if a DNA barcode with randomly assembled adenine, thymidine, guanosine and cytidine nucleotides can be used, the theoretical maximal number of barcode sequences possible can be 1,048,576 for a length of ten nucleotides, and can be 1,073,741,824 for a length of fifteen nucleotides. Unique sample identifiers or barcodes can be completely scrambled (e.g., randomers of A, C, G, and T for DNA or A, C, G, and U for RNA) or they can have some regions of shared sequence. For example, a shared region on each end may reduce sequence biases in ligation events. In some cases, a shared region can be about or at least about 1, 2, 3, 4, 5, 6, 7, 8, 9, 10, 15, or 20 common base pairs. In some cases, a shared region can be up to about 1, 2, 3, 4, 5, 6, 7, 8, 9, 10, 15, or 20 common base pairs. Combinations of barcodes can be added to increase diversity.

A barcode may uniquely identify a subject, a sample (such as a cell-free sample), a nucleic acid sequence (such as a sequence having one or more epigenetically modified bases), or any combination thereof. A barcode may be associated with a nucleic acid sequence or a complementary strand. A nucleic acid sequence may comprise a single barcode. A nucleic acid sequence may comprise one or more barcodes, such as a first barcode and a second barcode. In some cases, the first barcode is different from the second barcode. In some cases, each barcode of a plurality of barcodes may be a unique barcode. In some cases, a barcode may comprise a sample identification barcode. For example, a first barcode may comprise a unique barcode and a second barcode may comprise a sample identification barcode.

The term “adapter” as used herein may be a nucleic acid with known or unknown sequence. An adapter may be attached to the 3′ end, 5′ end, or both ends of a nucleic acid (e.g. target nucleic acid). An adapter may comprise known sequences and/or unknown sequences. An adapter may be double-stranded or single-stranded. In some cases, an adapter can comprise a barcode (e.g. unique identifier sequence). In some cases, an adapter can be an amplification adapter. An amplification adapter may attach to a target nucleic acid and help the amplification of the target nucleic acid. For example, an amplification adapter may comprise one or more of: a primer binding site, a unique identifier sequence, a non-unique identifier sequence, and a sequence for immobilizing the target nucleic acid on a substrate. A target nucleic acid attached with an amplification adapter may be immobilized on a substrate. An amplification primer may hybridize to the adapter and be extended using the target nucleic acid as a template in an amplification reaction. In some cases, the unique identifiers in an adapter can be used to label the amplicons. In some cases, an adapter can be a sequencing adapter. A sequencing adapter may attach to a target nucleic acid and help the sequencing of the target nucleic acid. For example, a sequencing adapter may comprise one or more of: a sequencing primer binding site, a unique identifier sequence, a non-unique identifier sequence, and a sequence for immobilizing target nucleic acid on a substrate. A target nucleic acid attached with a sequencing adapter may be immobilized on a substrate on a sequencer. A sequencing primer may hybridize to the adapter and be extended using the target nucleic acid as a template in a sequencing reaction. In some cases, the unique identifiers in an adapter can be used to label the sequence reads of different target sequences, thus allowing high-throughput sequencing of a plurality of target nucleic acids. In some examples, an adapter sequence (such as a double-stranded or single-stranded oligonucleotide) may be ligated to one or both ends of a nucleic acids sequence. A nucleic acid sequence may comprise one or more epigenetically modified bases. A nucleic acid sequence may be from a sample, such as a cell free DNA sample. A nucleic acid sequence may be from a sample obtained from a subject. A nucleic acid sequence may comprise a double-stranded portion, a single-stranded portion, or a combination thereof. In some cases, an adapter may recognize or may be complementary to a primer, such as a universal primer. In some cases, an adapter may be specific to a sequencing method. In some cases, an adapter may be associated with a nucleic acid sequence or a complementary strand.

The term “nucleic acid sequence” as used herein may comprise DNA or RNA. In some cases, a nucleic acid sequence may comprise a plurality of nucleotides. In some cases, a nucleic acid sequence may comprise an artificial nucleic acid analogue. In some cases, a nucleic acid sequence comprising DNA, may comprise cell-free DNA, cDNA, fetal DNA, or maternal DNA. In some cases, a nucleic acid sequence may comprise miRNA, shRNA, or siRNA.

The term “substantially complementary strand” as used herein, may comprise from about 70%-100% bases that base pair with bases of a nucleic acid sequence. This percentage of base pairing may be measured by UV absorption of the nucleic acid sequence. In some cases, a substantially complementary strand may be hybridized to at least a portion of a nucleic acid sequence under stringent hybridization conditions.

The term “substantially free of an epigenetically modified base” as used herein, may comprise a complementary strand having no epigenetically modified base, or a complementary strand having from about 0.000001% to about 5% of a plurality of epigenetically modified bases of a nucleic acid sequence.

In some cases, a substantially complementary strand may be substantially free of a covalent modification. In some cases, a substantially complementary strand may be substantially free of (i) a methyl group, a hydroxymethyl group, a carbon atom, an oxygen atom, or any combination thereof, (ii) a change an oxidation state of a molecule associated with the substantially complementary strand or (iii) a combination thereof.

In some cases, a substantially complementary strand may be substantially free of an epigenetically modified base. In some cases, a substantially complementary strand may be free of an epigenetically modified base. In some cases, a substantially complementary strand may be amplified. An amplified product of the substantially complementary strand may comprise a plurality of epigenetically modified bases. In some cases, less than about: 90%, 80%, 70%, 60%, 50%, 40%, 30%, 20%, 10%, 5%, 4%, 3%, 2%, 1%, 0.01%, 0.001%, 0.0001%, 0.00001%, or 0.000001% of the amplified product comprises an epigenetically modified base. In some cases, a percentage may be by weight. In some cases, a percentage may be by a number of bases. In some cases, less than about 5% of an amplified product of a substantially complementary strand comprises an epigenetically modified base. In some cases, less than about 4% of an amplified product of a substantially complementary strand comprises an epigenetically modified base. In some cases, less than about 3% of an amplified product of a substantially complementary strand comprises an epigenetically modified base. In some cases, less than about 2% of an amplified product of a substantially complementary strand comprises an epigenetically modified base. In some cases, less than about 1% of an amplified product of a substantially complementary strand comprises an epigenetically modified base.

In some cases, from about 0.000001% to about 10% of an amplified product of a substantially complementary strand comprises an epigenetically modified base. In some cases, from about 0.000001% to about 5% of an amplified product of a substantially complementary strand comprises an epigenetically modified base. In some cases, from about 0.000001% to about 4% of an amplified product of a substantially complementary strand comprises an epigenetically modified base. In some cases, from about 0.000001% to about 1% of an amplified product of a substantially complementary strand comprises an epigenetically modified base. In some cases, from about 0.000001% to about 0.01% of an amplified product of a substantially complementary strand comprises an epigenetically modified base. In some cases, from about 0.000001% to about 0.001% of an amplified product of a substantially complementary strand comprises an epigenetically modified base. In some cases, from about 0.000001% to about 0.0001% of an amplified product of a substantially complementary strand comprises an epigenetically modified base. In some cases, from about 1% to about 10% of an amplified product of a substantially complementary strand comprises an epigenetically modified base.

A nucleic acid sequence may comprise a plurality of epigenetically modified bases. In some cases, a strand that may be substantially complementary to at least a portion of the nucleic acid may comprise less than about: 90%, 80%, 70%, 60%, 50%, 40%, 30%, 20%, 10%, 5%, 4%, 3%, 2%, 1%, 0.01%, 0.001%, 0.0001%, 0.00001%, or 0.000001% of the plurality of epigenetically modified bases of the nucleic acid sequence. In some cases, a percentage may be by weight. In some cases, a percentage may be by a number of bases. In some cases, less than about 5% of the plurality of epigenetically modified bases of a nucleic acid sequence may be present in a substantially complementary strand. In some cases, less than about 4% of the plurality of epigenetically modified bases of a nucleic acid sequence may be present in a substantially complementary strand. In some cases, less than about 3% of the plurality of epigenetically modified bases of a nucleic acid sequence may be present in a substantially complementary strand. In some cases, less than about 2% of the plurality of epigenetically modified bases of a nucleic acid sequence may be present in a substantially complementary strand. In some cases, less than about 1% of the plurality of epigenetically modified bases of a nucleic acid sequence may be present in a substantially complementary strand.

In some cases, about 0% of the plurality of epigenetically modified bases of a nucleic acid sequence may be present in a substantially complementary strand. In some cases, from about 0.000001% to about 10% of the plurality of epigenetically modified bases of a nucleic acid sequence may be present in a substantially complementary strand. In some cases, from about 0.000001% to about 5% of the plurality of epigenetically modified bases of a nucleic acid sequence may be present in a substantially complementary strand. In some cases, from about 0.000001% to about 4% of the plurality of epigenetically modified bases of a nucleic acid sequence may be present in a substantially complementary strand. In some cases, from about 0.000001% to about 1% of the plurality of epigenetically modified bases of a nucleic acid sequence may be present in a substantially complementary strand. In some cases, from about 0.000001% to about 0.1% of the plurality of epigenetically modified bases of a nucleic acid sequence may be present in a substantially complementary strand. In some cases, from about 0.000001% to about 0.01% of the plurality of epigenetically modified bases of a nucleic acid sequence may be present in a substantially complementary strand. In some cases, from about 0.000001% to about 0.001% of the plurality of epigenetically modified bases of a nucleic acid sequence may be present in a substantially complementary strand. In some cases, from about 1% to about 10% of the plurality of epigenetically modified bases of a nucleic acid sequence may be present in a substantially complementary strand.

In some cases, a substantially complementary strand may comprise an epigenetically modified base that may be different from an epigenetically modified base of a nucleic acid sequence.

The term “label” as used herein, may be a component that may be (a) associated with a substrate, (b) associated with an epigenetically modified base, or (c) a combination thereof. A label may be associated with an epigenetically modified base by a single bond, a double bond, a triple bond, a metal-associated bond, or an ion pairing. A label may comprise a magnetic metal, such as iron, nickel, cobalt, aluminum, or any combination thereof. A label may be associated with an epigenetically modified base by the assistance of an enzyme. A label may be associated with a substrate via (a) a biotin-streptavidin association, (b) a magnetic association, (c) an antibody-antigen association, or (d) any combination thereof. A label may be selectively for a portion of a nucleic acid sequence. A label may selectively associate with a double-stranded portion of a nucleic acid sequence as compared to single-stranded portion. A label may selectively associate with portions of a nucleic acid sequence having an epigenetically modified base as compared to portions having a non-modified base. A label may selectively associate with a type of epigenetically modified base, such as selectively associating with a 5-hydroxymethylated cytosine (5-hmC) as compared to a 5-methylated cytosine (5-mC). A label may comprise a sugar, such as a glucose. A glucose may comprise a modified glucose. A label may comprise more than one sugar, such as two sugars or more. A label may comprise a modified sugar, such as a modified glucose. A label may comprise a uridine diphosphate glucose (UDPG). A label may comprise a detectable label such as a radioactive label, a fluorescent label, a chemiluminescent label, a phosphorescent label, an infrared label, a visible label, a chemically reactive label (such as an azide-based label), or any combination thereof. In some cases, a label may be a label which results from incorporating a chromophore via a reaction with a radioactive label. A label may comprise a protein, peptide, or polypeptide. In some cases, a label may comprise an antibody or portion thereof. A label may comprise a tag, such as a FLAG-tag. A label may comprise a biotin or an avidin, such as streptavidin. A label may comprise a nucleic acid sequence. A label may comprise a substrate. In some cases, a different label may be employed to uniquely label different epigenetic modifications. For example, a first label may bind a methylated base and a second label may bind a hydroxymethylated base.

In some cases, a tag may comprise a giutathione-S-transferase (GST), a maltose binding protein (MBP), a green fluorescent protein (GFP). an AviTag, a Calmodulin tag, a polyglutamate tag, a FLAG tag, an human influenza hemagglutinin (HA) tag, a polyhistidine (His) tag, a Myc-tag, an S-tag, an streptavidin-binding peptide (SBP) tag, a Softag 1, a Strep tag, a TC tag, a V5 tag, an Xpress tag, an Isopeptag, a SpyTag, a biotin carboxyl carrier protein (BCCP) tag, a chitin binding protein (CBP) tag, a HaloTag, a thioredoxin tag, a T7 tag, a protein kinase A (PKA) tag, a c-Myc tag, a Trx tag, a Hsv tag, a CBD tag, a Dsb tag, a pelB/ompT, a KSI, a VSV-G tag, a 3-Gal tag, or any combination thereof. A tag may be a fusion tag, a covalent peptide tag, a protein tag, a peptide tag, an affinity tag, an epitope tag, a solubilization tag, or any combination thereof. A tag may comprise a recombinant protein. A tag may associate with a protein or protein fragment. A FLAG-tag may comprise a sequence or a portion thereof comprising DYKDDDDK, where D may be aspartic acid, Y may be tyrosine, and K may be lysine.

A label may be associated reversibly with a substrate. A label may be associated irreversibly with a substrate. A label may be reversibly associated with an epigenetically modified base. A label may be irreversibly associated with an epigenetically modified base. A label may be associated by binding to a substrate, an epigenetically modified base, or a combination thereof. A label may be bound by a single bond, a double bond, or a triple bond to a substrate. A label may be bound by a single bond, a double bond, or a triple bond to an epigenetically modified base.

The term “click-chemistry” as used herein may comprise a reaction having at least one of the following: (a) high yielding, (b) wide in scope, (c) create only byproducts that may be removed in the absence of chromatography, (d) stereospecific, (e) simple to perform, (f) conducted in easily removable or benign solvents. In some cases, click-chemistry comprises tagging, such as tagging a nucleic acid sequence or a complementary strand. In some cases, click-chemistry may associate a nucleic acid sequence with a label. Click-chemistry may comprise a reaction having a [3+2] cycloaddition; a thiol-ene reaction; a Diels-Alder reaction, an inverse electron demand Diels-Alder reaction; a [4+1] cycloaddition; a nucleophilic substitution; a carbonyl-chemistry-like formation of urea; an addition to a carbon-carbon double bond; or any combination thereof. In some cases, a [3+2] cycloaddition may comprise a Huisgen 1,3-dipolar cycloaddition. In some cases, a [4+1] cycloaddition may comprise a cycloaddition between an isonitrile and a tetrazine. Click-chemistry may comprise a copper(I)-catalyzed azide-alkyne cycloaddition (CuAAC); a strain-promoted azide-alkyne cycloaddition (SPAAC); a strain-promoted alkyne-nitrone cycloaddition (SPANC); or any combination thereof.

The term “moiety” as used herein, may be a component that may aid in or catalyze a reaction. In some cases, a moiety may comprise an enzyme or a catalytically active fragment thereof.

In some cases, a moiety may comprise an antibody or fragment thereof. In some cases, a moiety may comprise a protein, a peptide, or polypeptide. In some cases, a moiety may comprise a cofactor such as a coenzyme. In some cases, a moiety may comprise an enzyme, a protein or portion thereof, an antibody or portion thereof, a cofactor or any combination thereof. In some cases, a moiety, such as an enzyme, may aid in an association of a label with an epigenetically modified base. A moiety, such as an enzyme, may selectively associate a label with an epigenetically modified base present on a double-stranded oligonucleotide fragment as compared with an epigenetically modified base present on a single-stranded oligonucleotide fragment. A moiety, such as an enzyme, may selectively associate a label with an epigenetically modified base present on a single-stranded oligonucleotide fragment as compared with an epigenetically modified base present on a double-stranded oligonucleotide fragment. An enzyme may comprise a transferase. An enzyme may comprise a glucosyltransferase. An enzyme may comprise (a) an alpha-glucosyltransferase, (b) a beta-glucosyltransferase, (c) a beta-glucosyl-alpha-glucosyl-transferase, (d) J-glucosyltransferase, or (e) any combination thereof. A moiety, such as an enzyme, may comprise a modified moiety such as a genetically mutated moiety. A modified moiety may be modified to enhance an association of a label with an epigenetically modified base. A modified moiety may be modified to selectively aid in a) an association of a specific label with an epigenetically modified base, b) an association of a label with a specific epigenetically modified base, or c) a combination thereof.

In some cases, a moiety may catalyze a transfer of a methyl group to one or more bases of a nucleic acid sequence, a complementary strand, or a combination thereof. In some cases, a moiety may comprise a methyltransferase. In some cases, an enzyme may comprise a DNA methyltransferase 1 (DNMT1), a DNA methyltransferase 3-like (DNMT3L), a DNMT3A, a DNMT3B, a tRNA aspartic acid methyltransferase (TRDMT1), a DNMT3, any catalytically active fragment thereof, or any combination thereof.

In some cases, a moiety may catalyze a change in an epigenetic modification, such as a conversion of a methylated base to a hydroxymethylated base. In some cases, an enzyme may comprise a dioxygenase. In some cases, an enzyme may comprise a ten-eleven translocation (TET) family enzyme. In some cases, an enzyme may comprise TET1, TET2, TET3, CXXC finger protein 4 (CXXC4), any catalytically active fragment thereof, or any combination thereof.

In some cases, a moiety may catalyze an oxidative reaction, such as an oxidative decarboxylation. In some cases, an enzyme may comprise an isocitrate dehydrogenase (IDH) family enzyme. In some cases, an enzyme may comprise isocitrate dehydrogenase [NAD] subunit alpha (IDH3A), isocitrate dehydrogenase [NAD] subunit beta (IDH3B), isocitrate dehydrogenase [NAD] subunit gamma (IDH3G), isocitrate dehydrogenase 1 (IDH1), isocitrate dehydrogenase 2 (IDH2), any catalytically active fragment thereof, or any combination thereof.

A base of a nucleic acid sequence or a complementary strand may be deaminated, spontaneously or by contacting a moiety to a portion of a nucleic acid sequence. For example, a base, may be deaminated. In some cases, a base, a methylated base, a hydroxymethylated base, a formylated base, a carboxylated base, or any combination thereof may be deaminated. In some cases, a methylated cytosine may be deaminated. Deamination may occur selectively to a single base or to any combination of bases. Deamination may occur spontaneously. Deamination may occur by contacting a moiety to a portion of a nucleic acid sequence. A moiety may include an enzyme such as a deaminase, such as an adenosine deaminase, a guanine deaminase, or a cytidine deaminase. A deaminase may comprise activation-induced cytidine deaminase (AID), a conserved cytidine deaminase (CDA), apolipoprotein B mRNA editing enzyme catalytic polypeptide I (APOBEC1), apolipoprotein B mRNA-editing enzyme catalytic polypetide-like 3H (APOBEC3A-H), apolipoprotein B mRNA editing enzyme catalytic polypeptide-like 3G (APOBEC3G), or others. Bisulfite sequencing may deaminate one or more bases of a nucleic acid sequence or a complementary strand.

The term “sequencing” as used herein, may comprise bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLiD sequencing, Ion Torrent semiconductor sequencing, DNA nanoball sequencing, Heliscope single molecule sequencing, single molecule real time (SMRT) sequencing, nanopore DNA sequencing, shot gun sequencing, RNA sequencing, Enigma sequencing, or any combination thereof.

In some cases, a method may comprise sequencing. The sequencing may include bisulfite sequencing or bisulfite-free sequencing. In some cases, a method may comprise oxidizing one or more bases of a nucleic acid sequence or complementary strand or combination thereof. In some cases, a method may comprise selectively enriching for a nucleic acid sequence that contains at least one epigenetic modification.

The term “substrate” as used herein, may be a surface with which an entity (such as a label, a functional group, an epigenetic modification, a label or functional moiety associated with an epigenetic modification, a label or functional moiety associated with a parent strand) can be associated. In some cases, an entity may be immobilized to the substrate (such as a support). In some cases, an entity may be reversibly or irreversibly bound to the substrate (such as a support). In some cases, an entity may comprise a label. In such cases, a label may also associate with a nucleic acid sequence. In some cases, an entity may comprise a label, a nucleic acid sequence, a sugar, an enzyme, or any combination thereof. A substrate may comprise a bead. A substrate may comprise a plurality of beads. A substrate may comprise an array of beads. A substrate may comprise an array, such as an array of wells or an array of beads. A substrate (such as a solid support) may comprise a column, such as a packed column, a size-exclusion column, a magnetic column, or any combination thereof. A substrate may comprise a membrane. A substrate may comprise a bead, a capillary, a plate, a membrane, a wafer, a well, a plurality of any of these, an array of any of these, or any combination thereof. A substrate (such as a support) may positively select a nucleic acid sequence of interest by associating the nucleic acid sequence of interest with the substrate. A substrate may negatively select for a nucleic acid sequence of interest by associating other nucleic acid sequences of a sample with the substrate.

A bead may comprise one or more beads. A bead may comprise an array of beads. A bead may be associated with a substrate. A bead may be associated with a label. A bead may associate a label with a substrate. A bead may be associated with a substrate, a label, a nucleic acid sequence or any combination thereof. A bead may comprise a polymer, a metal, or a combination thereof. A bead may comprise a hydrogel, a silica gel, a glass, a resin, a metal, a metal alloy, a plastic, a cellulose, an agarose, a magnetic material, or any combination thereof.

The present disclosure provides substrates and methods of making substrates. The nature and geometry of a support or substrate can depend upon a variety of factors, including the type of array (e.g., one-dimensional, two-dimensional or three-dimensional). Generally, a substrate can be composed of any material which will not melt or otherwise substantially degrade under the conditions used to hybridize and/or denature nucleic acids. A substrate can be composed of any material which will permit coupling of an entity (such as a label associated with an epigenetic modification on a parent oligonucleotide fragment) at one or more discrete regions and/or discrete locations within the discrete regions. A substrate can be composed of any material which permit washing or physical or chemical manipulation without dislodging an entity (such as a label associated with an epigenetic modification on a parent oligonucleotide fragment) from the substrate.

Substrates can be fabricated by the transfer of an entity onto the solid surface in an organized high-density format followed by coupling the entity thereto. The techniques for fabrication of a substrate of the invention include, but are not limited to, photolithography, ink jet and contact printing, liquid dispensing and piezoelectrics. The patterns and dimensions of arrays are to be determined by each specific application. The sizes of each entity spot may be easily controlled by the users. A method of making a solid substrate can comprise contacting or coupling an entity to a discrete location.

A substrate may take a variety of configurations ranging from simple to complex, depending on the intended use of the array. Thus, a substrate can have an overall slide or plate configuration, such as a rectangular or disc configuration. A standard microplate configuration can be used. In some embodiments, the surface may be smooth or substantially planar, or have irregularities, such as depressions or elevations. In some instances, a substrate may have a rectangular cross-sectional shape, having a length of from about: 10-200 millimeters (mm), 40-150 mm, or 75-125 mm; a width of from about: 10-200 mm, 20-120 mm, or 25-80 mm, and a thickness of from about: 0.01-5.0 mm, 0.1-2 mm, or 0.2 to 1 mm.

A support may be organic or inorganic; may be metal (e.g., copper or silver) or non-metal; may be a polymer or nonpolymer; may be conducting, semiconducting or nonconducting (insulating); may be reflecting or nonreflecting; may be porous or nonporous; etc. A substrate as described above can be formed of any suitable material, including metals, metal oxides, semiconductors, polymers (particularly organic polymers in any suitable form including woven, nonwoven, molded, extruded, cast, etc.), silicon, silicon oxide, and composites thereof.

A number of materials (e.g., polymers) suitable for use as substrates (e.g., solid substrates) in the instant invention have been described in the art. Suitable materials for use as substrates include, but are not limited to, polycarbonate, gold, silicon, silicon oxide, silicon oxynitride, indium, tantalum oxide, niobium oxide, titanium, titanium oxide, platinum, iridium, indium tin oxide, diamond or diamond-like film, acrylic, styrene-methyl methacrylate copolymers, ethylene/acrylic acid, acrylonitrile-butadiene-styrene (ABS), ABS/polycarbonate, ABS/polysulfone, ABS/polyvinyl chloride, ethylene propylene, ethylene vinyl acetate (EVA), nitrocellulose, nylons (including nylon 6, nylon 6/6, nylon 6/6-6, nylon 6/9, nylon 6/10, nylon 6/12, nylon 11 and nylon 12), polyacrylonitrile (PAN), polyacrylate, polycarbonate, polybutylene terephthalate (PBT), poly(ethylene) (PE) (including low density, linear low density, high density, cross-linked and ultra-high molecular weight grades), poly(propylene) (PP), cis and trans isomers of poly(butadiene) (PB), cis and trans isomers of poly(isoprene), polyethylene terephthalate) (PET), polypropylene homopolymer, polypropylene copolymers, polystyrene (PS) (including general purpose and high impact grades), polycarbonate (PC), poly(epsilon-caprolactone) (PECL or PCL), poly(methyl methacrylate) (PMMA) and its homologs, poly(methyl acrylate) and its homologs, poly(lactic acid) (PLA), poly(glycolic acid), polyorthoesters, poly(anhydrides), nylon, polyimides, polydimethylsiloxane (PDMS), polybutadiene (PB), polyvinylalcohol (PVA), polyacrylamide and its homologs such as poly(N-isopropyl acrylamide), fluorinated polyacrylate (PFOA), poly(ethylene-butylene) (PEB), poly(styrene-acrylonitrile) (SAN), polytetrafluoroethylene (PTFE) and its derivatives, polyolefin plastomers, fluorinated ethylene-propylene (FEP), ethylene-tetrafluoroethylene (ETFE), perfluoroalkoxyethylene (PFA), polyvinyl fluoride (PVF), polyvinylidene fluoride (PVDF), polychlorotrifluoroethylene (PCTFE), polyethylene-chlorotrifluoroethylene (ECTFE), styrene maleic anhydride (SMA), metal oxides, glass, silicon oxide or other inorganic or semiconductor material (e.g., silicon nitride), compound semiconductors (e.g., gallium arsenide, and indium gallium arsenide), and combinations thereof.

Examples of well-known substrates include polypropylene, polystyrene, polyethylene, dextran, nylon, amylases, glass, natural and modified celluloses (e.g., nitrocellulose), polyacrylamides, agaroses and magnetite. In some instances, the substrate can be silica or glass because of its great chemical resistance against solvents, its mechanical stability, its low intrinsic fluorescence properties, and its flexibility of being readily functionalized. In one embodiment, the substrate is glass, particularly glass coated with nitrocellulose, more particularly a nitrocellulose-coated slide (e.g., FAST slides).

A substrate may be modified with one or more different layers of compounds or coatings that serve to modify the properties of the surface in a desirable manner. For example, a substrate may further comprise a coating material on the whole or a portion of the surface of the substrate. In some embodiments, a coating material enhances the affinity of the entity (such as a functional group) for the substrate. For example, the coating material can be nitrocellulose, silane, thiol, disulfide, or a polymer. When the material is a thiol, the substrate may comprise a gold-coated surface and/or the thiol comprises hydrophobic and hydrophilic moieties. When the coating material is a silane, the substrate comprises glass and the silane may present terminal moieties including, for example, hydroxyl, carboxyl, phosphate, glycidoxy, sulfonate, isocyanato, thiol, or amino groups. In an alternative embodiment, the coating material may be a derivatized monolayer or multilayer having covalently bonded linker moieties. For example, the monolayer coating may have thiol (e.g., a thioalkyl selected from the group consisting of a thioalkyl acid (e.g., 16-mercaptohexadecanoic acid), thioalkyl alcohol, thioalkyl amine, and halogen containing thioalkyl compound), disulfide or silane groups that produce a chemical or physicochemical bonding to the substrate. The attachment of the monolayer to the substrate may also be achieved by non-covalent interactions or by covalent reactions.

After attachment to the substrate, a coating may comprise at least one functional group. Examples of functional groups on the monolayer coating include, but are not limited to, carboxyl, isocyanate, halogen, amine or hydroxyl groups. In one embodiment, these reactive functional groups on the coating may be activated by standard chemical techniques to corresponding activated functional groups on the monolayer coating (e.g., conversion of carboxyl groups to anhydrides or acid halides, etc.). Exemplary activated functional groups of the coating on the substrate for covalent coupling to terminal amino groups include anhydrides, N-hydroxysuccinimide esters or other common activated esters or acid halides, Exemplary activated functional groups of the coating on the substrate include anhydride derivatives for coupling with a terminal hydroxyl group; hydrazine derivatives for coupling onto oxidized sugar residues of the linker compound; or maleimide derivatives for covalent attachment to thiol groups of the linker compound. To produce a derivatized coating, at least one terminal carboxyl group on the coating can be activated to an anhydride group and then reacted, for example, with a linker compound. Alternatively, the functional groups on the coating may be reacted with a linker having activated functional groups (e.g., N-hydroxysuccinimide esters, acid halides, anhydrides, and isocyanates) for covalent coupling to reactive amino groups on the coating.

A substrate can contain a linker (e.g., to indirectly couple an entity to the substrate). In one embodiment, a linker has one terminal functional group, a spacer region and an entity adhering region. The terminal functional groups for reacting with functional groups on an activated coating include halogen, amino, hydroxyl, or thiol groups. In some instances, a terminal functional group is selected from the group consisting of a carboxylic acid, halogen, amine, thiol, alkene, acrylate, anhydride, ester, acid halide, isocyanate, hydrazine, maleimide and hydroxyl group. The spacer region may include, but is not limited to, polyethers, polypeptides, polyamides, polyamines, polyesters, polysaccharides, polyols, multiple charged species or any other combinations thereof. Exemplary spacer regions include polymers of ethylene glycols, peptides, glycerol, ethanolamine, serine, inositol, etc. The spacer region may be hydrophilic in nature. The spacer region may be hydrophobic in nature. In some instances, the spacer has n oxyethylene groups, where n is between 2 and 25. In some instances, a region of a linker that adheres to an entity may be hydrophobic or amphiphilic with straight or branched chain alkyl, alkynyl, alkenyl, aryl, arylalkyl, heteroalkyl, heteroalkynyl, heteroalkenyl, heteroaryl, or heteroarylalkyl. In some instances, a region of a linker that adheres to an entity may comprise a C₁₀-C₂₅ straight or branched chain alkyl or heteroalkyl hydrophobic tail. In some instances, a linker comprises a terminal functional group on one end, a spacer, an entity adhering region, and a hydrophilic group on another end. The hydrophilic group at one end of the linker may be a single group or a straight or branched chain of multiple hydrophilic groups (e.g., a single hydroxyl group or a chain of multiple ethylene glycol units).

In some embodiments, a support can be planar. In some instances, a support can be spherical. In some instances, a support can be a bead. In some instances, a support can be magnetic. In some instances, a magnetic substrate can comprises magnetite, maghemitite, FePt, SrFe, iron, cobalt, nickel, chromium dioxide, ferrites, or mixtures thereof. In some instances, a support can be nonmagnetic. In some embodiments, the nonmagnetic substrate can comprise a polymer, metal, glass, alloy, mineral, or mixture thereof. In some instances a nonmagnetic material can be a coating around a magnetic substrate. In some instances, a magnetic material may be distributed in the continuous phase of a magnetic material. In some embodiments, the substrate comprises magnetic and nonmagnetic materials. In some instances, a substrate can comprise a combination of a magnetic material and a nonmagnetic material. In some embodiments, the magnetic material is at least about: 5%, 10%, 20%, 30%, 40%, 50%, 60%, 70%, or about 80% by weight of the total composition of the substrate. In some embodiments, the bead size can be quite large, on the order of from about 100 microns to about 900 microns or in some cases even up to a diameter of about 3 mm. In other embodiments, the bead size can be on the order of from about 1 microns to about 150 microns. The average particle diameters of beads of the invention can be in the range of from about 2 μm to about several millimeters, e.g., diameters in ranges having lower limits of about: 2 μm, 4 μm, 6 μm, 8 μm, 10 μm, 20 μm, 30 μm, 40 μm, 50 μm, 60 μm, 70 μm, 80 μm, 90 μm, 100 μm, 150 μm, 200 μm, 300 μm, or 500 μm, and upper limits of 20 μm, 30 μm, 40 μm, 50 μm, 60 μm, 70 μm, 80 μm, 90 μm, 100 μm, 150 μm, 200 μm, 300 μm, 500 μm, 750 μm, 1 mm, 2 mm, or 3 mm.

The term “tissue” as used herein, may be any tissue sample. A tissue may be a tissue suspected or confirmed of having a disease or condition. A tissue may be a sample that may be substantially healthy, substantially benign, or otherwise substantially free of a disease or a condition. A tissue may be a tissue removed from a subject, such as a tissue biopsy, a tissue resection, an aspirate (such as a fine needle aspirate), a tissue washing, a cytology specimen, a bodily fluid, or any combination thereof. A tissue may comprise cancerous cells, tumor cells, non-cancerous cells, or a combination thereof. A tissue may comprise brain tissue, cerebral spinal tissue, cerebral spinal fluid, breast tissue, bladder tissue, kidney tissue, liver tissue, colon tissue, thyroid tissue, cervical tissue, prostate tissue, lung tissue, heart tissue, muscle tissue, pancreas tissue, anal tissue, bile duct tissue, a bone tissue, uterine tissue, ovarian tissue, endometrial tissue, vaginal tissue, vulvar tissue, stomach tissue, ocular tissue, nasal tissue, sinus tissue, penile tissue, salivary gland tissue, gut tissue, gallbladder tissue, gastrointestinal tissue, bladder tissue, brain tissue, spinal tissue, a blood sample, or any combination thereof. A tissue may be a sample that may be genetically modified.

The term “subject,” as used herein, may be any animal or living organism. Animals can be mammals, such as humans, non-human primates, rodents such as mice and rats, dogs, cats, pigs, sheep, rabbits, and others. Animals can be fish, reptiles, or others. Animals can be neonatal, infant, adolescent, or adult animals. Humans can be more than about: 1, 2, 5, 10, 20, 30, 40, 50, 60, 65, 70, 75, or about 80 years of age. The subject may have or be suspected of having a condition or a disease, such as cancer. The subject may be a patient, such as a patient being treated for a condition or a disease, such as a cancer patient. The subject may be predisposed to a risk of developing a condition or a disease such as cancer. The subject may be in remission from a condition or a disease, such as a cancer patient. The subject may be healthy.

The term “reads per kilobase per million mapped reads (RPKM),” as used herein, may be a method of quantifying gene expression from sequencing data (such as RNA sequencing data) by normalizing for a total read length and/or a number of sequencing reads. In some cases, RPKM may correct for differences in sample sequencing depth. In some cases, RPKM may correct for differences in gene length. In some cases, RPKM may correct for differences in both sample sequencing depth and gene length. RPKM may be a method of normalizing data for comparison of gene coverage values. In some cases, RPKM may be defined as numReads/((geneLength/1000)*(totalNumReads/1,000,000)), wherein numReads may be a number of reads mapped to a gene sequence, geneLenth may be a length of the gene sequence, and totalNumReads may be a total number of mapped reads of a sample.

A substantially complementary strand may be hybridized to a portion of a nucleic acid sequence. A substantially complementary strand may be substantially a same length as a nucleic acid sequence. A substantially complementary strand may comprise a length that may be at least about: 99%, 95%, 90%, 80%, 70%, 60%, 50%, 40%, 30%, 20%, 10%, 5%, 3%, 2%, 1% of a length of a nucleic acid sequence. A substantially complementary strand may be shorter, longer or the same in length compared to a nucleic acid sequence.

A substantially complementary strand may be hybridized to at least a portion of a nucleic acid sequence. In some cases, two substantially complementary strands may be hybridized to portions of a nucleic acid sequence. In some cases, the two substantially complementary strands may be ligated. In some cases, three or more substantially complementary strands may be hybridized to portions of a nucleic acid sequence. In some cases, the three or more substantially complementary strands may be ligated. In some cases, a substantially complementary strand may be hybridized to a portion of a nucleic acid sequence that may comprise an adaptor sequence. A substantially complementary strand may be elongated, such as elongated before amplifying.

A nucleic acid sequence may comprise a cytosine guanine (CG) site, a cytosine phosphate guanine (CpG) island, a portion of any of these, or a combination thereof. A CpG island may comprise one or more CG sites. A nucleic acid sequence may comprise one or more CG sites or portions thereof. A nucleic acid sequence may comprise dense CG sites, dense CpG islands or a combination thereof. A nucleic acid sequence may comprise a plurality of CG sites or portions thereof. A nucleic acid sequence may comprise one or more CpG islands or portions thereof. A nucleic acid sequence may comprise a plurality of CpG islands or portions thereof. One or more bases of a nucleic acid sequence comprising a CG site, a CpG island, a portion thereof, or any of these may comprise an epigenetically modified base, such as a methylated base or a hydroxymethylated base. One or more cytosines of a nucleic acid sequence comprising a CG site, a CpG island, a portion thereof, or any of these may comprise an epigenetically modified cytosine, such as a methylated cytosine or a hydroxymethylated cytosine. A CpG island (or a CG island) may be a region with a high frequency of CG sites. A CpG island may be a region of a nucleic acid sequence with at least about 200 basepairs (bp) and a GC percentage that may be greater than about 50% and with an observed-to-expected CpG ratio that may be greater than about 60%. An “observed-to-expected CpG ratio” may be derived where the observed may be calculated as:

(number of CpGs)

and the expected may be calculated as:

(number of C*number of G)/length of sequence

or the expected may be calculated as:

((number of C+number of G)/2)²/length of sequence

In some cases, a CpG island may be a region of a nucleic acid sequence with at least about: 20, 30, 40, 50, 60, 70, 80, 90, 100, 110, 120, 130, 140, 150, 160, 170, 180, 190, 200, 225, 250, 300, 350, 400, 450, 500, 550, 600 bp. In some cases, a CpG island may be a region of a nucleic acid sequence with from about 20 to about 600 bp. In some cases, a CpG island may be a region of a nucleic acid sequence with from about 20 to about 500 bp. In some cases, a CpG island may be a region of a nucleic acid sequence with from about 10 to about 500 bp. In some cases, a CpG island may be a region of a nucleic acid sequence with from about 10 to about 300 bp. In some cases, a CpG island may be a region of a nucleic acid sequence with from about 20 to about 200 bp.

In some cases, a GC percentage in a CpG island may be greater than about: 50%, 55%, 60%, 65%, 70%, 75%, 80%, 85%, 90%, 95% or greater. In some cases, a GC percentage in a CpG island may be from about 50% to about 95%. In some cases, a GC percentage in a CpG island may be from about 50% to about 99%. In some cases, a GC percentage in a CpG island may be from about 55% to about 85%. In some cases, a GC percentage in a CpG island may be from about 60% to about 99%. In some cases, a GC percentage in a CpG island may be from about 70% to about 99%.

The term “density of epigenetic modifications,” as used herein, may be a number or percentage of bases within a sequence that have an epigenetic modification. The epigenetic modification may be a methylated cytosine, a hydroxymethylated cytosine, a carboxylated cytosine, a formylated cytosine, or other epigenetic modification. In some cases, a sequence having 6 5-hmC may represent a sequence having a high density of epigenetic modifications. In some cases, a sequence having 2 5-hmC may represent a sequence having a low density of epigenetic modifications. A low density of epigenetic modifications may include a sequence having less than 5, 4, 3, or 2 CG sites per 10 base pairs (bp), 20 bp, 30 bp, 40 bp, 50 bp segment of the sequence. A high density of epigenetic modifications may include a sequence having more than 2, 3, 4, 5, 6, 7, 8, 9, 10 CG sites per 10 bp, 15 bp, 30 bp, 35 bp, 40 bp, 50 bp segment of the sequence. A high density of epigenetic modifications may include a sequence having a plurality of CpG islands or CG sites. A high density of epigenetic modifications may include a sequence having at least 5, 6, 7, 8, 9, 10, 11, 12, 13, 14, 15, 16, 17, 18, 19, 20 or more CpG islands or CG sites per sequence.

As used herein, the term “cell-free” refers to the condition of the nucleic acid sequence as it appeared in the body before the sample is obtained from the body. For example, circulating cell-free nucleic acid sequences in a sample may have originated as cell-free nucleic acid sequences circulating in the bloodstream of the human body. In contrast, nucleic acid sequences that are extracted from a solid tissue, such as a biopsy, are generally not considered to be “cell-free.” In some cases, cell-free DNA may comprise fetal DNA, maternal DNA, or a combination thereof. In some cases, cell-free DNA may comprise DNA fragments released into a blood plasma. In some cases, the cell-free DNA may comprise circulating tumor DNA. In some cases, cell-free DNA may comprise circulating DNA indicative of a tissue origin, a disease or a condition. A cell-free nucleic acid sequence may be isolated from a blood sample. A cell-free nucleic acid sequence may be isolated from a plasma sample. A cell-free nucleic acid sequence may comprise a complementary DNA (cDNA). In some cases, one or more cDNAs may form a cDNA library.

In some cases, a nucleic acid sequence may be double-stranded, such as a cDNA library comprising the nucleic acid sequence. In some cases, a nucleic acid sequence may be double-stranded such as when a substantially complementary strand may be hybridized to at least a portion of the nucleic acid sequence. In some cases, a portion of a nucleic acid sequence may be double-stranded, such as when a primer may be hybridized to a portion of the nucleic acid sequence.

A nucleic acid sequence may be from a sample. A sample may be isolated from a subject. A subject may be a human subject. A sample may comprise a buccal sample, a saliva sample, a blood sample, a plasma sample, a reproductive sample (such as an egg or a sperm), a mucus sample, a cerebral spinal fluid sample, a tissue sample, a tissue biopsy, a surgical resection, a fine needle aspirate sample, or any combination thereof. In some cases, a sample may comprise a blood sample. In some cases, a sample may comprise a buccal sample.

In some cases, a subject may have previously received a diagnosis of a disease or condition prior to performing a method as described herein. A subject may have previously received a positive diagnosis of a disease, such as a cancer. A subject may have previously received an indeterminate or inclusive diagnosis of a disease, such as a cancer. A subject may be a subject in need thereof, such as a need for a definitive diagnosis or a need for a selection of a therapeutic treatment regime.

In some cases, a subject may not have previously received a diagnosis of a disease or condition prior to performing a method as described herein. In some cases, a subject may be suspected of having a disease or condition, such as having one or more symptoms of a disease or condition. In some cases, a subject may be at risk of developing a disease or condition, such as a subject having a biomarker or genetic indication that may be indicative of a risk of developing a disease or condition. In some cases, a disease or a condition may comprise a cancer.

In some cases, a method as described herein may comprise obtaining a result. A method may comprise obtaining a result and reporting the result. A result may be reported to a user, a medical professional, a subject, or any combination thereof. A result may be reported via a communication medium. A communication medium may include a written report or a printed report. A communication medium may include a visual display such as a graphical user interface. A communication medium may comprise a result provided by a computer, a tablet device, a cellphone, or other electronic device. A result may comprise a diagnosis of a disease or condition or a confirmation of an absence of a disease or condition. A result may comprise a diagnosis of a subject as having a disease or condition. A result may comprise a confirmation of an absence of the disease or condition. A result may comprise a likelihood or a risk of a subject to develop a disease or a condition. In some cases, a disease or a condition may comprise a cancer. A result may comprise predicting mortality of a subject, determining a biological age of a subject, or a combination thereof. A mortality prediction or biological age determination may be based on a presence of an epigenetic modification, sequencing information or any combination thereof. A result, such as a prediction of a likelihood of a disease or condition or a diagnosis of a disease or condition may be based on a presence of an epigenetic modification, sequencing information or a combination thereof. A presence of an epigenetic modification may include a pattern of epigenetic modification, a presence of a specific epigenetic modification, a level of an epigenetic modification, or any combination thereof.

A method as described herein may comprise comparing a result to a reference. A reference may comprise a plurality of references. A reference may comprise a database comprising a plurality of results. A reference may comprise a control sample. A reference may comprise a positive control sample, a negative control sample, or a combination thereof. A reference, such as a reference sample, may be obtained from a subject or from a different source, such as a different subject. A diagnosis may comprise comparing a result to a reference. In some cases, a result comprising a diagnosis may at least partially confirm a previous diagnosis.

### Diagnostics

One or more results obtained from a method described herein may provide a quantitative value or values indicative of one or more of the following: a likelihood of diagnostic accuracy, a likelihood of a presence of a condition in a subject, a likelihood of a subject developing a condition, a likelihood of success of a particular treatment, or any combination thereof. A method as described herein may predict a risk or likelihood of developing a condition. A method as described herein may be an early diagnostic indicator of developing a condition. A method as described herein may confirm a diagnosis or a presence of a condition. A method as described herein may monitor the progression of a condition. A method as described herein may monitor the efficacy of a treatment for a condition in a subject.

Samples obtained for analysis using the methods described herein may be obtained from a subject. The subject may not have any symptoms of a condition. The subject may have one or more symptoms of a condition. The subject may be a risk, such as a genetic risk, of developing a condition. The subject may have previously received a positive diagnosis. The subject may have previously received an indeterminate result from a diagnostic test. The subject may be currently receiving in a treatment.

Methods for diagnosing and/or suggesting, selecting, designating, recommending or otherwise determining a course of treatment for a subject having or suspected of having a condition can be employed in combination with the methods as described herein. These techniques may include cytological analysis or histological classification, molecular profiling, a blood test, a genetic analysis, ultrasound analysis, MRI results, CT scan results, other imaging scans, measurements of hormone cytokine or blood cell levels, or any combination thereof. The methods described herein may include at least one other type of diagnostic method. The methods described herein may include at least two other diagnostic methods.

In some embodiments, the methods of the present invention provide for storing the sample for a time such as seconds, minutes, hours, days, weeks, months, years or longer after the sample is obtained and before the sample is analyzed by one or more methods of the invention. In some cases, the sample obtained from a subject is subdivided prior to the step of storage or further analysis such that different portions of the sample are subject to different downstream methods or processes including but not limited to any combination of methods described herein, storage, bisulfite treatment, amplification, sequencing, labeling, cytological analysis, adequacy tests, nucleic acid extraction, molecular profiling or a combination thereof.

In some cases, a portion of the sample may be stored while another portion of said sample is further manipulated. Such manipulations may include but are not limited to any method as described herein; bisulfite treatment; sequencing; amplification; labeling; selective enrichment; molecular profiling; cytological staining; nucleic acid (RNA or DNA) extraction, detection, or quantification; gene expression product (RNA or Protein) extraction, detection, or quantification; fixation; and examination. The sample may be fixed prior to or during storage by any method known to the art such as using glutaraldehyde, formaldehyde, or methanol. In other cases, the sample is obtained and stored and subdivided after the step of storage for further analysis such that different portions of the sample are subject to different downstream methods.

### Treatment

A method as described herein may comprise treating a subject. In some cases, a treatment may comprise surgery, chemotherapy, radiation therapy, immunotherapy, targeted therapy, hormone therapy, stem cell transplantation, precision medicine, or any combination thereof. In some cases, a treatment may comprise further monitoring of a condition of a subject. In some cases, a subject diagnosed with a disease or condition may receive a treatment to treat a disease or a condition. In some cases, a subject receiving a confirmation of a likelihood or a risk of developing a disease or a condition, may receive a treatment, such as a preventive treatment. A treatment for a subject may be selected based on a result of a method, such as a confirmed positive diagnosis of a disease or a condition. A result may comprise one or more treatments, such as a recommended treatment, for a subject based on a result. A treatment may comprise a single treatment. A treatment may comprise a recurring treatment. A treatment may comprise a recurring treatment over a remaining lifespan of a subject. A treatment may comprise a daily treatment. A treatment may comprise a biweekly treatment. A treatment may be selected base on a result.

In some embodiments, a treatment for a subject can be a surgery (such as a tissue resection), a nutrition regime, a physical activity, a radiation treatment, a chemotherapy, an immunotherapy, a pharmaceutical composition, a cell transplantation, a blood fusion, or any combination thereof.

The methods described herein, such as assaying and comparing, may be conducted prior to an operation on a diseased tissue of the subject, such as a tumor resection. The methods described herein may be conducted prior to the subject having a positive disease diagnosis, such as a cancer or a tumor diagnosis. The methods described herein may be conducted on a subject suspected of having a condition or a disease, such as a cancer or a tumor. The methods described herein may be conducted on a subject that has received a positive disease diagnosis, such as a positive cancer or a positive tumor diagnosis. The methods described herein may be conducted on a subject having received a prior treatment regime, wherein the prior treatment regime was ineffective in eliminating the disease or condition, such as a cancer or tumor. A tissue sample may be obtained from a subject prior to performing the methods described herein. A tissue sample may be obtained during a biopsy, fine needle aspiration, blood sample, surgery resection, or any combination thereof.

Assaying a tissue sample of a subject may be performed at one or more time points. A separate tissue sample may be obtained from the subject for assaying at each of the one or more time points. Assaying at one or more time points may be performed on the same tissue sample. Assaying at one or more time points may provide an assessment of an effectiveness of a drug, a longitudinal course of a disease treatment regime, or a combination thereof. At each of the one or more time points, a tissue sample may be compared to a same reference. A tissue sample may be compared to a different reference at each of the one or more time points. The one or more time points may be the same. The one or more time points may be different. The one or more time points may comprise at least one time point prior to a drug administration, at least one time point after a drug administration, at least one time point prior to a positive disease diagnosis, at least one time point after a disease remission diagnosis, at least one time point during a disease treatment regime, or a combination thereof.

The methods as described herein may be used for diagnosis of a particular condition and also to monitor efficacy of a particular treatment after an initial diagnosis or monitor progression of a particular condition. The methods as described herein may be used to monitoring a subject as risk of developing a particular condition, as a preventive measure. The methods as described herein may be used alone for diagnosis and/or monitoring efficacy of a particular treatment. The methods as described herein may be used in combination with other assays for diagnosis or monitoring (such as a cytological analysis or molecular profiling).

A subject may be monitored using methods as disclosed herein. For example, a subject may be diagnosed with condition, such as a cancer or a genetic disorder. This initial diagnosis may or may not involve the use of the methods described herein. The subject may be prescribed a treatment such as surgical resection of a tumor or chemotherapy. The results of the treatment may be monitored on an ongoing basis by the methods described herein to detect the efficacy of the treatment. In another example, a subject may be diagnosed with a benign tumor or a precancerous lesion or nodule, and the tumor, nodule, or lesion may be monitored on an ongoing basis by the methods described herein to detect any changes in the state of the tumor or lesion.

The methods described herein may also be used to ascertain the potential efficacy of a specific treatment prior to administering to a subject. For example, a subject may be diagnosed with cancer. The methods described herein may indicate a presence of one or more epigenetic residues on a particular nucleic acid sequence known to be involved in cancer malignancy. A further sample may be obtained from the subject and cultured in vitro using methods known to the art. The application of various inhibitors or drugs may then be tested for growth inhibition. The methods described herein may also be used to monitor the effect of these inhibitors on for example down-stream targets of the implicated pathway.

In some embodiments, the methods described herein may be used as a research tool to identify new markers for diagnosis of conditions (such as suspected tumors); to monitor the effect of drugs or candidate drugs on samples such as tumor cells, cell lines, tissues, or organisms; or to uncover new pathways for disease prevention or inhibition (such as oncogenesis and/or tumor suppression).

### Ranges and Numbers

In some cases, an oligonucleotide fragment may comprises one or more epigenetically modified bases, such as (a) one or more epigenetically modified cytosines, (b) one or more epigenetically modified uracils, (c) one or more epigenetically modified thymines, (d) one or more epigenetically modified guanine, (e) one or more epigenetically modified adenines, or (f) any combination thereof.

A nucleic acid sequence may comprise one or more epigenetically modified bases. For example, a nucleic acid sequence may comprise at least about: 1, 2, 3, 4, 5, 6, 7, 8, 9, 10, 11, 12, 13, 14, 15, 16, 17, 18, 19, 20, or more epigenetically modified bases per about 20 basepairs of the nucleic acid sequence. A nucleic acid sequence may comprise about: 1, 2, 3, 4, 5, 6, 7, 8, 9, 10, 11, 12, 13, 14, 15, 16, 17, 18, 19, or 20 epigenetically modified bases per about 20 basepairs of the nucleic acid sequence.

A nucleic acid sequence may comprise one or more epigenetically modified bases. For example, about: 4%, 5%, 6%, 7%, 8%, 9%, 10%, 15%, 20%, 30%, 40%, 50%, 60%, 70%, 80%, or 90% of total bases of a nucleic acid sequence may comprise epigenetically modified bases. In some cases, at least about: 4%, 5%, 6%, 7%, 8%, 9%, 10%, 15%, 20%, 30%, 40%, 50%, 60%, 70%, 80%, or 90% of total bases of a nucleic acid sequence may comprise epigenetically modified bases. In some cases, from about 4% to about 10% of total bases of a nucleic acid sequence may comprise epigenetically modified bases. In some cases, from about 4% to about 6% of total bases of a nucleic acid sequence may comprise epigenetically modified bases. In some cases, from about 4% to about 20% of total bases of a nucleic acid sequence may comprise epigenetically modified bases. In some cases, from about 4% to about 30% of total bases of a nucleic acid sequence may comprise epigenetically modified bases. In some cases, from about 3% to about 30% of total bases of a nucleic acid sequence may comprise epigenetically modified bases. In some cases, from about 30% to about 90% of total bases of a nucleic acid sequence may comprise epigenetically modified bases. In some cases, from about 40% to about 90% of total bases of a nucleic acid sequence may comprise epigenetically modified bases. In some cases, from about 50% to about 90% of total bases of a nucleic acid sequence may comprise epigenetically modified bases. In some cases, from about 60% to about 90% of total bases of a nucleic acid sequence may comprise epigenetically modified bases.

A nucleic acid sequence (in some cases comprising a plurality of epigenetically modified residues) may be enriched. Enrichment of the nucleic acid sequence may comprise amplification such as amplification by polymerase chain reaction (PCR), loop mediated isothermal amplification, nucleic acid sequence based amplification, strand displacement amplification, multiple displacement amplification, rolling circle amplification, ligase chain reaction, helicase dependent amplification, ramification amplification method, or any combination thereof.

In some cases, amplification may comprise at least 2 cycles of amplification. Amplification may comprise at least 3 cycles of amplification. Amplification may comprise at least 4 cycles of amplification. Amplification may comprise at least 5 cycles of amplification. Amplification may comprise at least 6 cycles of amplification. Amplification may comprise at least 7 cycles of amplification. Amplification may comprise at least 8 cycles of amplification. Amplification may comprise at least 9 cycles of amplification. Amplification may comprise at least 10 cycles of amplification. Amplification may comprise at least 11 cycles of amplification. Amplification may comprise at least 12 cycles of amplification. Amplification may comprise at least 13 cycles of amplification. Amplification may comprise at least 14 cycles of amplification. Amplification may comprise at least 15 cycles of amplification. Amplification may comprise at least 20 cycles of amplification. Amplification may comprise at least 25 cycles of amplification. Amplification may comprise at least 30 cycles of amplification.

In some cases, amplification of a given number of cycles produces a plurality of sequence reads that retain a percentage of original sequence length. In some cases, about 90% of the plurality of sequence reads retain at least about 90% of the sequence length. In some cases, about 80% of the plurality of sequence reads retain at least about 90% of the sequence length. In some cases, about 75% of the plurality of sequence reads retain at least about 90% of the sequence length. In some cases, about 95% of the plurality of sequence reads retain at least about 90% of the sequence length. In some cases, about 85% of the plurality of sequence reads retain at least about 90% of the sequence length.

In some cases, about 90% of the plurality of sequence reads retain at least about 85% of the sequence length. In some cases, about 80% of the plurality of sequence reads retain at least about 85% of the sequence length. In some cases, about 75% of the plurality of sequence reads retain at least about 85% of the sequence length. In some cases, about 95% of the plurality of sequence reads retain at least about 85% of the sequence length. In some cases, about 85% of the plurality of sequence reads retain at least about 85% of the sequence length.

In some cases, about 90% of the plurality of sequence reads retain at least about 80% of the sequence length. In some cases, about 80% of the plurality of sequence reads retain at least about 80% of the sequence length. In some cases, about 75% of the plurality of sequence reads retain at least about 80% of the sequence length. In some cases, about 95% of the plurality of sequence reads retain at least about 80% of the sequence length. In some cases, about 85% of the plurality of sequence reads retain at least about 80% of the sequence length.

In some cases, a portion of bases of the substantially complementary strand may base pair with a nucleic acid sequence. In some cases, at least about: 70%, 75%, 80%, 85%, 90%, 95%, or 98% of bases of the substantially complementary strand may base pair with a nucleic acid sequence. In some cases, at least about 70% of bases of the substantially complementary strand may base pair with the nucleic acid sequence. In some cases, at least about 80% of bases of the substantially complementary strand may base pair with the nucleic acid sequence. In some cases, at least about 90% of bases of the substantially complementary strand may base pair with the nucleic acid sequence. In some cases, at least about 95% of bases of the substantially complementary strand may base pair with the nucleic acid sequence. In some cases, at least about 98% of bases of the substantially complementary strand may base pair with the nucleic acid sequence. In some cases, from about 70% to 100% of bases of the substantially complementary strand may base pair with the nucleic acid sequence. In some cases, from about 75% to 100% of bases of the substantially complementary strand may base pair with the nucleic acid sequence. In some cases, from about 80% to 100% of bases of the substantially complementary strand may base pair with the nucleic acid sequence. In some cases, from about 85% to 100% of bases of the substantially complementary strand may base pair with the nucleic acid sequence. In some cases, from about 90% to 100% of bases of the substantially complementary strand may base pair with the nucleic acid sequence. In some case, a substantially complementary strand may hybridize to a nucleic acid sequence under substantially stringent hybridization conditions, such as a substantially high hybridization temperature, a substantially low salt content in a hybridization buffer, or a combination thereof.

In some cases, at least about: 1%, 2%, 3%, 4%, 5%, 10%, 20%, 30%, 40%, 50%, 60%, 70%, 80%, 90% of the bases of a nucleic acid sequence may comprise an epigenetically modified base. In some cases, at least about 1% of the bases of a nucleic acid sequence may comprise an epigenetically modified base.. In some cases, at least about 2% of the bases of a nucleic acid sequence may comprise an epigenetically modified base. In sonic cases, at least about 3% of the bases of a nucleic acid sequence may comprise an epigenetically modified base. In some cases, at least about 4% of the bases of a nucleic acid sequence may comprise an epigenetically modified base. in some cases, at least about 5% of the bases of a nucleic acid sequence may comprise an epigenetically modified base. In some cases, at least about 10% of the bases of a nucleic acid sequence may comprise an epigenetically modified base. In some cases, from about 10% to about 100% of the bases of a nucleic acid sequence may comprise an epigenetically modified base. In some cases, from about 10% to about 90% of the bases of a nucleic acid sequence may comprise an epigenetically modified base. In some cases, from about 5% to about 100% of the bases of a nucleic acid sequence may comprise an epigenetically modified base. In some cases, from about 4% to about 100% of the bases of a nucleic acid sequence may comprise an epigenetically modified base. In some cases, from about 3% to about 100% of the bases of a nucleic acid sequence may comprise an epigenetically modified base.

In some cases, a nucleic acid sequence comprises at least about: 1, 2, 3, 4, 5, 6, 7, 8, 9, 10, 15 or 20 epigenetically modified bases per at least about 20 bases of the nucleic acid sequence. In some cases, a nucleic acid sequence comprises at least about 1 epigenetically modified base per at least about 20 bases of the nucleic acid sequence. In some cases, a nucleic acid sequence comprises at least about 2 epigenetically modified bases per at least about 20 bases of the nucleic acid sequence. In some cases, a nucleic acid sequence comprises at least about 3 epigenetically modified bases per at least about 20 bases of the nucleic acid sequence. In some cases, a nucleic acid sequence comprises at least about 4 epigenetically modified bases per at least about 20 bases of the nucleic acid sequence. In some cases, a nucleic acid sequence comprises at least about 5 epigenetically modified bases per at least about 20 bases of the nucleic acid sequence. In some cases, a nucleic acid sequence comprises at least about 10 epigenetically modified bases per at least about 20 bases of the nucleic acid sequence. In some cases, a nucleic acid sequence comprises from about 1 to about 10 epigenetically modified bases per at least about 20 bases of the nucleic acid sequence. In some cases, a nucleic acid sequence comprises at least from about 3 to about 10 epigenetically modified bases per at least about 20 bases of the nucleic acid sequence. In some cases, a nucleic acid sequence comprises at least from about 4 to about 10 epigenetically modified bases per at least about 20 bases of the nucleic acid sequence. In some cases, a nucleic acid sequence comprises at least from about 5 to about 10 epigenetically modified bases per at least about 20 bases of the nucleic acid sequence.

In some cases, a nucleic acid sequence comprises at least from about 1 to about 3 epigenetically modified bases per at least about 20 bases of a nucleic acid sequence. In some cases, a nucleic acid sequence comprises at least from about 1 to about 4 epigenetically modified bases per at least about 20 bases of a nucleic acid sequence. In some cases, a nucleic acid sequence comprises at least from about 1 to about 5 epigenetically modified bases per at least about 20 bases of a nucleic acid sequence. In some cases, a nucleic acid sequence comprises at least from about 1 to about 8 epigenetically modified bases per at least about 20 bases of a nucleic acid sequence. In some cases, a nucleic acid sequence comprises at least from about 1 to about 10 epigenetically modified bases per at least about 20 bases of a nucleic acid sequence. In some cases, a nucleic acid sequence comprises at least from about 1 to about 15 epigenetically modified bases per at least about 20 bases of a nucleic acid sequence. In some cases, a nucleic acid sequence comprises at least from about 1 to about 20 epigenetically modified bases per at least about 20 bases of a nucleic acid sequence.

### Samples

A sample obtained from a subject can comprise tissue, cells, cell fragments, cell organelles, nucleic acids, genes, gene fragments, expression products, gene expression products, gene expression product fragments or any combination thereof. A sample can be heterogeneous or homogenous. A sample can comprise blood, urine, cerebrospinal fluid, seminal fluid, saliva, sputum, stool, lymph fluid, tissue, mucus, or any combination thereof. A sample can be a tissue-specific sample such as a sample obtained from a reproductive tissue (such as a sperm or an egg), thyroid, skin, heart, lung, kidney, breast, pancreas, liver, muscle, smooth muscle, bladder, gall bladder, colon, intestine, brain, esophagus, prostate, or any combination thereof.

A sample of the present disclosure can be obtained by various methods, such as, for example, fine needle aspiration (FNA), core needle biopsy, vacuum assisted biopsy, incisional biopsy, excisional biopsy, core biopsy, punch biopsy, shave biopsy, skin biopsy, or any combination thereof.

A sample may be obtained from a subject by another individual or entity, such as a healthcare (or medical) professional or robot. A medical professional can include a physician, nurse, medical technician or other. In some cases, a physician may be a specialist, such as an oncologist, surgeon, or endocrinologist. A medical technician may be a specialist, such as a cytologist, phlebotomist, radiologist, pulmonologist or others. medical professional may obtain a sample from a subject for testing or refer the subject to a testing center or laboratory for the submission of the sample. The medical professional may indicate to the testing center or laboratory the appropriate test or assay to perform on the sample, such as methods of the present disclosure including determining gene sequence data, gene expression levels, sequence variant data, or any combination thereof.

In some cases, a medical professional need not be involved in the initial diagnosis of a condition or a disease or the initial sample acquisition. An individual, such as the subject, may alternatively obtain a sample through the use of an over the counter kit. The kit may contain collection unit or device for obtaining the sample as described herein, a storage unit for storing the sample ahead of sample analysis, and instructions for use of the kit.

Epigenetic modifications may be monitored over time. Monitoring epigenetic modification over time may include monitoring changes in a presence of an epigenetic modification, a level of an epigenetic modification, a pattern of an epigenetic modification. Monitoring may include monitoring an efficacy of a therapeutic, monitoring a progression of a disease, monitoring a regression of a disease, monitoring a risk or likelihood of developing a disease, monitoring a mortality prediction or biological age, or any combination thereof. A sample can be obtained a) pre-operatively, b) post-operatively, c) after a disease diagnosis, d) during routine screening following remission or cure of a disease, e) when a subject may be suspected of having a disease, f) during a routine office visit or clinical screen, g) following the request of a medical professional, or any combination thereof. Multiple samples at separate times can be obtained from the same subject, such as before treatment for a disease commences and after treatment ends, such as monitoring a subject over a time course. Multiple samples can be obtained from a subject at separate times to monitor the absence or presence of disease progression, regression, or remission in the subject.

### Conditions or Diseases

A condition or a disease, as disclosed herein, can include a cancer, a neurological disorder, or an autoimmune disease.

In some cases, a disease or condition may comprise a neurological disorder, In some cases, a neurological disorder may comprise Acquired Epileptiform Aphasia, Acute Disseminated Encephalomyelitis, Adrenoleukodystrophy, Agenesis of the corpus callosum, Agnosia, Aicardi syndrome, Alexander disease, Alpers' disease, Alternating hemiplegia, Alzheimer's disease, Amyotrophic lateral sclerosis (see Motor Neuron Disease), Anencephaly, Angelman syndrome, Angiomatosis, Anoxia, Aphasia, Apraxia, Arachnoid cysts, Arachnoiditis, Arnold-Chiari malformation, Arteriovenous malformation, Asperger's syndrome, Ataxia Telangiectasia, Attention Deficit Hyperactivity Disorder, Autism, Auditory processing disorder, Autonomic Dysfunction, Back Pain, Batten disease, Behcet's disease, Bell's palsy, Benign Essential Blepharospasm, Benign Focal Amyotrophy, Benign Intracranial Hypertension, Bilateral frontoparietal polymicrogyria, Binswanger's disease, Blepharospasm, Bloch-Sulzberger syndrome, Brachial plexus injury, Brain abscess, Brain damage, Brain injury, Brain tumor, Brown-Sequard syndrome, Canavan disease, Carpal tunnel syndrome (CTS), Causalgia, Central pain syndrome, Central pontine myelinolysis, Centronuclear myopathy, Cephalic disorder, Cerebral aneurysm, Cerebral arteriosclerosis, Cerebral atrophy, Cerebral gigantism, Cerebral palsy, Charcot-Marie-Tooth disease, Chiari malformation, Chorea, Chronic inflammatory demyelinating polyneuropathy (CIDP), Chronic pain, Chronic regional pain syndrome, Coffin Lowry syndrome, Coma, including Persistent Vegetative State, Congenital facial diplegia, Corticobasal degeneration, Cranial arteritis, Craniosynostosis, Creutzfeldt-Jakob disease, Cumulative trauma disorders, Cushing's syndrome, Cytomegalic inclusion body disease (CIBD), Cytomegalovirus Infection, Dandy-Walker syndrome, Dawson disease, De Morsier's syndrome, Dejerine-Klumpke palsy, Dejerine-Sottas disease, Delayed sleep phase syndrome, Dementia, Dermatomyositis, Neurological Dyspraxia, Diabetic neuropathy, Diffuse sclerosis, Dysautonomia, Dyscalculia, Dysgraphia, Dyslexia, Dystonia, Early infantile epileptic encephalopathy, Empty sella syndrome, Encephalitis, Encephalocele, Encephalotrigeminal angiomatosis, Encopresis, Epilepsy, Erb's palsy, Erythromelalgia, Essential tremor, Fabry's disease, Fahr's syndrome, Fainting, Familial spastic paralysis, Febrile seizures, Fisher syndrome, Friedreich's ataxia, FART Syndrome, Gaucher's disease, Gerstmann's syndrome, Giant cell arteritis, Giant cell inclusion disease, Globoid cell Leukodystrophy, Gray matter heterotopia, Guillain-Barre syndrome, HTLV-1 associated myelopathy, Hallervorden-Spatz disease, Head injury, Headache, Hemifacial Spasm, Hereditary Spastic Paraplegia, Heredopathia atactica polyneuritiformis, Herpes zoster oticus, Herpes zoster, Hirayama syndrome, Holoprosencephaly, Huntington's disease, Hydranencephaly, Hydrocephalus, Hypercortisolism, Hypoxia, Immune-Mediated encephalomyelitis, Inclusion body myositis, Incontinentia pigmenti, Infantile phytanic acid storage disease, Infantile Refsum disease, Infantile spasms, Inflammatory myopathy, Intracranial cyst, Intracranial hypertension, Joubert syndrome, Kearns-Sayre syndrome, Kennedy disease, Kinsbourne syndrome, Klippel Feil syndrome,

Krabbe disease, Kugelberg-Welander disease, Kuru, Lafora disease, Lambert-Eaton myasthenic syndrome, Landau-Kleffner syndrome, Lateral medullary (Wallenberg) syndrome, Learning disabilities, Leigh's disease, Lennox-Gastaut syndrome, Lesch-Nyhan syndrome, Leukodystrophy, Lewy body dementia, Lissencephaly, Locked-In syndrome, Lou Gehrig's disease, Lumbar disc disease, Lyme disease-Neurological Sequelae, Machado-Joseph disease (Spinocerebellar ataxia type 3), Macrencephaly, Maple Syrup Urine Disease, Megalencephaly, Melkersson-Rosenthal syndrome, Menieres disease, Meningitis, Menkes disease, Metachromatic leukodystrophy, Microcephaly, Migraine, Miller Fisher syndrome, Mini-Strokes, Mitochondrial Myopathies, Mobius syndrome, Monomelic amyotrophy, Motor Neuron Disease, Motor skills disorder, Moyamoya disease, Mucopolysaccharidoses, Multi-Infarct Dementia, Multifocal motor neuropathy, Multiple sclerosis, Multiple system atrophy, Muscular dystrophy, Myalgic encephalomyelitis, Myasthenia gravis, Myelinoclastic diffuse sclerosis, Myoclonic Encephalopathy of infants, Myoclonus, Myopathy, Myotubular myopathy, Myotonia congenita,Narcolepsy, Neurofibromatosis, Neuroleptic malignant syndrome, Neurological manifestations of AIDS, Neurological sequelae of lupus, Neuromyotonia, Neuronal ceroid lipofuscinosis, Neuronal migration disorders, Niemann-Pick disease, Non 24-hour sleep-wake syndrome, Nonverbal learning disorder, O'Sullivan-McLeod syndrome, Occipital Neuralgia, Occult Spinal Dysraphism Sequence, Ohtahara syndrome, Olivopontocerebellar atrophy, Opsoclonus myoclonus syndrome, Optic neuritis, Orthostatic Hypotension, Overuse syndrome, Palinopsia, Paresthesia, Parkinson's disease, Paramyotonia Congenita, Paraneoplastic diseases, Paroxysmal attacks, Parry-Romberg syndrome, Rombergs Syndrome, Pelizaeus-Merzbacher disease, Periodic Paralyses, Peripheral neuropathy, Persistent Vegetative State, Pervasive neurological disorders, Photic sneeze reflex, Phytanic Acid Storage disease, Pick's disease, Pinched Nerve, Pituitary Tumors, PMG, Polio, Polymicrogyria, Polymyositis, Porencephaly, Post-Polio syndrome, Postherpetic Neuralgia (PHN), Postinfectious Encephalomyelitis, Postural Hypotension, Prader-Willi syndrome, Primary Lateral Sclerosis, Prion diseases, Progressive Hemifacial Atrophy also known as Rombergs Syndrome, Progressive multifocal leukoencephalopathy, Progressive Sclerosing Poliodystrophy, Progressive Supranuclear Palsy, Pseudotumor cerebri, Ramsay-Hunt syndrome (Type I and Type II), Rasmussen's encephalitis, Reflex sympathetic dystrophy syndrome, Refsum disease, Repetitive motion disorders, Repetitive stress injury, Restless legs syndrome, Retrovirus-associated myelopathy, Rett syndrome, Reye's syndrome, Rombergs Syndrome, Rabies, Saint Vitus dance, Sandhoff disease, Schytsophrenia, Schilder's disease, Schizencephaly, Sensory Integration Dysfunction, Septo-optic dysplasia, Shaken baby syndrome, Shingles, Shy-Drager syndrome, Sjogren's syndrome, Sleep apnea, Sleeping sickness, Snatiation, Sotos syndrome, Spasticity, Spina bifida, Spinal cord injury, Spinal cord tumors, Spinal muscular atrophy, Spinal stenosis, Steele-Richardson-Olszewski syndrome, see Progressive Supranuclear Palsy, Spinocerebellar ataxia, Stiff-person syndrome, Stroke, Sturge-Weber syndrome, Subacute sclerosing panencephalitis, Subcortical arteriosclerotic encephalopathy, Superficial siderosis, Sydenham's chorea, Syncope, Synesthesia, Syringomyelia, Tardive dyskinesia, Tay-Sachs disease, Temporal arteritis, Tethered spinal cord syndrome, Thomsen disease, Thoracic outlet syndrome, Tic Douloureux, Todd's paralysis, Tourette syndrome, Transient ischemic attack, Transmissible spongiform encephalopathies, Transverse myelitis, Traumatic brain injury, Tremor, Trigeminal neuralgia, Tropical spastic paraparesis, Trypanosomiasis, Tuberous sclerosis, Vasculitis including temporal arteritis, Von Hippel-Lindau disease (VEIL), Viliuisk Encephalomyelitis (VE), Wallenberg's syndrome, Werdnig-Hoffman disease, West syndrome, Whiplash, Williams syndrome, Wilson's disease, X-Linked Spinal and Bulbar Muscular Atrophy, and Zellweger syndrome. Neurological conditions can comprise movement disorders, for example multiple system atrophy (MSA).

In some cases, a disease or condition may comprise an autoimmune disease. In some cases, an autoimmune disease may comprise acute disseminated encephalomyelitis (ADEM), acute necrotizing hemorrhagic leukoencephalitis, Addison's disease, agammaglobulinemia, allergic asthma, allergic rhinitis, alopecia areata, amyloidosis, ankylosing spondylitis, anti-GBM/anti-TBM nephritis, antiphospholipid syndrome (APS), autoimmune aplastic anemia, autoimmune dysautonomia, autoimmune hepatitius, autoimmune hyperlipidemia, autoimmune immunodeficiency, autoimmune inner ear disease (MED), autoimmune myocarditis, autoimmune pancreatitis, autoimmune retinopathy, autoimmune thrombocytopenic purpura (ATP), autoimmune thyroid disease, axonal & neuronal neuropathies. Balo disease, Behcet's disease, bullous pemphigoid, cardiomyopathy, Castlemen disease, celiac sprue (non-tropical), Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, congenital heart block, coxsackie myocarditis. CREST disease, essential mixed cryoglobulinemia, demyelinating neuropathies, dermatomyositis, Devic's disease (neuromyelitis optica), discoid lupus, Dressler's syndrome, endometriosis, eosinophillic fasciitis, erythema nodosum, experimental allergic encephalomyelitis, Evan's syndrome, fibromyalgia., fibrosing alveolitis, giant cell arteritis (temporal arteritis), glomerulonephritis, Goodpasture's syndrome, Grave's disease, Guillain-Barre syndrome. Hashimoto's encephalitis, Hashimoto's thyroiditis, hemolytic anemia, Henock-Schoniein purpura, herpes gestationis, hypogammaglobulinemia, idiopathic thrombocytopenic purpura (ITP), IgA nephropathy, immmunoregulatory lipoproteins, inclusion body myositis, insulin-dependent diabetes (type 1), interstitial cystitis, juvenile arthritis, juvenile diabetes, Kawasaki syndrome, Lambert-Eaton syndrome, leukocytoclastic vasculitis, lichen planus, lichen sclerosus, ligneous conjunctivitis, linear IgA disease (LAD), Lupus (SLE), Lynie disease, Meniere's disease, microscopic polyangitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune. Neuropsychiatric Disorders Associated with Streptococcus), paraneoplastic cerebellar degeneration, paroxysmal nocturnal hernoglobinuria (PNH), Parry Romberg syndrome, Parsonnage-Turner syndrome, pars plantis (peripheral uveitis), pemphigus, peripheral neuropathy, perivenous encephalomyelitis, pernicious anemia, POEMS syndrome, polyarteritis nodosa, type I, II & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasis, Raynaud's phenomena, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Slogren's syndrome, sperm and testicular autoimmunity, stiff person syndrome, subacute bacterial endocarditis (SBE), sympathetic ophthalmia, Takayasu's arteritis, temporal arteritis/giant cell arteries, thrombocytopenic purpura (TPP), Tolosa-Hunt syndrome, transverse myelitis, ulcerative colitis, undifferentiated connective tissue disease (UCTD), uveitis, vasculitis, vesiculobullous dermatosis, vitiligo or Wegener's granulomatosis or , chronic active hepatitis, primary biliary cirrhosis, cadilated cardiomyopathy, myocarditis, autoimmune polyendocrine syndrome type I (APS-I), cystic fibrosis vasculitides, acquired hypoparathyroidism, coronary artery disease, pemphigus foliaceus, pemphigus vulgaris, Rasmussen encephalitis, autoimmune gastritis, insulin hypoglycemic syndrome (Hirata disease), Type B insulin resistance, acanthosis, systemic lupus erythematosus (SLE), pernicious anemia, treatment-resistant Lyme arthritis, polyneuropathy, demyelinating diseases, atopic dermatitis, autoimmune hypothyroidism, vitiligo, thyroid associated ophthalmopathy, autoimmune coeliac disease, ACTH deficiency, dermatomyositis, Sjogren syndrome, systemic sclerosis, progressive systemic sclerosis, morphea, primary antiphospholipid syndrome, chronic idiopathic urticaria, connective tissue syndromes, necrotizing and crescentic glomerulonephritis (NCGN), systemic vasculitis, Raynaud syndrome, chronic liver disease, visceral leishmaniasis, autoimmune C1 deficiency, membrane proliferative glomerulonephritis (MPGN), prolonged coagulation time, immunodeficiency, atherosclerosis, neuronopathy, paraneoplastic pemphigus, paraneoplastic stiff man syndrome, paraneoplastic encephalomyelitis, subacute autonomic neuropathy, cancer-associated retinopathy, paraneoplastic opsoclonus myoclonus ataxia, lower motor neuron syndrome and Lambert-Eaton myasthenic syndrome.

In some cases, a disease or a condition may comprise AIDS, anthrax, botulism, brucellosis, chancroid, chlamydial infection, cholera, coccidioidomycosis, cryptosporidiosis, cyclosporiasis, dipheheria, ehrlichiosis, arboviral encephalitis, enterohemorrhagic Escherichia coli, giardiasis, gonorrhea, dengue fever, haemophilus influenza, Hansen's disease (Leprosy), hantavirus pulmonary syndrome, hemolytic uremic syndrome, hepatitis A, hepatitis B, hepatitis C, human immunodeficiency virus, legionellosis, listeriosis, lyme disease, malaria, measles. Meningococcal disease, mumps, pertussis (whooping cough), plague, paralytic poliomyelitis, psittacosis, Q fever, rabies, rocky mountain spotted fever, rubella, congenital rubella syndrome (SARS), shigellosis, smallpox, streptococcal disease (invasive group A), streptococcal toxic shock syndrome, streptococcus pneumonia, syphilis, tetanus, toxic shock syndrome, trichinosis, tuberculosis, tularemia, typhoid fever, vancomycin intermediate resistant staphylocossus aureus, varicella, yellow fever, variant Creutzfeldt-Jakob disease (vCJD), Eblola hemorrhagic fever, Echinococcosis, Hendra virus infection, human monkeypox, influenza. A, H5N1, lassa fever, Margurg hemorrhagic fever, Nipah virus, O'nyong fever, Rift valley fever, Venezuelan equine encephalitis and West Nile virus.

In some cases, a disease or condition may comprise a cancer. In some cases, a cancer may comprise thyroid cancer, adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's disease, cervical cancer, childhood. Non-Hodgkin's lymphoma, lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, uterine cancer (e.g. uterine sarcoma), vaginal cancer, vulvar cancer, or Waldenstrom's macroglobulinaemia.

A condition or a disease, as disclosed herein, can include hyperproliferative disorders. Malignant hyperproliferative disorders can be stratified into risk groups, such as a low risk group and a medium-to-high risk group. Hyperproliferative disorders can include but may not be limited to cancers, hyperplasia, or neoplasia. In some cases, the hyperproliferative cancer can be breast cancer such as a ductal carcinoma in duct tissue of a mammary gland, medullary carcinomas, colloid carcinomas, tubular carcinomas, and inflammatory breast cancer; ovarian cancer, including epithelial ovarian tumors such as adenocarcinoma in the ovary and an adenocarcinoma. that has migrated from the ovary into the abdominal cavity; uterine cancer; cervical cancer such as adenocarcinoma in the cervix epithelial including squamous cell carcinoma and adenocarcinomas; prostate cancer, such as a prostate cancer selected from the following: an adenocarcinoma or an adenocarcinoma that has migrated to the bone; pancreatic cancer such as epithelioid carcinoma in the pancreatic duct tissue and an adenocarcinoma in a pancreatic duct; bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers; leukemia such as acute myeloid leukemia (AMU, acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CMI), mastocytosis, chronic lymphocytic leukemia (CLL), multiple myeloma (MM), and myelodysplastic syndrome (MDS); bone cancer; lung cancer such as non-small cell lung cancer (NSCLC), which may be divided into squamous cell carcinomas, adenocarcinomas, and large cell undifferentiated carcinomas, and small cell lung cancer; skin cancer such as basal cell carcinoma, melanoma, squamous cell carcinoma and actinic keratosis, which may be a skin condition that sometimes develops into squamous cell carcinoma; eye retinoblastoma; cutaneous or intraocular (eye) melanoma; primary liver cancer (cancer that begins in the liver); kidney cancer; autoimmune deficiency syndrome (AIDS)-related lymphoma such as diffuse large B-cell lymphoma, B-cell immunoblastic lymphoma and small non-cleaved cell lymphoma; Kaposi's Sarcoma; viral-induced cancers including hepatitis B virus (HM), hepatitis C virus (HCV), and hepatocellular carcinoma; human lymphotropic virus-type I (HBV) and adult T-cell leukemialymphoma; and human papilloma virus (HPV) and cervical cancer; central nervous system (CNS) cancers such as primary brain tumor, which includes gliomas (astrocytoma, anaplastic astrocytoma, or glioblastoma multiforme), oligodendrogliomas, ependymomas, meningiomas, lymphomas, schwannomas, and medulloblastomas; peripheral nervous system (PNS) cancers such as acoustic neuromas and malignant peripheral nerve sheath tumors (MPNST) including neurofibromas and schwannomas, malignant fibrous cytomas, malignant fibrous histiocytomas, malignant meningiomas, malignant mesotheliomas, and malignant mixed Müllerian tumors; oral cavity and oropharyngeal cancer such as hypopharyngeal cancer, laryngeal cancer, nasopharyngeal cancer, and oropharyngeal cancer; stomach cancer such as lymphomas, gastric stromal tumors, and carcinoid tumors; testicular cancer such as germ cell tumors (GCTs), which include seminomas and nonseminomas, and gonadal stromal tumors, which include Leydig cell tumors and Sertoli cell tumors; thymus cancer such as to thymomas, thymic carcinomas, Hodgkin disease, non-Hodgkin lymphomas carcinoids or carcinoid tumors; rectal cancer; and colon cancer. In some cases, the diseases stratified, classified, characterized, or diagnosed by the methods of the present disclosure include but may not be limited to thyroid disorders such as for example benign thyroid disorders including but not limited to follicular adenomas, Hurthle cell adenomas, lymphocytic thyroiditis, and thyroid hyperplasia. In some cases, the diseases stratified, classified, characterized, or diagnosed by the methods of the present disclosure include but may not be limited to malignant thyroid disorders such as for example follicular carcinomas, follicular variant of papillary thyroid carcinomas, medullary carcinomas, and papillary carcinomas.

Conditions or diseases of the present disclosure can include a genetic disorder. A genetic disorder may be an illness caused by abnormalities in genes or chromosomes. Genetic disorders can be grouped into two categories: single gene disorders and multifactorial and polygenic (complex) disorders. A single gene disorder can be the result of a single mutated gene. Inheriting a single gene disorder can include but not be limited to autosomal dominant, autosomal recessive, X-linked dominant, X-linked recessive, Y-linked and mitochondrial inheritance. Only one mutated copy of the gene can be necessary for a person to be affected by an autosomal dominant disorder. Examples of autosomal dominant type of disorder can include but may not be limited to Huntington's disease, Neurofibromatosis 1, Marfan Syndrome, Hereditary nonpolyposis colorectal cancer, or Hereditary multiple exostoses, In autosomal recessive disorders, two copies of the gene must be mutated for a subject to be affected by an autosomal recessive disorder. Examples of this type of disorder can include but may not be limited to cystic fibrosis, sickle-cell disease (also partial sickle-cell disease), Tay-Sachs disease, Niemann-Pick disease, or spinal muscular atrophy. X-linked dominant disorders are caused by mutations in genes on the X chromosome such as X-linked hypophosphatemic rickets, Some X-linked dominant conditions such as Rett syndrome, Incontinentia Pigmenti type 2 and Aicardi Syndrome can be fatal. X-linked recessive disorders are also caused by mutations in genes on the X chromosome, Examples of this type of disorder can include but are not limited to Hemophilia A, Duchenne muscular dystrophy, red-green color blindness, muscular dystrophy and Androgenetic alopecia. Y-linked disorders are caused by mutations on the Y chromosome, Examples can include but are riot limited to Male Infertility and hypertrichosis pinnae. The genetic disorder of mitochondrial inheritance, also known as maternal inheritance, can apply to genes in mitochondrial DNA such as in Leber's Hereditary Optic Neuropathy.

Genetic disorders may also be complex, multifactorial or polygenic. Polygenic genetic disorders can be associated with the effects of multiple genes in combination with lifestyle and environmental factors. Although complex genetic disorders can cluster in families, they do not have a clear-cut pattern of inheritance. Multifactorial or polygenic disorders can include heart disease, diabetes, asthma, autism, autoimmune diseases such as multiple sclerosis, cancers, ciliopathies, cleft palate, hypertension, inflammatory bowel disease, mental retardation or obesity.

Other genetic disorders can include but may not be limited to 1p36 deletion syndrome, 21-hydroxyla.se deficiency, 22q11.2 deletion syndrome, aceruloplasminemia, achondrogenesis, type achondroplasia, acute intermittent porphyria, adenylosuccinate lyase deficiency, Adrenoleukodystrophy, Alexander disease, alkaptonuria, alpha-I antitrypsin deficiency, Alstrom syndrome, Alzheimer's disease (type 1, 2, 3, and 4), Amelogenesis Imperfecta, amyotrophic lateral sclerosis, Amyotrophic lateral sclerosis type 2, Amyotrophic lateral sclerosis type 4, amyotrophic lateral sclerosis type 4, androgen insensitivity syndrome, Anemia, Angelman syndrome, Apert syndrome, ataxia-telangiectasia, Beare-Stevenson cutis gyrata syndrome, Benjamin syndrome, beta thalassemia, biotimidase deficiency, Birt-Hogg-Dube syndrome, bladder cancer, Bloom syndrome, Bone diseases, breast cancer, Camptomelic dysplasia, Canavan disease, Cancer, Celiac Disease, Chronic Granulomatous Disorder (CGD), Charcot-Marie-Tooth disease, Charcot-Marie-Tooth disease Type 1, Charcot-Marie-Tooth disease Type 4, Charcot-Marie-Tooth disease Type 2, Charcot-Marie-Tooth disease Type 4, Cockayne syndrome, Coffin-Lowry syndrome, collagenopathy types II and XI, Colorectal Cancer, Congenital absence of the vas deferens, congenital bilateral absence of vas deferens, congenital diabetes, congenital erythropoietic porphyria, Congenital heart disease, congenital hypothyroidism, Connective tissue disease, Cowden syndrome, Cri du chat syndrome, Crohn's disease, fibrostenosing, Crouzon syndrome, Crouzonodermoskeletal syndrome, cystic fibrosis, De Grouchy Syndrome, Degenerative nerve diseases, Dent's disease, developmental disabilities, DiGeorge syndrome, Distal spinal muscular atrophy type V, Down syndrome, Dwarfism, Ehlers-Danlos syndrome, Ehlers-Danlos syndrome arthrochalasia type, Ehlers-Danlos syndrome classical type, Ehlers-Danlos syndrome dermatosparaxis type, Ehlers-Danlos syndrome kyphoscoliosis type, vascular type, erythropoietic protoporphyria, Fabry's disease, Facial injuries and disorders, factor V Leiden thrombophilia, familial adenomatous polyposis, familial dysautonomia, fanconi anemia, FG syndrome, fragile X syndrome, Friedreich ataxia, Friedreich's ataxia, G6PD deficiency, galactosemia, Gaucher's disease (type 1, 2, and 3), Genetic brain disorders, Glycine encephalopathy, Haemochromatosis type 2, Haemochromatosis type 4, Harlequin Ichthyosis, Head and brain malformations, Hearing disorders and deafness, Hearing problems in children, hemochromatosis (neonatal, type 2 and type 3), hemophilia, hepatoerythropoietic porphyria, hereditary coproporphyria, Hereditary Multiple Exostoses, hereditary neuropathy with liability to pressure palsies, hereditary nonpolyposis colorectal cancer, homocystinuria, Huntington's disease, Hutchinson Gilford Progeria Syndrome, hyperoxaluria, primary, hyperphenylalaninemia, hypochondrogenesis, hypochondroplasia, idic15, incontinentia pigmenti, infantile Gaucher disease, infantile-onset ascending hereditary spastic paralysis, infertility, Jackson-Weiss syndrome, Joubert syndrome, Juvenile Primary Lateral Sclerosis, Kennedy disease, Klinefelter syndrome, Kniest dysplasia, Krabbe disease, Learning disability, Lesch-Nyhan syndrome, Leukodystrophies, Li-Fraumeni syndrome, lipoprotein lipase deficiency, familial, Male genital disorders, Marfan syndrome, McCune-Albright syndrome, McLeod syndrome, Mediterranean fever, familial, Menkes disease, Menkes syndrome, Metabolic disorders, methemoglobinemia beta-globin type, Methemoglobinemia congenital methaemoglobinaemia, methylmalonic acidemia, Micro syndrome, Microcephaly, Movement disorders, Mowat-Wilson syndrome, Mucopolysaccharidosis (MPS I), Muenke syndrome. Muscular dystrophy. Muscular dystrophy, Duchenne and Becker type, muscular dystrophy, Duchenne and Becker types, myotonic dystrophy, Myotonic dystrophy type 1 and type 2, Neonatal hemochromatosis, neurofibromatosis, neurofibromatosis 1, neurofibromatosis 2, Neurofibromatosis type I, neurofibromatosis type II, Neurologic diseases, Neuromuscular disorders, Niemann-Pick disease, Nonketotic hyperglycinemia, nonsyndromic deafness, Nonsyndromic deafness autosomal recessive, Noonan syndrome, osteogenesis imperfecta (type I and type III), otospondylomegaepiphyseal dysplasia, pantothenate kinase-associated neurodegeneration, Patau Syndrome (Trisomy 13), Pendred syndrome, Peutz-Jeghers syndrome, Pfeiffer syndrome, phenylketonuria, porphyria, porphyria cutanea tarda, Prader-Willi syndrome, primary pulmonary hypertension, prion disease, Progeria, propionic acidemia, protein C deficiency, protein S deficiency, pseudo-Gaucher disease, pseudoxanthoma elasticum, Retinal disorders, retinoblastoma, retinoblastoma FA—Friedreich ataxia, Rett syndrome, Rubinstein-Taybi syndrome, Sandhoff disease, sensory and autonomic neuropathy type III, sickle cell anemia, skeletal muscle regeneration, Skin pigmentation disorders, Smith Lemli Opitz Syndrome, Speech and communication disorders, spinal muscular atrophy, spinal-bulbar muscular atrophy, spinocerebellar ataxia, spondyloepimetaphyseal dysplasia, Strudwick type, spondyloepiphyseal dysplasia congenita, Stickler syndrome, Stickler syndrome COL2A1, Tay-Sachs disease, tetrahydrobiopierin deficiency, thanatophoric dysplasia, thiamine-responsive megaloblastic anemia with diabetes mellitus and sensorineural deafness, Thyroid disease, Tourette's Syndrome, Treacher Collins syndrome, triple X syndrome, tuberous sclerosis, Turner syndrome, Usher syndrome, variegate porphyria, von Hippel-Lindau disease, Waardenburg syndrome, Weissenbacher-Zweymüller syndrome, Wilson disease, Wolf-Hirschhorn syndrome, Xeroderma Pigmentosum, X-linked severe combined immunodeficiency, X-linked sideroblastic anemia, or X-linked spinal-bulbar muscle atrophy.

### Kits

A kit may include a label, a substrate (such as a solid support), a control nucleic acid sequence, a container, an enzyme or fragment thereof, instructions for use, or any combination thereof. A control nucleic acid sequence may be associated with the substrate. A control nucleic acid sequence may not be associated with a substrate and the kit may include instructions for associating the control nucleic acid sequence with the substrate.

A kit may be a general kit for all tissue samples or disease types. A kit may be a specific kit for a specific tissue sample, such as a plasma sample, a blood sample, a serum sample, a buccal sample, or a urine sample. A kit may be a specific kit for a specific disease such as cancer.

A kit may provide periodic updates of a database of references or analysis software that compute a result of the method. A kit may provide software to automate one or more aspects of a method, such as a comparison to a reference to provide a result or to provide a summary of a result that may be be reported or displayed or downloaded by a medical professional and/or entered into a database. A result or a summary of results may include any of the results disclosed herein, including recommendations of treatment options for subject and a risk occurrence of a disease or condition.

A kit may provide a unit or device for obtaining a sample from a subject (e.g., a device with a needle coupled to an aspirator).

A kit may provide instructions for performing methods as disclosed herein, and include all necessary buffers and reagents for hybridizing, sequencing, amplifying, associating, extending, or combination thereof. A kit may include instructions for analyzing a result.

An informational material of a kit may comprise printed matter, e.g., a printed text, drawing, and/or photograph, e.g., a. label or printed sheet. An information material may comprise Braille, computer readable material, video recording, or audio recording. In some cases, the informational material of the kit may include contact information, e.g., a. physical address, email address, website, or telephone number, where a user of the kit can obtain substantive information about a compound described herein and/or its use in the methods described herein. Informational material may be provided in any combination of formats.

A kit may include a package, such as a fiber-based package, a cardboard package, or a polymeric package, such as a styrofoam box. A package may be configured so as to substantially maintain a temperature differential between an interior and an exterior. In some cases, it may provide insulating properties to keep one or more components of a kit at a preselected temperature for a preselected time. A kit may include one or more containers for a composition containing a compound(s) described herein. In some embodiments, a kit may contain separate containers (such as two separate containers for two components of a kit), dividers or compartments for one or more components, and informational material. For example, a kit component may be contained in a bottle, a vial, or a syringe, and informational material may be contained in a plastic sleeve or a packet. In other embodiments, separate components of a kit may be contained within a single, undivided container. For example, a kit component may be contained in a bottle, a vial or a syringe that has attached thereto the informational material in the form of a label. In some embodiments, a kit may include a plurality (e.g., a pack) of individual containers, each containing one or more unit dosage forms (e.g., a dosage form described herein) of a component described herein. For example, the kit may include a plurality of syringes, ampules, foil packets, or blister packs, each containing a single unit dose of a kit component described herein. Containers of a kit may be air tight, waterproof (e.g., impermeable to changes in moisture or evaporation), and/or light-tight. A kit may include a device suitable for administration of the component, e.g., a syringe, inhalant, pipette, forceps, measured spoon, dropper (e.g,, eye dropper), swab (e.g., a cotton swab or wooden swab), or any such delivery device. In a preferred embodiment, the device may be a medical implant device, e.g., packaged for surgical insertion.

A basic research business, a disease diagnostic business, a molecular profiling business, a pharmaceutical business, or any other business associated with patient healthcare may provide a kit for performing the methods described herein.

### Computer Control Systems

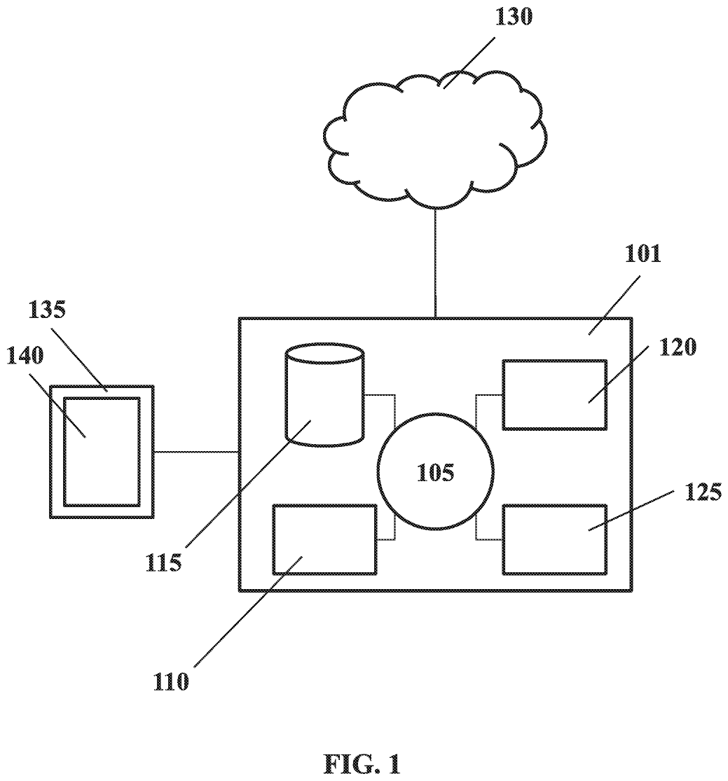

The present disclosure provides computer control systems that are programmed to implement methods of the disclosure. FIG. 1 shows a computer system 101 that is programmed or otherwise configured to interface with a sequence library, a sequencer, a PCR machine, an apparatus that is configured to sequence or amplify an oligonucleotide, a substrate, or any combination thereof. The computer system 101 can regulate various aspects of substrate enrichment of the present disclosure, such as, for example, conditions for washing such as the number of washes, the type of buffer used. The computer system 101 can regulate amplification conditions, labeling conditions, sequencing conditions, such as buffer types, temperatures, or time periods of incubation. The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 can also include memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 can be in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some cases is a telecommunication and/or data network. The network 130 can include one or more computer servers, which can enable distributed computing, such as cloud computing. The network 130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The storage unit 115 can store files, such as drivers, libraries and saved programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example, one or more results (immediate results or archived results from a previous experiment), one or more user inputs, reference values from a library or database, or a combination thereof. Examples of UT's include, without limitation, a graphical user interface (GUI) and web-based user interface.

Methods and systems of the present disclosure can be implemented by way of one or more algorithms. An algorithm can be implemented by way of software upon execution by the central processing unit 105. The algorithm can, for example, determine optimized conditions via supervised learning to optimize conditions such as a buffer type, a buffer concentration, a temperature, an incubation period. Conditions may be optimized for an oligonucleotide fragment, such as an oligonucleotide fragment having a particular number of epigenetic modifications or a particular length of sequence.

### EMBODIMENTS

An aspect of the present disclosure provides a method. In some cases, the method may comprise (a) associating a label with an epigenetically modified base of a nucleic acid sequence to form a labeled nucleic acid sequence; (b) hybridizing a substantially complementary strand to the labeled nucleic acid sequence; and (c) amplifying the substantially complementary strand in a reaction in which the labeled nucleic acid sequence may be substantially not present.

Another aspect of the present disclosure provides a method. In some cases, the method may comprise (a) hybridizing a substantially complementary strand to a nucleic acid sequence comprising an epigenetically modified base; (b) associating a label with the epigenetically modified base of a nucleic acid sequence to form a labeled nucleic acid sequence; and (c) amplifying the substantially complementary strand in a reaction in which the labeled nucleic acid sequence may be substantially not present.

In some cases, the label may be associated with a substrate. In some cases, the substrate may comprise a bead. In some cases, the bead may be a magnetic bead. In some cases, the substrate may comprise an array.

In some cases, the substantially complimentary strand may be shorter in length than the labeled nucleic acid sequence. In some cases, the substantially complimentary strand may be elongated before the amplifying. In some cases, hybridizing may comprise hybridizing at least two substantially complementary strands to the labeled nucleic acid sequence. In some cases, the method may comprise ligating the at least two substantially complementary strands.

In some cases, the labeled nucleic acid sequence may comprise an adapter sequence. In some cases, hybridizing may comprise hybridizing at least a portion of the substantially complimentary strand to the adapter sequence. In some cases, the nucleic acid sequence may comprise a first barcode. In some cases, the nucleic acid sequence may comprise a second barcode. In some cases, the first barcode may be a unique barcode and the second barcode may be a sample barcode.

In some cases, the epigenetically modified base of the nucleic acid sequence may be a hydroxymethylated base (hmB). In some cases, the hmB may be 5-hydroxymethylated base (5-hmB). In some cases, the 5-hmB may be a 5-hydroxymethylated cytosine (5-hmC). In some cases, the epigenetically modified base of the nucleic acid sequence may comprise a methylated base, a hydroxymethylated base, a formylated base, or a carboxylic acid containing base or a salt thereof. In some cases, at least a portion of the nucleic acid sequence or the labeled nucleic acid sequence may be double-stranded. In some cases, the label may be associated with the epigenetically modified base by a single bond, a double bond, or a triple bond.

In some cases, the method may comprise separating the substantially complementary strand from the labeled nucleic acid sequence. In some cases, the nucleic acid sequence may comprise at least: from about 1 to about 3; from about 1 to about 5; from about 1 to about 10; from about 1 to about 15; or from about 1 to about 20 epigenetically modified bases per at least about 20 bases of the nucleic acid sequence. In some cases, the nucleic acid sequence may comprise at least about: 1, 5, 10, 15 or 20 epigenetically modified bases per at least about 20 bases of the nucleic acid sequence. In some cases, at least about: 70%, 75%, 80%, 85%, 90%, or 95% of bases of the substantially complementary strand may base pair with the labeled nucleic acid sequence. In some cases, the substantially complementary strand may hybridize to the nucleic acid sequence under stringent hybridization conditions.

In some cases, the nucleic acid sequence may comprise a cytosine guanine (CG) site, a cytosine phosphate guanine (CpG) island, or a combination thereof. In some cases, the nucleic acid sequence may comprise cell-free DNA. In some cases, the nucleic acid sequence may comprise a cDNA sequence. In some cases, the method may comprise sequencing an amplified product.

In some cases, the nucleic acid sequence may be from a sample. In some cases, the sample may be from a subject. In some cases, the subject may be a human. In some cases, the sample may comprise a buccal sample, a saliva sample, a blood sample, a plasma sample, a reproductive sample, a mucus sample, cerebral spinal fluid sample, a tissue sample, or any combination thereof.

In some cases, the method may comprise obtaining a result. In some cases, the method may comprise comparing the result to a reference. In some cases, the method may comprise communicating the result via a communication medium.

In some cases, the subject may be diagnosed with a condition. In some cases, the method may comprise diagnosing the subject as having a condition. In some cases, the method may comprise diagnosing the subject as having a likelihood of developing a condition. In some cases, the diagnosing may be based on the comparing the result to the reference. In some cases, the diagnosing may at least partially confirm a previous diagnosis. In some cases, the condition may be a cancer.

In some cases, the method may comprise selecting a treatment for the subject. In some cases, the method may comprise treating the subject. In some cases, the treating may comprise: surgery, chemotherapy, radiation therapy, immunotherapy, targeted therapy, hormone therapy, stem cell transplant, and precision medicine. In some cases, the method may comprise repeating the associating, the hybridizing and the amplifying at different time points.

In some cases, the subject may be a human. In some cases, the label may comprise a sugar. In some cases, the sugar may comprise a glucose. In some cases, the glucose may be modified.

In some cases, the label may be associated with the epigenetically modified base with the assistance of an enzyme. In some cases, the enzyme may be selective for a portion of the nucleic acid sequence that is double-stranded. In some cases, the label may be selectively associated with the epigenetically modified base at a portion of the nucleic acid sequence that is double-stranded. In some cases, the label may be selective for a portion of the nucleic acid sequence. In some cases, the portion may be double-stranded.

In some cases, the substantially complementary strand may be substantially free of an epigenetically modified base. In some cases, a substantially complementary strand may be free of an epigenetically modified base. In some cases, the amplifying may result in a plurality of nucleic acid strands. In some cases, less than about 2% of the plurality of nucleic acid strands may comprise an epigenetically modified base. In some cases, the nucleic acid sequence may comprise a plurality of epigenetically modified bases. In some cases, the substantially complementary strand may comprise less than about 2% of the plurality of epigenetically modified bases. In some case, the substantially complementary strand may comprise an epigenetically modified base.

Another aspect of the present disclosure provides a kit. In some cases, the kit may comprise: instructions for use; a container; a label configured to (i) associate with an epigenetically modified nucleic acid sequence and to (ii) associate with a substrate; a control nucleic acid sequence associated with a substrate and a substrate configured to associate with the label.

Another aspect of the present disclosure provides a method. In some cases, the method may comprise (a) associating a label with an epigenetically modified base of a nucleic acid sequence to form a labeled nucleic acid sequence; (b) hybridizing a substantially complementary strand to the labeled nucleic acid sequence; and (c) amplifying the substantially complementary strand in a reaction in which the labeled nucleic acid sequence is substantially not present.

Another aspect of the present disclosure provides a method. In some cases, the method may comprise (a) hybridizing a substantially complementary strand to a nucleic acid sequence comprising an epigenetically modified base; (b) associating a label with the epigenetically modified base of a nucleic acid sequence to form a labeled nucleic acid sequence; and (c) amplifying the substantially complementary strand in a reaction in which the labeled nucleic acid sequence is substantially not present.

In some cases, the label may be associated with a substrate. In some cases, the substrate may comprise a bead. In some cases, the bead may be a magnetic bead. In some cases, the substrate may comprise an array.

In some cases, the substantially complimentary strand may be shorter in length than the labeled nucleic acid sequence. In some cases, the substantially complimentary strand may be elongated before the amplifying. In some cases, the hybridizing may comprise hybridizing at least two substantially complementary strands to the labeled nucleic acid sequence. In some cases, the method may comprise ligating the at least two substantially complementary strands. In some cases, the labeled nucleic acid sequence may comprise an adapter sequence. In some cases, the hybridizing may comprise hybridizing at least a portion of the substantially complimentary strand to the adapter sequence.

In some cases, the nucleic acid sequence may comprise a first barcode. In some cases, the nucleic acid sequence may comprise a second barcode. In some cases, the first barcode may be a unique barcode and the second barcode is a sample barcode.

In some cases, the epigenetically modified base of the nucleic acid sequence may be a hydroxymethylated base (hmB). In some cases, the hmB may be 5-hydroxymethylated base (5-hmB). In some cases, the 5-hmB may be a 5-hydroxymethylated cytosine (5-hmC). In some cases, the epigenetically modified base of the nucleic acid sequence may comprise a methylated base, a hydroxymethylated base, a formylated base, or a carboxylic acid containing base or a salt thereof. In some cases, at least a portion of the nucleic acid sequence or the labeled nucleic acid sequence may be double-stranded. In some cases, the label may be associated with the epigenetically modified base by a single bond, a double bond, or a triple bond. In some cases, the method may comprise separating the substantially complementary strand from the labeled nucleic acid sequence.

In some cases, the nucleic acid sequence may comprise at least: from about 1 to about 3; from about 1 to about 5; from about 1 to about 10; from about 1 to about 15; or from about 1 to about 20 epigenetically modified bases per at least about 20 bases of the nucleic acid sequence. In some cases, the nucleic acid sequence may comprise at least about: 1, 5, 10, 15 or 20 epigenetically modified bases per at least about 20 bases of the nucleic acid sequence. In some cases, at least about: 70%, 75%, 80%, 85%, 90%, or 95% of bases of the substantially complementary strand may base pair with the labeled nucleic acid sequence. In some cases, the substantially complementary strand may hybridize to the nucleic acid sequence under stringent hybridization conditions.

In some cases, the nucleic acid sequence may comprise a cytosine guanine (CG) site, a cytosine phosphate guanine (CpG) island, or a combination thereof. In some cases, the nucleic acid sequence may comprise cell-free DNA. In some cases, the nucleic acid sequence may comprise a cDNA sequence. In some cases, the method may comprise sequencing an amplified product.

In some cases, the nucleic acid sequence may be from a sample. In some cases, the sample may be from a subject. In some cases, the subject may be a human. In some cases, the sample may comprise a buccal sample, a saliva sample, a blood sample, a plasma sample, a reproductive sample, a mucus sample, cerebral spinal fluid sample, a tissue sample, or any combination thereof.

In some cases, the method may comprise obtaining a result. In some cases, the method may comprise comparing the result to a reference. In some cases, the method may comprise communicating the result via a communication medium.

In some cases, the subject may be diagnosed with a condition. In some cases, the method may comprise diagnosing the subject as having a condition. In some cases, the method may comprise comprising diagnosing the subject as having a likelihood of developing a condition. In some cases, the diagnosing may be based on the comparing the result to the reference. In some cases, the diagnosing at least partially may confirm a previous diagnosis. In some cases, the condition may be a cancer.

In some cases, the method may comprise selecting a treatment for the subject. In some cases, the method may comprise treating the subject. In some cases, the treating may comprise: surgery, chemotherapy, radiation therapy, immunotherapy, targeted therapy, hormone therapy, stem cell transplant, and precision medicine. In some cases, the method may comprise repeating the associating, the hybridizing and the amplifying at different time points. In some cases, the subject may be a human.

In some cases, the label may comprise a sugar. In some cases, the sugar may comprise a glucose. In some cases, the glucose may be modified. In some cases, the label may be associated with the epigenetically modified base with the assistance of an enzyme. In some cases, the enzyme can be selective for a portion of the nucleic acid sequence that is double-stranded. In some cases, the label may be selectively associated with the epigenetically modified base at a portion of the nucleic acid sequence that is double-stranded. In some cases, the label may be selective for a portion of the nucleic acid sequence. In some cases, the portion may be double-stranded.

Another aspect of the present disclosure provides a kit. In some cases, the kit may comprise: instructions for use; a container; a label configured to (i) associate with an epigenetically modified nucleic acid sequence and to (ii) associate with a substrate; a control nucleic acid sequence associated with a substrate and a substrate configured to associate with the label.

In some cases, the substantially complementary strand may be substantially free of an epigenetically modified base. In some cases, the substantially complementary strand may be free of an epigenetically modified base. In some cases, the amplifying may result in a plurality of nucleic acid strands, wherein less than about 2% of the plurality of nucleic acid strands may comprise an epigenetically modified base. In some cases, the nucleic acid sequence may comprise a plurality of epigenetically modified bases, and wherein the substantially complementary strand may comprise less than about 2% of the plurality of epigenetically modified bases. In some cases, the substantially complementary strand may comprise an epigenetically modified base.

Another aspect of the present disclosure provides a method. In some cases, the method may comprise: detecting a presence of a plurality of epigenetically modified residues in a nucleic acid sequence, wherein the plurality of epigenetically modified residues comprises at least 2 epigenetically modified residues, and wherein a sensitivity of detection remains substantially constant with an increasing number of epigenetically modified residues in the plurality of epigenetically modified residues.

In some cases, the at least 2 epigenetically modified residues may be at least 4 epigenetically modified residues. In some cases, the sensitivity of detection may comprise detecting a presence of at least about 90% of the plurality of epigenetically modified residues. In some cases, the sensitivity of detection may comprise detecting a presence of each epigenetically modified residue of the plurality of epigenetically modified residues.

Another aspect of the present disclosure provides a method. In some cases, the method may comprise: enriching a nucleic acid sequence, wherein the nucleic acid sequence comprises (i) a plurality of epigenetically modified residues and (ii) a sequence length, wherein the plurality of epigenetically modified residues comprises at least 2 epigenetically modified residues, wherein the enriching comprises at least 4 cycles of amplification and produces a plurality of sequence reads, and wherein about 90% of the plurality of sequence reads retain at least about 90% of the sequence length.

In some cases, the at least 2 epigenetically modified residues may be at least 4 epigenetically modified residues. In some cases, the at least 4 cycles of amplification may be at least 8 cycles of amplification.

In some cases, the nucleic acid sequence may comprise cell-free DNA. In some cases, the nucleic acid sequence may comprise a cDNA sequence.

In some cases, an epigenetically modified residue of the plurality of epigenetically modified residues may be a hydroxymethylated base (hmB). In some cases, the hmB may be 5-hydromethylated base (5-hmB). In some cases, the 5-hmB may be a 5-hydroxymethylated cytosine (5-hmC). In some cases, an epigenetically modified residue of the plurality of epigenetically modified residues may comprise a methylated base, a hydroxymethylated base, a formylated base, or a carboxylic acid containing base or a salt thereof.

In some cases, at least a portion of the nucleic acid sequence may be double-stranded. In some cases, the nucleic acid sequence may comprise a cytosine guanine (CG) site, a cytosine phosphate guanine (CpG) island, or a combination thereof.

Another aspect of the present disclosure provides a method. In some cases, the method may comprise: enriching a nucleic acid sequence comprising a plurality of epigenetically modified residues to produce a plurality of sequence reads, wherein at least about 90% of the plurality of sequencing reads produced from the enriching are from about 1% to about 50% of a genome.

In some cases, the at least about 90% of the plurality of sequencing reads produced may be from about 1% to about 20% of the genome. In some cases, a length of the plurality of sequencing reads may be at least about 10 basepairs. In some cases, the plurality of epigenetically modified residues may be at least about 2 epigenetically modified residues. In some cases, the plurality of epigenetically modified residues may be at least about 6 epigenetically modified residues.

In some cases, a label may be associated with an epigenetically modified residue of the plurality of epigenetically modified residues. In some cases, the label may be associated with the epigenetically modified residue by a single bond, a double bond, or a triple bond.

In some cases, the nucleic acid sequence may comprise at least: from about 1 to about 3; from about 1 to about 5; from about 1 to about 10; from about 1 to about 15; or from about 1 to about 20 epigenetically modified residues per at least about 20 bases of the nucleic acid sequence. In some cases, the nucleic acid sequence may comprise at least about: 1, 5, 10, 15 or 20 epigenetically modified residues per at least about 20 bases of the nucleic acid sequence.

In some cases, the nucleic acid sequence may comprise cell-free DNA. In some cases, the nucleic acid sequence may comprise a cDNA sequence. In some cases, the nucleic acid sequence may be from a sample. In some cases, the sample may be obtained from a subject. In some cases, the subject may be a human. In some cases, the sample may comprise a buccal sample, a saliva sample, a blood sample, a plasma sample, a reproductive sample, a mucus sample, cerebral spinal fluid sample, a tissue sample, or any combination thereof.

In some cases, the method may further comprise obtaining a result. In some cases, the method may further comprise comparing the result to a reference. In some cases, the method may further comprise communicating the result via a communication medium.

In some cases, the subject may be diagnosed with a condition. In some cases, the method may further comprise diagnosing the subject as having a condition. In some cases, the method may further comprise diagnosing the subject as having a likelihood of developing a condition. In some cases, the diagnosing may be based on the comparing the result to the reference. In some cases, the diagnosing at least partially may confirm a previous diagnosis. In some cases, the condition may be a cancer.

In some cases, the method may further comprise selecting a treatment for the subject. In some cases, the method may further comprise treating the subject. In some cases, the treating may comprise: surgery, chemotherapy, radiation therapy, immunotherapy, targeted therapy, hormone therapy, stem cell transplant, and precision medicine.

In some cases, the label may comprise a sugar. In some cases, the sugar may comprise a glucose. In some cases, the glucose may be modified. In some cases, the label may be associated with the epigenetically modified residue with assistance of an enzyme. In some cases, the enzyme may be selective for a portion of the nucleic acid sequence that is double-stranded. In some cases, the label may be selectively associated with the epigenetically modified residue at a portion of the nucleic acid sequence that is double-stranded. In some cases, the label may be selective for a portion of the nucleic acid sequence. In some cases, the portion may be double-stranded.

Another aspect of the present disclosure provides a method. In some cases, the method may comprise: assaying the cell-free sample by next generation sequencing to identify a nucleic acid sequence, wherein a presence of a 5-hydroxymethylcytosine (5-hmC) in the nucleic acid sequence identifies the cell-free sample as malignant for the cancer. In some cases, the cell-free sample may be obtained from a subject having or suspected of having said cancer. In some cases, the method may further comprise selecting a treatment for the subject based on the presence of the 5-hmC. In some cases, the presence of the 5-hmC may comprise a level of 5-hmC in the cell-free sample. In some cases, the nucleic acid sequence may comprise a cytosine guanine (CG) site, a cytosine phosphate guanine (CpG) island, or a combination thereof. In some cases, the method may further comprise obtaining a result based on the presence of the 5-hmC. In some cases, the method may further comprise communicating the result via a communication medium. In some cases, a label may be associated with an epigenetically modified base of the nucleic acid sequence.

### EXAMPLE 1: A DIAGNOSTIC METHOD

A subject may be suspected of having a cancer. A sample comprising a nucleic acid sequence may be obtained from the subject by at least one of: a plasma sample, a serum sample, a blood sample, a urine sample, a buccal sample. The nucleic acid sequence may be isolated from the sample. Epigenetic modifications present on the nucleic acid sequence may be labeled with UDP-6-N₃-Glu employing T4 Phage beta-glucosyltransferase (T4-BGT) and/or with click chemistry. A substantially complementary strand may be hybridized to a. portion of the nucleic acid sequence comprising the epigenetic modifications. The nucleic acid sequence may be contacted with a substrate such that the labelled nucleic acid sequence comprising the epigenetic modifications may be bound to the substrate. The substrate may be washed with a washing buffer. The substantially complementary strand may be separated from the nucleic acid sequence that may be associated with the substrate. The substantially complementary strand may he amplified in the absence of the nucleic acid sequence. The subject may he diagnosed as having the cancer when a presence of an epigenetic modification associated with the cancer may be confirmed present in the sample obtained from the subject.

### EXAMPLE 2: hmC LABELLING

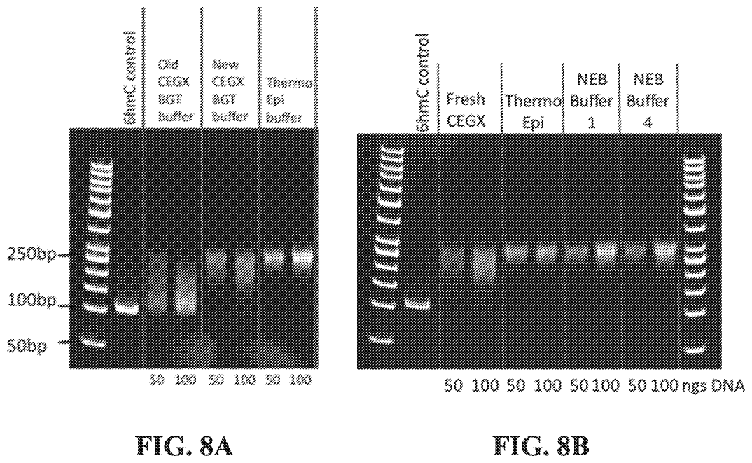

FIG. 8A-8B show a band shift assay of 6 5-hmC using different T4 Phage beta-glucosyltransferase (T4-BGT) buffers. Buffer A may comprise 25 mM MgCl₂, 50 mM Hepes, pH 8 stored at room temperature. Buffer A may be approximately 8 weeks old (‘old’ Buffer A) or freshly made (“fresh” Buffer A). Commercially available Thermo BGT buffer or “Epi” buffer may also be tested. NEB buffers 1 and 4 may also be tested. Samples of 50 nanograms (ng) or 100 ng DNA may be labelled for 6 5-hmC residues. As shown in FIG. 8A-B, the efficiency of labelling an epigenetic modified base (such as a 5-hmC base) in a template (such as a synthetic template) may vary depending on the reaction buffer used. In FIG. 8A-8B, using a 100-mer synthetic template containing 6 5-hmC residues, ‘old’ Buffer A may label with least efficiently (least pronounced bandshift with the most pronounced smear) as compared to the other buffers that label with higher efficiency and discrete bandshifts. “Fresh” Buffer A may provide better labeling, but may not go to completion (such as with the 100 ng DNA). Labelling with Thermo “Epi” buffer or NEB buffer 1 or buffer 4 may go to completion with the 50 ng and the 100 ng DNA sample.

### EXAMPLE 3: SEQUENCING METRICS

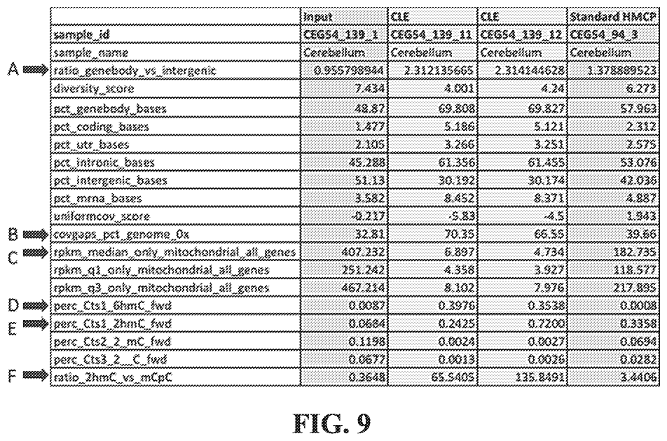

FIG. 9 shows a comparison of detailed sequencing metrics between 5-hmC pulldown (HMCP) and Copy Label Enrich (CLE) methods. “A” of FIG. 9 shows the CLE method may provide an increased ratio of reads in gene bodies vs intergenic regions, data that may be consistent with a more specific pulldown. “B” of FIG. 9 shows CLE may provide more gaps in coverage, data that may highlight fewer off-target reads and may be indicative of a cleaner background. “C” of FIG. 9 shows the CLE method may have very few mitochondrial reads (for example, mt DNA that may not be methylated or hydroxymethylated) and therefore this may be a proxy for off-target effects. The CLE method may be superior to the HMCP method by this measure. “D” of FIG. 9 shows CLE may have approximately equal numbers of reads from the control templates containing 2 and 6 5-hmC residues, respectively. In the HMCP method, there may be a differential of several orders of magnitude, for example, the template containing 6 5-hmC residues may be barely detected. “E” of FIG. 9 shows that a template containing 2 5-hmC residues may be detected at similar levels in both CLE and HMCP methods. “F” of FIG. 9 shows a ratio of spike control recovery (hmC:C/mC) may be much higher in the CLE method, indicating improved overall specificity.

### EXAMPLE 4: INTEGRATIVE GENOMICS VIEWER (IGV) SSCREENSHOTS

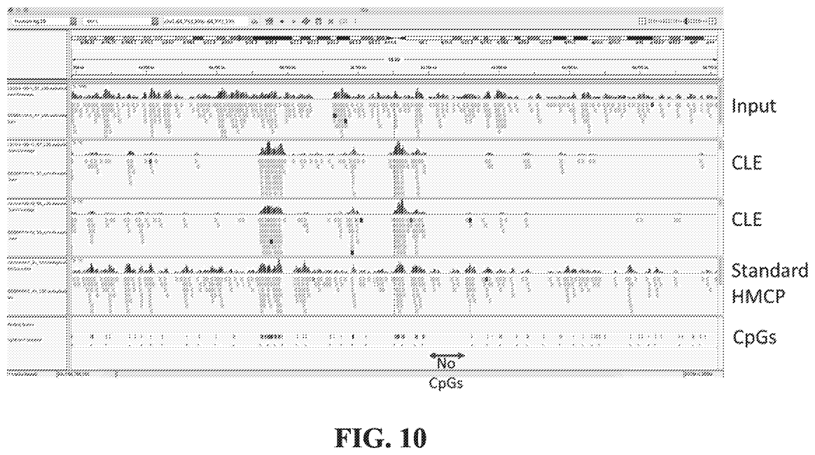

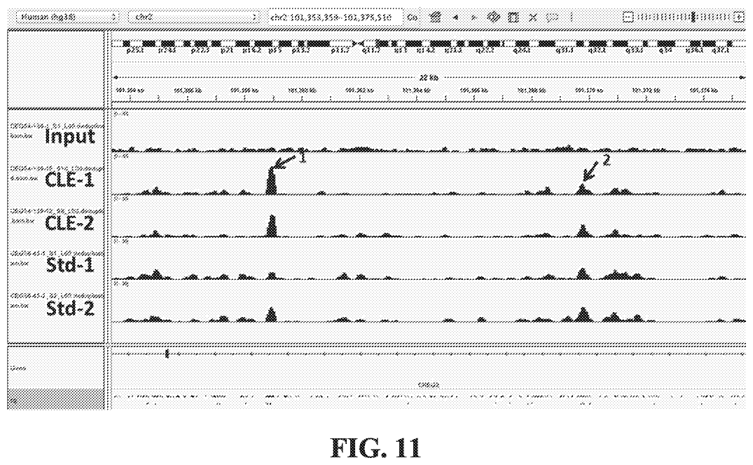

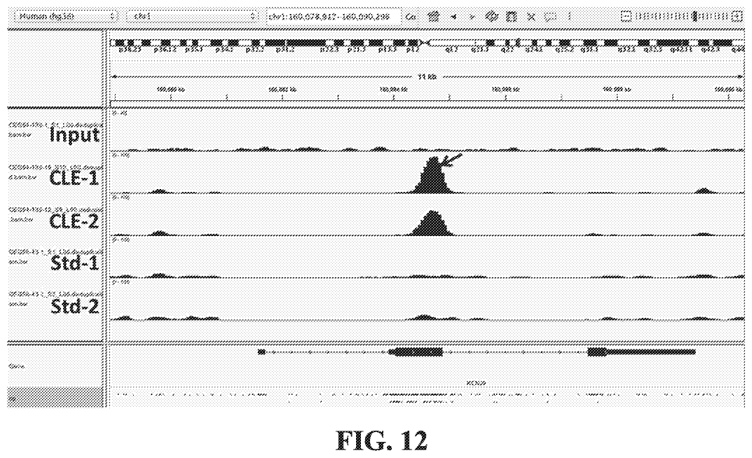

FIG. 10 shows an Integrative Genomics Viewer (IGV) screenshot of an 18 kb region of the human genome and the alignment of pulled-down reads. The reads from the CLE method may show larger, more discrete peaks and fewer reads in a region of ˜1 kilobase (kb) lacking any CpGs (annotated ‘No CpGs’ on FIG. 10). The data from the IGV screenshot of FIG. 10 may suggest cleaner background with the CLE method compared to the HMCP method. FIG. 11 shows another IGV screenshot of a different genomic region and may highlight the ability of the CLE method to pull down regions of brain whole genome DNA (wgDNA) with dense CpGs more effectively than the HMCP method. The first peak in FIG. 11 (annotated ‘1’ in the figure) is 19 CpGs in a 400 basepair (bp) region and may show enhanced enrichment in the CLE method as compared to the HMCP method. The second peak in FIG. 11 (annotated ‘2’ in the figure) is 9 CpGs in a 400 bp region and may show similar enrichment for both CLE method and the HMCP method. FIG. 12 shows a further IGV screenshot of a region of the human genome with dense CpGs and may show the ability of the CLE method to detect a strong peak that may not be detected at all by the HMCP method. For example, in a potassium voltage-gated channel subfamily J member 9 (KCNJ9)-enriched expression in a brain tissue sample and approximately 36 CpGs in a 500 basepair (bp) region, superior enrichment may be shown in the CLE method compared to the HMCP method, as shown by the annotated arrow in FIG. 12. This lack of detection from the HMCP method may be due to closely spaced 5-hmC residues inhibiting the processivity of the polymerase in a polymerase reaction, such as a library enrichment by PCR.

### EXAMPLE 5: SUMMARY METRICS COMPARISON

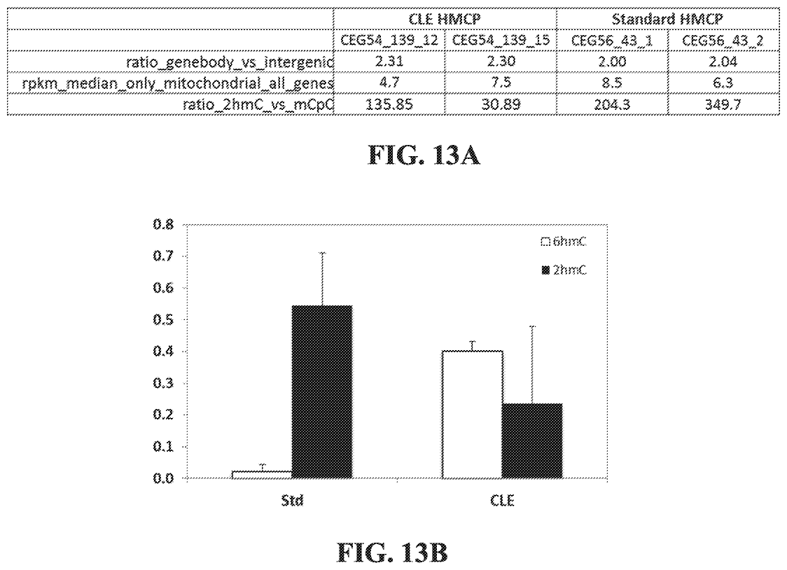

FIG. 13A-13B show summary metrics from the CLE method in comparison to the HMCP method comparing a) a ratio of gene bodies vs. intergenic regions, b) mitochondrial reads, c) a ratio of 2 5-hmC to mCpG in FIG. 13A and a graphical representation of the relative recovery of the 2 and 6 5-hmC control templates across the two different protocols as shown in FIG. 13B. An approximate 2-fold superior enrichment for 2 5-hmC may be observed when employing the HMCP method, FIG. 13B. A significantly increased enrichment of approximately 18 fold for 6 5-hmC may be observed when employing the CLE method, FIG. 13B.

### EXAMPLE 6: HMCP METHOD

### DNA Sample Preparation

Cell free DNA (cfDNA) may be extracted from plasma following Bioo Scientific's NextPrep-Mag cfDNA Isolation Kit instructions. When using formalin-fixed, paraffin-embedded (FFPE) genomic DNA (gDNA) extraction an appropriate kit may be chosen and a kit protocol followed. Genomic DNA (gDNA) from tissue may be fragmented. An appropriate amount of gDNA (100 ng ˜2000 ng) may be diluted in low-TE buffer and may be sheared to 150 basepair (bp) with Covaris in micro TUBE-50. DNA may be quantified by Qubit and QC by Bioanalyser to check size distribution. Controls may be spiked-in at 0.1% w/w (10 pg of each control for 10 nanogram (ng) fragmented gDNA or cfDNA)

### Pre-Pulldown DNA Library Preparation

Follow KAPA Hyper Prep Kit.

### End Repair and A-Tailing

Each end repair and A-tailing reaction may be assembled in a tube.

| Component | Volume | |
|---|---|---|
| Double-stranded DNA (cfDNA or fragmented gDNA) | 50 | μL |
| End Repair & A-Tailing Buffer* | 7 | μL |
| End Repair & A-Tailing Enzyme Mix* | 3 | μL |
| Total volume: | 60 | μL |

*The buffer and enzyme mix may be pre-mixed and added in a single pipetting.

The tube may be vortexed, may be spun down, and may be returned to ice. The tube may be incubated in a thermocycler programmed as outlined below:

| | | |
|---|---|---|
| Step | Temp | Time |

| | | | |
|---|---|---|---|
| End repair and A-tailing | 20° | C. | 30 min |
| | 65° | C.* | 30 min |
| Hold | 4° | C. | ∞ |

*Set temperature of heated lid to 85° C.

### Adapter Ligation

DNA barcoded adapters from Bioo Scientific (NEXTflex DNA Barcodes-24) may be used for ligation. Adaptors may be diluted according to the amount of DNA used: 0.3 μM for 1 ng; 3 μM for 10 ng; 15 μM for 100 ng. In a same tube(s) in which end repair and A-tailing may be performed, each adapter ligation reaction may be assembled as follows:

| Component | Volume | |
|---|---|---|
| End repair and A-tailing reaction product (from | 60 | μL |
| [00163]) | | |
| Adapter stock (concentration as required) | 5 | μL |
| PCR-grade water* | 5 | μL |
| Ligation Buffer* | 30 | μL |
| DNA Ligase* | 10 | μL |
| Total volume: | 110 | μL |

*The water, buffer and ligase enzyme may be premixed and added in a single pipetting.

The tube may be mixed thoroughly and may be centrifuged briefly. The tube may be incubated at 20° C. for 45 minutes (min).

### Post-Ligation Cleanup

In the same tube(s), a 0.8X bead-based cleanup may be performed by combining the following:

| Component | Volume | |
|---|---|---|
| Adapter ligation reaction product | 110 | μL |
| AMPure Beads | 88 | μL |
| Total volume: | 198 | μL |

The tube(s) may be mixed thoroughly by vortexing and may be incubated at room temperature for 10 minutes so that DNA may bind to the beads. The tube(s) may be placed on a magnet (such that the beads may be captured) until the liquid may be clear. The supernatant may be removed and discarded. The residual fluid at the bottom of the tube may be collected by popping the spin tube and then returning the tube to magnet for a few seconds, removing and discarding the residual fluid. The tube(s) may be kept on the magnet and 200 μL of 80% ethanol may be added. The tube(s) may be incubated on the magnet at room temperature for ˜30 seconds. The ethanol may be removed and discarded. The tube(s) may be kept on the magnet and 200 μL of 80% ethanol may be added. The tube(s) may be incubated on the magnet at room temperature for approximately 30 seconds. The ethanol may be removed and discarded. The beads may be dried at room temperature for 3-5 minutes (or until all the ethanol has evaporated). The AMPure beads may be thoroughly resuspended in 20 μL of H₂O. The tube(s) may be incubated at room temperature for 5 minutes to elute DNA off the beads. The tube(s) may be placed on a magnet to capture the beads and may be incubated until the liquid may be clear. The clear supernatant may be transferred to a new tube(s). One μL may be kept for Input Library Amplification. 18 μL may be transferred to fresh tube(s) and may continue to the 5-hmC labelling reaction and click chemistry.

### Input Library Amplification and Purification

### Amplification

One μl of pre pull-down library disclosed herein may be mixed with 9 μl of 10 mM Tris-HCl (pH8.0), 2 μl of this 10 times diluted library may be used for amplification in 50 μL using KAPA HiFi Hotstart DNA Polymerase.

Assemble each library amplification reaction may be as follows:

| Component | Volume | |
|---|---|---|
| Diluted Pre Pull-down Library | 2 | μL |
| H₂O | 29.5 | μL |
| 5× KAPA HiFi Buffer | 10 | μL |
| 10 mM dNTPs | 1.5 | μL |
| 10 uM ILM UNI Oligo | 3 | μL |
| 10 uM ILM IDX Oligo | 3 | μL |
| KAPA HiFi HotStart Polymerase | 1 | μL |
| Total volume | 50 | μL |

A tube may be mixed thoroughly and may be centrifuged briefly. The following cycling protocol may be utilized for amplification:

| Step | Temp | Time | | Cycles |
|---|---|---|---|---|
| Initial Denaturation | 95° C. | 3 | min | 1 |
| Denaturation | 98° C. | 20 | s | 16 cycles |
| Annealing | 55° C. | 30 | s | |
| Extension | 72° C. | 60 | s | |
| Final extension | 72° C. | 5 | min | 1 |

| | | | |
|---|---|---|---|
| Hold | 4° C. | ∞ | 1 |

### Post Amplification Cleanup

50 μL AMPure beads may be added to the 50 μL of PCR reaction from paragraph [00259], and may be vortexed to mix, and may be incubate at room temperature for 10 minutes (min). The tube(s) may be placed on a magnet to capture the beads until the liquid may be clear. The supernatant may be removed and discarded.

The tube(s) may be kept on the magnet and 200 μL of 80% ethanol may be added. The tube(s) may be incubated on the magnet at room temperature for approximately 30 seconds. The ethanol may be removed and discarded. The tube(s) may be kept on the magnet and 200 μL of 80% ethanol may be added. The tube(s) may be incubated on the magnet at room temperature for approximately 30 seconds. The ethanol may be removed and discarded. The beads may be dried at room temperature for 3-5 minutes (or until all the ethanol has evaporated). The AMPure beads may be thoroughly resuspended in 10 μL of 10 mM Tris-HCl (pH 8.0). The tube(s) may be incubated at room temperature for 5 minutes to elute DNA off the beads. The tube(s) may be placed on a magnet to capture the beads and incubated until the liquid may be clear. The clear supernatant may be transferred to a new tube(s). The samples may be stored at −20° C. Qubit and Bioanalyser may be utilized to quantify the library.

5-hmC Labelling Reaction and Click Chemistry.

Each labelling reaction may be assembled in a tube as follows.

| Component | Volume | |
|---|---|---|
| Pre Pull-down library (from paragraph [00256]) | 18 | μL |
| Nuclease Free Water | 2.5 | μL |
| 10× β-GT Buffer | 2.5 | μL |
| 2.5 mM UDP-6-N₃-Glu | 1 | μL |
| 5 U/μl T4 β-GT | 1 | μL |
| Total volume: | 25 | μL |

A tube may be mixed thoroughly and centrifuged briefly and incubated at 37° C. for 30 minutes in a Thermocycler. One μL of 20 mM DBCO-PEG₄-Biotin may be added to each tube at the end of reaction. The tube may be mixed thoroughly and centrifuged briefly, and incubated for 2 hours (hrs) at 37° C. in a Thermocycler. One μL of 10 mg/ml Salmon Sperm DNA may be added to the reaction mixture.

### Pre-Pulldown Micro Bio-Spin P30 Column Cleanup

The Micro Bio-Spin P30 column may be inverted several times to resuspend the settled gel and remove any bubbles. The tip may be snapped off and placed the column in a 2.0 ml collection tube. The top cap may be removed. The excess packing buffer may be allowed to drain by gravity to the top of the gel bed (about two minutes). The drained buffer may be discarded and then the column may be placed back into the 2.0 ml tube. Centrifugation may occur for 2 minutes at 1,000 g to remove the remaining packing buffer. The buffer may be discarded. 500 μL of Bead Blocking Buffer 1 (BBB1) buffer may be applied to the column and may be centrifuged 2 minutes at 1000 g, and buffer from collection tube may be discarded. Another 500 μL of BBB1 buffer may be applied to the column, and the buffer may be allowed to drain by gravity for about 2 minutes, and the buffer may be discarded in collection tube, and then may centrifuged 2 minutes at 1000 g, and the buffer may be discarded from collection tube. The column may be placed in a new 1.5 ml DNA LowBind eppendorf tube. The reaction mixture from paragraph [00263] may be loaded to the centre of the column, and may be centrifuged for 4 minutes at 1000 g to collect DNA (˜40 μL).

### Affinity Pulldown

M-270 Streptavidin beads may be blocked with Salmon Sperm DNA: The beads in the vial may be vortexed to be resuspended and the required volume (x=20% extra of number of samples processed) of beads may be transferred to a 1.5 ml eppendorf tube. The beads may be washed with 500 μL BBB1 buffer and resuspended. The tube may be placed on a magnet for 1 minute and the supernatant may be discarded. Washing of beads may be repeated twice, for a total of 3 washes. The beads may be resuspended in 500 μl of 100 μg/m1 Salmon Sperm DNA in BBB1 buffer (add 5 μl of 10 mg/ml Salmon Sperm DNA to 495 μl BBB1 buffer) and may be incubated for 30 minutes at room temperature with mixing. Washing of beads may be repeated twice. Residual fluid at bottom of tube may be collected, then the tube may be returned to magnet for a few seconds. The residual buffer may be removed and discarded. Beads in the same volume of BBB1 buffer may be resuspended as the initial volume of magnetic beads taken from the vial. One μL blocked M-270 Streptavidin beads may be added to reaction mixture from paragraph [00264], and may be incubated at 1300 rpm at room temperature for 30 minutes in a Thermomixer. A tube may be placed on magnetic stand for approximately 1 minute, and the supernatant may be removed and discarded.

### Post-Pulldown Streptavidin Beads Washes

200 μL BBB1 may be added to tube(s) from paragraph [00265] and may be incubated at room temperature for 5 minutes at 1500 rotations per minute (rpm) in a Thermomixer. The tube may be placed on a magnetic stand for approximately 2 minutes to remove and discard supernatant. The wash may be repeated twice. 200 μL BBB2 may be added and the tube may be incubated at room temperature for 5 minutes at 1500 rpm in a Thermomixer. The tube may be placed on a magnetic stand for approximately 2 minutes to remove and discard supernatant. The wash may be repeated twice. 200 μL BBB3 may be added and the tube may be incubated at room temperature for 5 minutes at 1500 rpm in a Thermomixer. The tube may be placed on magnetic stand for approximately 2 minutes to remove and discard supernatant. The wash may be repeated twice. 200 μL BBB4 may be added and the tube may be incubated at 55° C. for 5 minutes at 1500 rpm in a Thermomixer. The tube may be placed on a magnetic stand for approximately 2 minutes to remove and discard supernatant. The wash may be repeated twice. A final wash using 50 μL of H₂O may be performed without disturbing the beads. Pop spin may be performed to remove residual buffer. The beads may be resuspended in 20 μL of H₂O, and the bead suspension may be transferred to 0.2 ml PCR strip tubes.

### HMCP Library Enrichment by PCR

Each library amplification reaction may be assembled as follows:

| Component | Volume | |
|---|---|---|
| Pull-down Library | 20 | μL |
| H₂O | 14.5 | μL |
| 5× KAPA HiFi Buffer | 10 | μL |
| 10M dNTPs | 1.5 | μL |
| 10 μM NEXTflex primer mix | 3 | μL |
| KAPA HiFi HotStart | 1 | μL |
| Polymerase | | |
| Total volume | 50 | μL |

A tube may be mixed thoroughly and centrifuged briefly and amplified using the following cycling protocol:

| | | | |
|---|---|---|---|
| Step | Temp | Time | Cycles |

| | | | | | |
|---|---|---|---|---|---|
| Initial Denaturation | 95° | C. | 3 | min | 1 |
| Denaturation | 98° | C. | 20 | s | 16 cycles |
| Annealing | 55° | C. | 30 | s | |
| Extension | 72° | C. | 60 | s | |
| Final extension | 72° | C. | 5 | min | 1 |

| | | | | |
|---|---|---|---|---|
| Hold | 4° | C. | ∞ | 1 |

The tube(s) may be placed on a magnet to capture the beads until the liquid may be clear. The supernatant may be transferred to fresh tube (s). To the 50 μL of PCR reaction, 50 μL of AMPure beads may be added, and may be vortexed to mix and may be incubated for 10 minutes at room temperature. The tube may be centrifuged briefly and beads may be precipitated using a magnetic rack for 5 minutes at room temperature. Supernatant may be carefully removed and may be washed twice with 200 μL of 80% ethanol without disturbing the beads. The beads may be left on the magnetic rack, with the lids open, until dry (5-10 minutes). 10 μL of 10 mM Tris-HCl (pH 8.0) may be added to the beads and may be incubated at room temperature for 5 minutes. The supernatant may be collected in a fresh tube. Qubit and Bioanalyser may be employed to quantify the library.

### Buffer Preparation

10× Glucosylation Buffer (10× β-GT Buffer) (1 mL):
500 μL 1M HEPES (pH 8.0)
250 μL 1M MgCl₂(1 M)
250 μL H₂O

BBB1: Bead Blocking Buffer 1 (100 mL)
500 μL 1 M Tris (pH 7.5)
100 μL 0.5 M EDTA
20 ml 5 M NaCl
200 μL Tween20
79.2 mL H₂O

BBB2: Bead Blocking Buffer 2 (100 ml):
500 μl 1M Tris (pH7.5)
100 μl 0.5M EDTA
200 μl Tween20
99.2 ml H₂O

BBB3: Bead Blocking Buffer 3 (100 ml):
500 μl 1M Tris (pH9)
100 μl 0.5M EDTA
20 ml 5M NaCl
200 μl Tween20
79.2 ml H₂O

BBB4: Bead Blocking Buffer 4 (100 ml):
500 μl 1M Tris (pH9)
100 μl 0.5M EDTA
200 μl Tween20
99.2 ml H₂O

### EXAMPLE 7: HMCP CLE METHOD (AS SCHEMATICALLY SHOWN IN ONE EMBODIMENT IN FIG. 3)

Shearing of Genomic DNA (gDNA) to 150 bp

Genomic DNA (gDNA) may be diluted with low-TE buffer and may be sheared to 150 basepair (bp) with a Covaris in a micro TUBE-50. In some cases, purified cell-free DNA (cfDNA) can be used without the shearing.

### Library Preparation

Library preparation may follow KAPA Hyper Prep kit protocol using DNA barcoded adapters (24-plex) from Bioo Scientific. The adaptors may be diluted based on the input DNA used: 0.3 μM for 1 ng; 3 μM for 10 ng; 15 μM for 100 ng. For the final purification of the DNA, elution may occur in 12 ul of water. In some cases, a spike-in control may be employed. In such cases, a spike in the controls may occur at 0.1% weight by weight (w/w) before library preparation (10 picogram (pg) of each control 5-C, 5-mC, and 5-hmC; 5-hmC control can contain either 2 or 6 5-hmC).

### Primer Extension

a. Anneal NEXTflex primer to adapted DNA.

| | | |
|---|---|---|
| DNA (from Library Prep) | 12 | μl |
| 10× NEB buffer 4 | 2 | μl |
| 10 mM dNTPS | 2 | μl |
| 10 uM NEXTflex primer | 2 | μl |
| | 18 | μl |

95° C. for 3 mins
−0.1° C. per second to 14° C.

b. Klenow extension
Add 2 μl Klenow exo- (5U/μl)
37° C. for 30 mins
75° C. for 20 mins
   5-hmC Labelling Reaction and Click Chemistry

a. Labelling of 5-hmC

| | | |
|---|---|---|
| Extended DNA(From b. above) | 20 | μl |
| Water | 2.5 | μl |
| 10× NEB buffer 4 | 0.5 | μl |
| 2.5 mM UDP-6-N₃-Glu | 1 | μl |
| T4 β-GT (5 U/μl) | 1 | μl |
| | 25 | μl |

Incubate at 37° C. for 30 min.

A negative control without UDP-6-N₃-Glu or βGT can be included.

b. Click reaction
Add 1 μl of 20 mM DBCO-PEG₄-Biotin into the reaction and incubate for 2 hrs at 37° C.
Add 1 μl of 10 mg/ml Salmon Sperm DNA at the end of the incubation.

### Post Labelling Purification and Affinity Pull-Down

Buffer exchange may occur in a Micro spin P30 column with 500 μl of bead blocking buffer 1 (BBB1) twice (spin for 2 minutes each time at 1000 g). The reaction mixture from b. above may be loaded to the centre of column, and may be centrifuged for 4 minutes (mins) at 1000 g to collect the DNA in a DNA low-bind eppendorf tube.

The M270 streptavidin beads may be prepared. A) 10 μl of M-270 may be washed in 500 μl BBB1 twice. B) The beads may be blocked in 500 μlof BBB1 containing 100 μg/ml Salmon Sperm DNA at room temperature for 30 minutes on a rotator. C) The beads may be washed twice in 500 μl of BBB1. D) The beads may be resuspended in 10 μl of BBB1. One μl of the blocked M-270 Streptavidin beads may be added to the purified DNA from paragraph [00277] and may be incubated in a Thermomixer at 22° C. for 30 minutes at 1300 rotations per minute (rpm).

### Bead Washes

The beads may be washed in 200 μl bead blocking buffer 1 (BBB1) in a Thermomixer at 1500 rpm for 5 minutes at 22° C. and the washing may be repeated for a total of 3 washes. Then, the beads may be washed in 200 μl BBB2 in a Thermomixer at 1500 rpm for 5 minutes at 22° C. and the washing may be repeated for a total of 3 washes. Next, the beads may be washed in 200 μl BBB3 in a Thermomixer at 1500 rpm for 5 minutes at 22° C. and the washing may be repeated for a total of 3 washes. Then, the beads may be washed in 200 μl BBB4 in a Thermomixer at 1500 rpm for 5 minutes at 55° C. and the washing may be repeated for a total of 3 washes. A final wash with 50 μl of H₂O may occur without mixing. The water wash may be removed. 20 μl 0.1N NaOH may be added to the beads, and the beads may be resuspended and incubated in a Thermomixer at 1300 rpm for 10 minutes at 22° C. Using a magnet, the NaOH supernatant may be removed from the beads and pipetted into a new tube. Immediately the NaOH supernatant may be neutralized with 10 μl 0.2 M Tris pH 7.0. Continue to library enrichment by PCR.

### Library Enrichment by PCR

a. Set up PCR

| | | |
|---|---|---|
| DNA (from after Bead Washes) | 30 | μl |
| 5× KAPA HiFi Buffer | 10 | μl |
| 10 mM dNTPs | 1.5 | μl |
| 10 uM NEXTflex primer mix | 4 | μl |
| KAPA HiFi | 1 | μl |
| H₂O | 3.5 | μl |
| | 50 | μl |

Input DNA (no enrichment) can also be amplified. The library may be diluted 10 fold and may use 2 ul in the PCR.

PCR Programme

| | | |
|---|---|---|
| 95° C. for 3 mins | | |
| 98° C. for 20 secs | | |
| 55° C. for 30 secs | {close oversize brace} | 12 cycles |
| 72° C. for 1 min | | |
| 72° C. for 5 mins | | |
| 4° C. storage | | |

b. The amplified products may be purified using 1X AMPure XP beads and the DNA may be eluted in 10 μl of 10 mM Tris-HCl pH 8.0.

### Library QC and Sequencing

Qubit and Bioanalyser analysis

### Buffers

BBB1: Bead Blocking Buffer 1 (100 ml):
500 μl 1 M Tris pH 7.5
100 μl 0.5M EDTA
20 ml 5 M NaCl
200 μl Tween20
79.2 ml H₂O

BBB2: Bead Blocking Buffer 2 (100ml):
500 μl 1 M Tris pH 7.5
100 μl 0.5 M EDTA
200 μl Tween20
99.2 ml H₂O

BBB3: Bead Blocking Buffer 3 (100 ml):
500 μl 1 M Tris pH 9
100 μl 0.5 M EDTA
20 ml 5M NaCl
200 μl Tween20
79.2 ml H₂O

BBB4: Bead Blocking Buffer 4 (100 ml):
500 μl 1 M Tris pH 9
100 μl 0.5 M EDTA
200 μl Tween20
99.2 ml H₂O

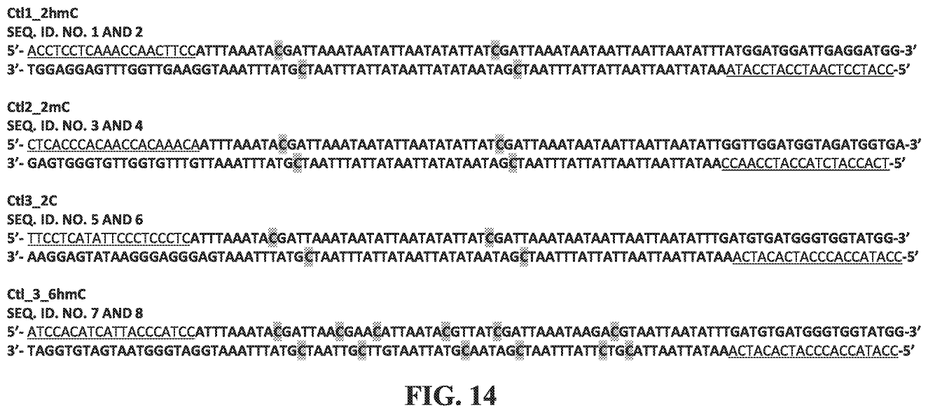

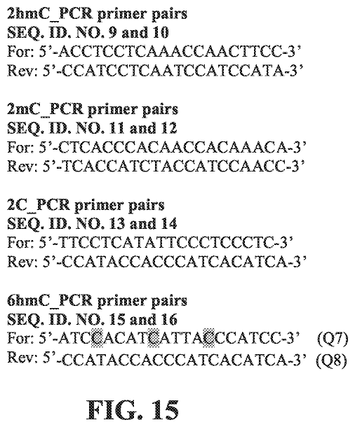

FIG. 14 and FIG. 15 show examples of different spike-in controls. A spike-in control may be employed in any method as described herein. For example, a spike-in control may be employed in a HMCP_CLE method, such as the spike-in controls shown in FIG. 14. In some cases, a spike-in control may be employed in a HMCP method, such as the spike-in controls shown FIG. 15. A spike-in control may be a sequence wherein a cytosine (C) residue may be replaced with a 5-methylated cytosine (5-mC) or a 5-hydroxymethylated cytosine (5-hmC) in a reaction, such as a PCR or sequencing reaction. Cytosine bases highlighted in grey in sequences of FIG. 14 may represent cytosine residues that may be replaced with a 5-mC or a 5-hmC. FIG. 15 also shows examples of PCR primer pairs for 2hmC, 2mC, 2C, and 6hmC.

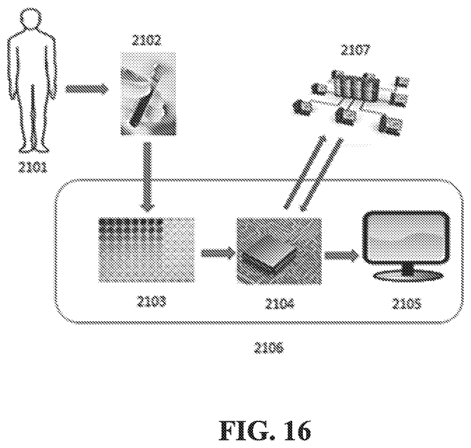

In some cases, as shown in FIG. 16, a sample 2102 may be obtained from a subject 2101, such as a human subject. A sample 2102 may be subjected to one or more methods as described herein, such as performing an assay. In some cases, an assay may comprise hybridization, amplification, sequencing, labeling, epigenetically modifying a base, or any combination thereof. One or more results from a method may be input into a processor 2104. One or more input parameters such as a sample identification, subject identification, sample type, a reference, or other information may be input into a processor 2104. One or more metrics from an assay may be input into a processor 2104 such that the processor may produce a result, such as a diagnosis or a recommendation for a treatment. A processor may send a result, an input parameter, a metric, a reference, or any combination thereof to a display 2105, such as a visual display or graphical user interface. A processor 2104 may (i) send a result, an input parameter, a metric, or any combination thereof to a server 2107, (ii) receive a result, an input parameter, a metric, or any combination thereof from a server 2107, (iii) or a combination thereof.

### EXAMPLE 8: COMPARISONS OF THE HMCP AND CLE METHODS USING VARIOUS OUTPUT METRICS

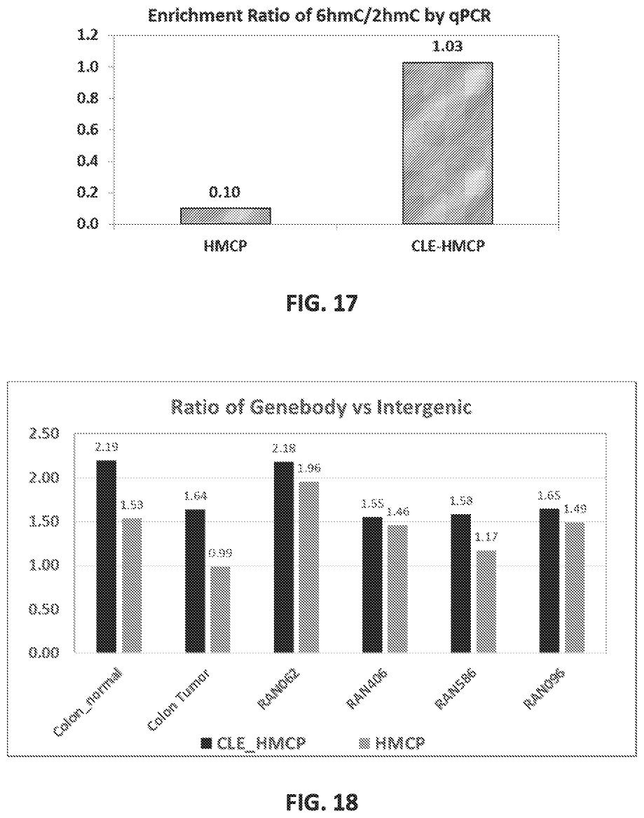

FIG. 17 shows a comparison of an enrichment ratio of 6 5-hmC/2 5-hmC by quantitative polymerase chain reaction (qPCR) between HMCP and CLE methods. The CLE method as compared to the HMCP method may advantageously relieve PCR bias against sequences having regions with dense 5-hydroxymethylated cytosines (5-hmC). For example, a sequence having at least about: 4, 5, 6, 7, 8, 9, 10 or more 5-hmC may be a dense region. In some cases, a sequence having at least about 4 5-hmC per 4, 6, 8, 10, 12, 16, 18, 20, 24, 48 bases or more may be a dense region. In FIG. 17, qPCR may be utilized to quantify a specific enrichment of 2 5-hmC and 6 5-hmC spike-in controls. FIG. 17 shows equal enrichment of 2 5-hmC and 6 5-hmC employing the CLE method. In some cases, the CLE method may provide at least about 80%, 85%, 90%, 95%, 99% or more enrichment of 6 5-hmC as compared to 2 5-hmC. In some cases, the CLE method may provide from about 80% to 100% enrichment of 6 5-hmC as compared to 2 5-hmC. In some cases, the CLE method may provide from about 90% to 100% enrichment of 6 5-hmC as compared to 2 5-hmC. In some cases, the CLE method may provide from about 95% to 100% enrichment of 6 5-hmC as compared to 2 5-hmC.

In contrast, FIG. 17 shows for the HMCP method only about 10% enrichment of 6 5-hmC as compared to 2 5-hmC. In some cases, the HMCP method may provide less than about 30%, 25%, 20%, 15%, 10%, 5%, 4%, 3%, 2% or less enrichment of 6 5-hmC as compared to 2 5-hmC. In some cases, the HMCP method may provide from about 1% to about 10% enrichment of 6 5-hmC as compared to 2 5-hmC. In some cases, the HMCP method may provide from about 1% to about 5% enrichment of 6 5-hmC as compared to 2 5-hmC. In some cases, the HMCP method may provide from about 1% to about 20% enrichment of 6 5-hmC as compared to 2 5-hmC.

FIG. 18 shows a comparison of a ratio of reads that map to inside genebodies compared to those that map to intergenic regions between HMCP and CLE methods. The ratio of amount of reads that map inside genebodies compared to those reads that map to intergenic regions may reflect the level of enrichment of 5-hmC in a given method. The CLE method outperforms the HMCP method in tissue genomic DNA (gDNA), for example, colon tumour tissue and normal colon tissue, as shown in FIG. 18. The CLE method also outperforms the HMCP method in four different cell free DNA (cfDNA) samples identified as RAN062, RNA406, RNA586, and RAN096 in FIG. 18.

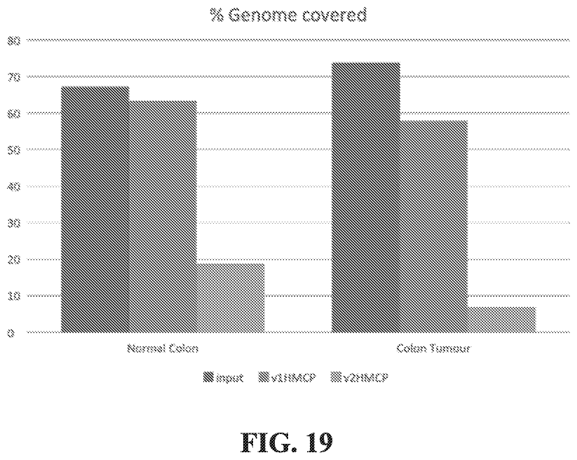

FIG. 19 shows a comparison of a percentage of the genome covered between HMCP and CLE methods. In some cases, 5-hmC may be a relatively rare modification. In some cases, large regions of a genome may be lacking in this modification. Therefore, a given method may leave significant areas of the genome uncovered when sequenced reads are mapped back to a reference assembly, such as hg38. For example, 10 nanogram (ng) sheared whole genomic DNA (wgDNA) from colon tumour tissue and normal colon tissue is analysed using HMCP and CLE methods. The percentage of the genome covered by mapped reads is shown in the histogram of FIG. 19. For example, from about 8% to about 20% of the genome may be covered when the CLE method is used compared with from about 55% to about 65% when the HMCP method is used, as shown in FIG. 19. The bar representing ‘input’ in FIG. 19 relates to how reads fall across the genome in the absence of any enrichment. In some cases, this may indicate that whilst the HMCP method may usually leave a detectable background coverage similar to input, this may be effectively reduced when the CLE method is used.

In some cases, less than about: 1%, 2%, 3%, 4%, 5%, 6%, 7%, 8%, 9%, 10%, 11%, 12%, 13%, 14%, 15%, 16%, 17%, 18%, 19%, 20%, 25%, or 30% of the genome may be covered when the CLE method is used. In some cases, from about 1% to about 30% of the genome may be covered when the CLE method is used. In some cases, from about 1% to about 20% of the genome may be covered when the CLE method is used. In some cases, from about 1% to about 15% of the genome may be covered when the CLE method is used. In some cases, from about 1% to about 10% of the genome may be covered when the CLE method is used. In some cases, from about 5% to about 25% of the genome may be covered when the CLE method is used.

In some cases, more than about: 50%, 55%, 60%, 65%, 70%, 75%, 80%, 85%, or 90% of the genome may be covered when the HMCP method is used. In some cases, from about 50% to about 85% of the genome may be covered when the HMCP method is used. In some cases, from about 50% to about 80% of the genome may be covered when the HMCP method is used. In some cases, from about 50% to about 70% of the genome may be covered when the HMCP method is used. In some cases, from about 60% to about 80% of the genome may be covered when the HMCP method is used. In some cases, from about 60% to about 90% of the genome may be covered when the HMCP method is used.

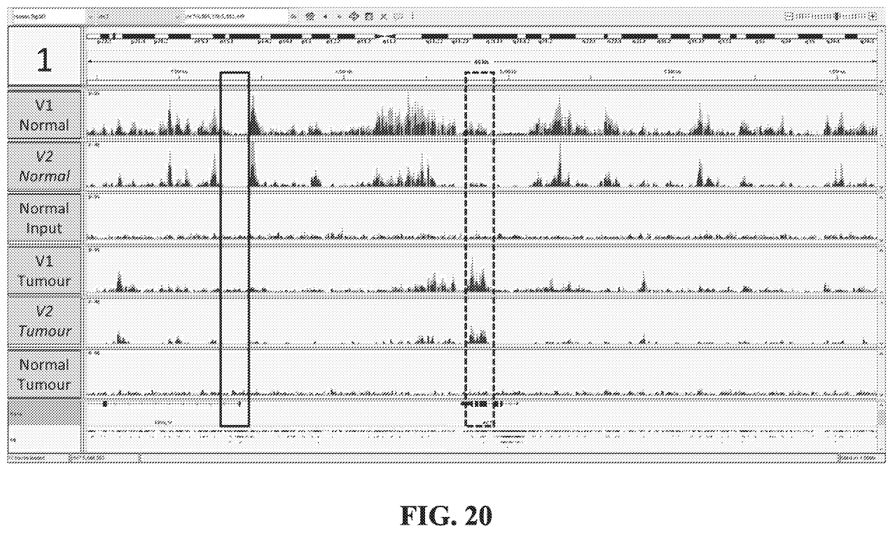

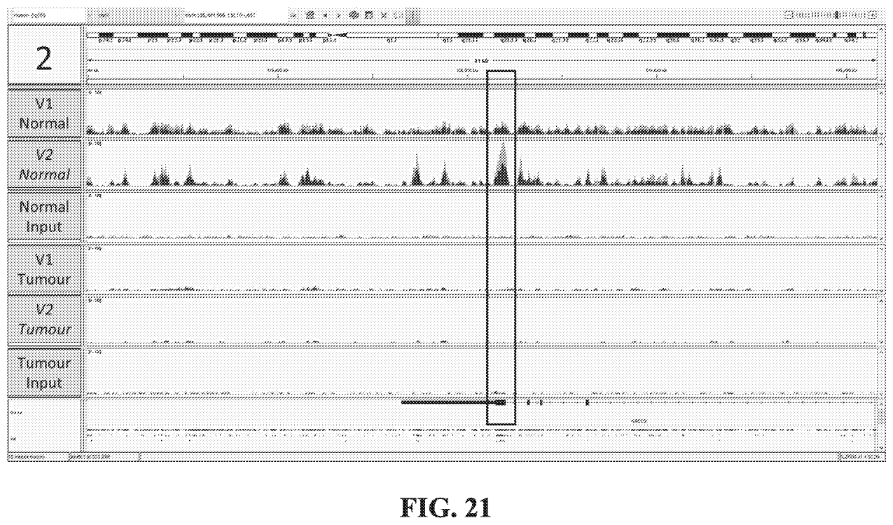

In FIG. 20 through FIG. 25, similar Integrative Genomics Viewer (IGV) screenshots are examined for examples that may highlight the effects of the different methods at specific loci. The data in FIG. 20 through FIG. 25 are obtained from IGV screenshots comparing HMCP and CLE methods on whole genomic DNA (wgDNA) extracted from normal colon tissue and colon tumour tissue. Also shown is the input sample for each DNA source which shows a distribution of reads in the absence of any enrichment/pulldown steps. In some cases, tumour cell DNA may have significantly less 5-hmC than corresponding normal tissue and this may be reflected in most views of the genome irrespective of method deployed. Unless otherwise stated, all of the data in FIG. 20 through FIG. 25 is Integrative Genomics Viewer (IGV) plots with a vertical scaling of 0-50 reads. The data is generated with 10 nanograms (ng) of the relevant input DNA sheared to a fragment size of 150 base pairs (bp) and processed according to HMCP or CLE method.

FIG. 20 shows an IGV screenshot comparing HMCP and CLE methods at the beta-actin (ACTB) locus. In this example, the 5-hmC peak (dotted line box) in the tumour DNA may be more pronounced than in the normal tissue, and this may be seen irrespective of the method used. In the solid line box, the lack of 5-hmC at the start of the adjacent FXBL18 gene may be much clearer in the CLE method than the HMCP method. The latter may have background reads at a similar level to input which may reduce a signal:noise ratio.

FIG. 21 shows an IGV screenshot comparing HMCP and CLE methods at the start of the NaCC2 locus. In FIG. 21, the vertical scale has been reset in each panel to 150 reads. In FIG. 21, the start of the NACC2 locus is shown. This gene encodes a transcriptional co-repressor that stabilises p53 via MDM2. The CLE method shows an extent of differential 5-hmC throughout the gene including a large peak at the first exon in healthy colon whole genomic DNA (wgDNA) (solid lined box). The HMCP method fails to reveal this level of granularity, instead demonstrating a more even distribution across the gene. The loss of 5-hmC in this gene in the tumour DNA may reflect loss of transcriptional control that may be contributing to, or a consequence of, malignant transformation. The CLE method highlights a difference between tumour and normal DNA in this particular genomic region in a way that the HMCP method does not. In some cases, the CLE method may be superior to the HMCP method and provide a higher level of granularity of distribution of one or more epigenetic modifications throughout a gene.

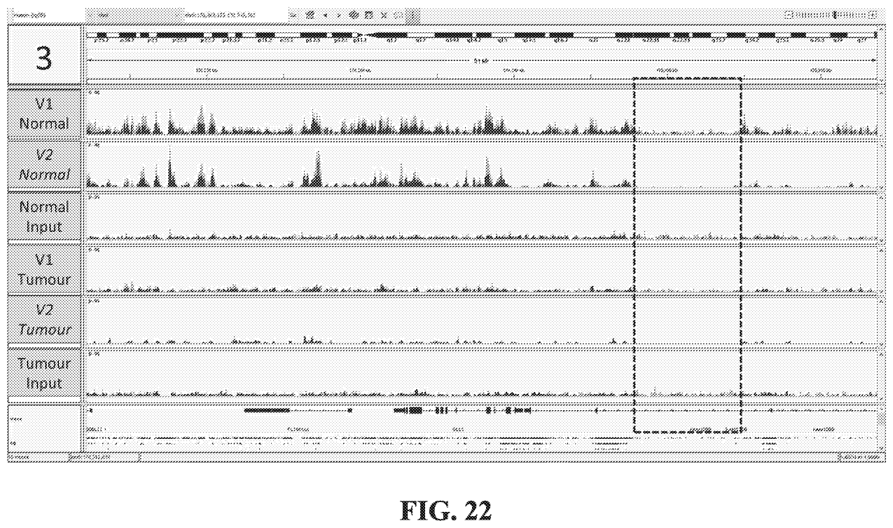

FIG. 22 shows an IGV screenshot comparing HMCP and CLE methods of the DLL1 gene and adjacent loci on chromosome 6. In the region bounded by the dotted line box, there are very few CpG motifs and hence very few reads may be expected in this region unless associated with focal hyper-hydroxymethylation. Whilst the CLE method shows virtually no reads in this region, the HMCP method has a scattering of reads that resemble ‘input’ levels of coverage.

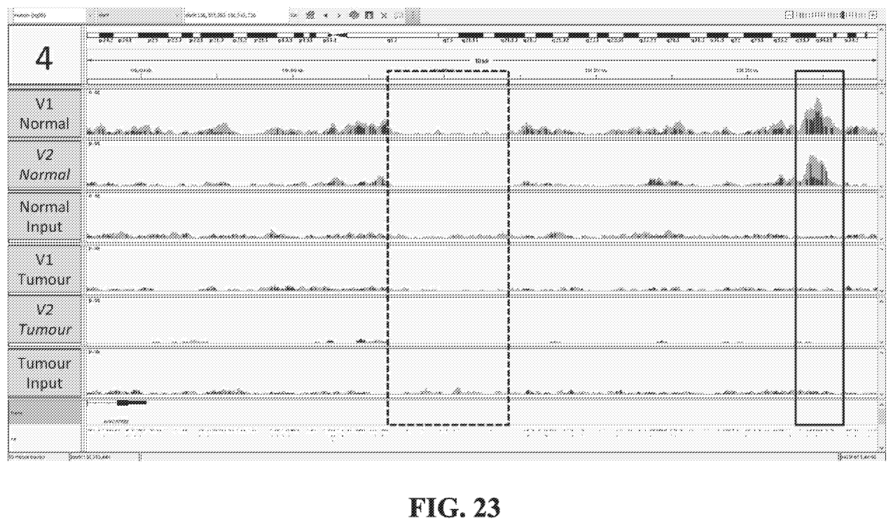

FIG. 23 shows an IGV screenshot comparing HMCP and CLE methods of a region on chromosome 9 at higher resolution. Here, a CpG-sparse region (dotted line box) may attract many fewer background reads when the CLE method is used compared with the HMCP method. The stronger 5-hmC peak in the solid line box is picked up by both methods.

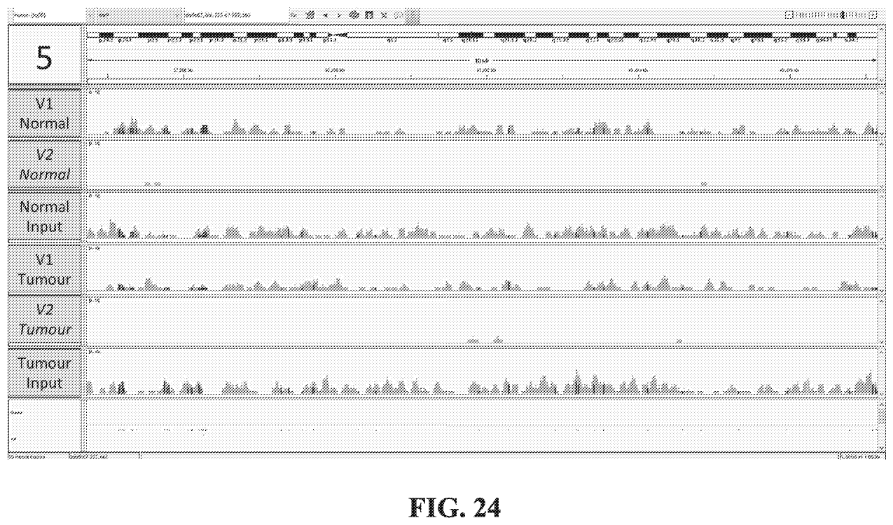

FIG. 24 shows an IGV screenshot comparing HMCP and CLE methods of another region of Chr9 with sparse CpG distribution. Again, the CLE method advantageously does not recover reads in this region unlike the HMCP method which gives ‘input-like’ coverage. In FIG. 24, the vertical axis is scaled down to 15 reads to highlight the low levels of coverage.

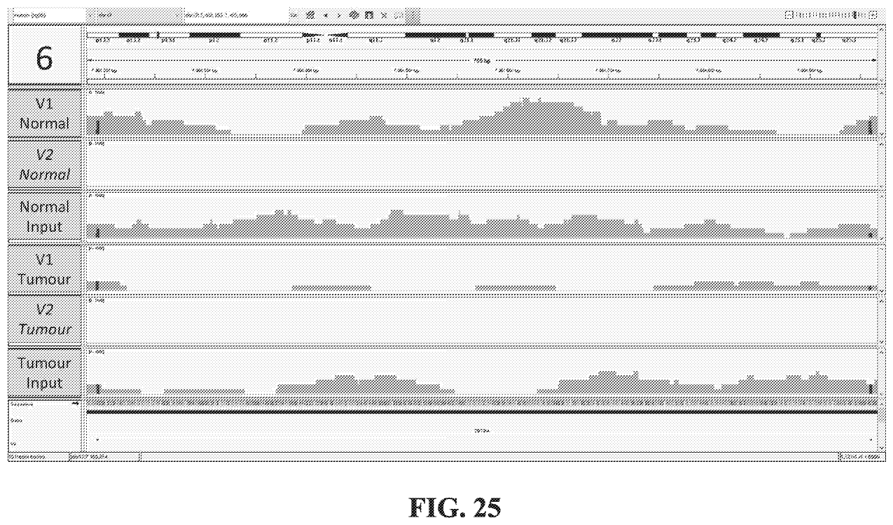

FIG. 25 shows an IGV screen shot comparing HMCP and CLE methods of a 785 bp region of human Chr17 where there is a large gap between CpG islands (˜750 base pair (bp); the CpG motifs are marked by the two dots in the bottom horizontal track (labelled ‘cg’)). The vertical scale is set from 0-10 in these panels. In this region, where reads may not be pulled down due to the absence of CpG motifs, the HMCP method pulls down reads with a similar level of coverage to the input sample whilst the CLE method advantageously pulls down substantially no reads on tumour or healthy tissue.

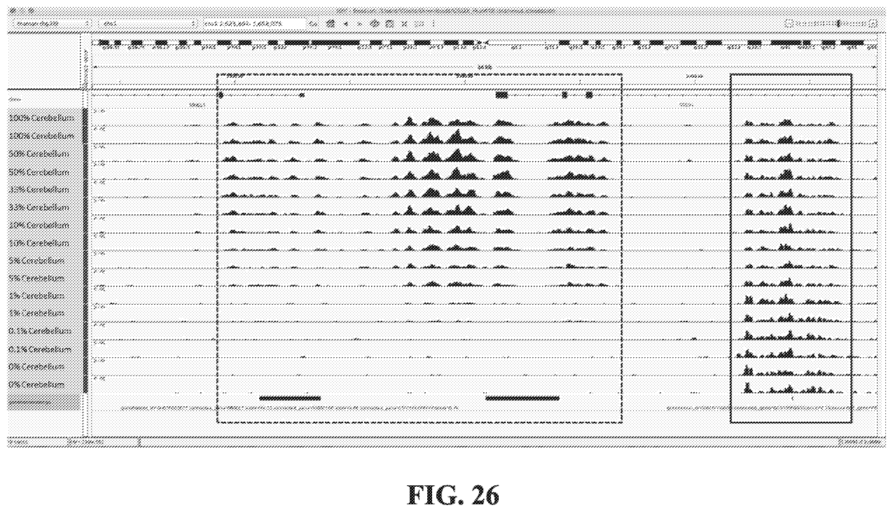

FIG. 26 shows an IGV screenshot of brain-specific 5-hmC peaks that can be detected in the context of the NA12878 derived peaks at levels as low as about 1% cerebellum. The CLE method advantageously may detect trace amounts of cerebellum DNA spiked into an unrelated, cell-line derived whole genomic DNA (wgDNA) sample (NA12878). A reference dataset is generated in duplicate using about 10 nanogram (ng) cerebellum DNA (100% cerebellum). Decreasing amounts of cerebellum DNA are spiked into about 10 ng NA12878 wgDNA and 5-hmC pulled down using the CLE method. The IGV screenshot of FIG. 26 shows brain-specific 5-hmC peaks (dotted line box) can be detected in the context of the NA12878 derived peaks (for example, those shown in the solid line box) at levels as low as about 1% cerebellum (100 picogram (pg) in 10 ng).

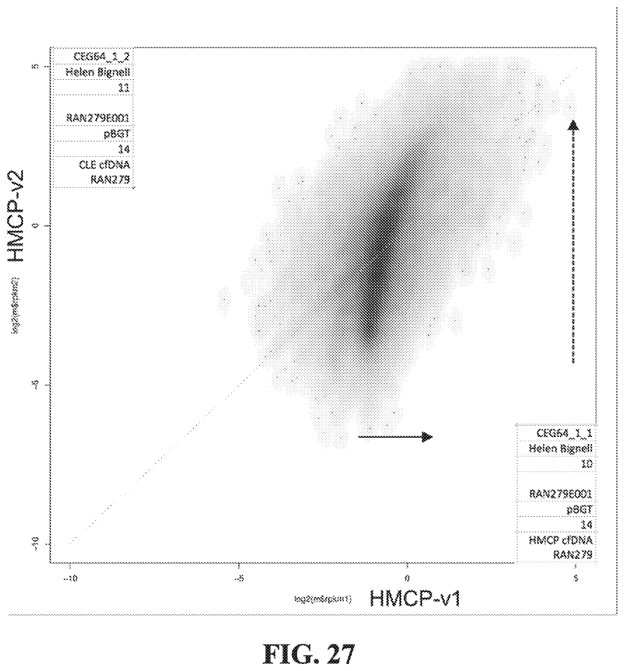

FIG. 27 shows a scatterplot comparison of HMCP and CLE methods using plasma DNA. A same biological sample may be analyzed with the standard HMCP method (RPKM1) or the CLE method (RPKM2). The CLE method may advantageously have a substantially wider amplitude (dotted line) than the standard HMCP method (black). FIG. 27 shows a smooth scatterplot where each point represents the log2 of the reads per kilobase per million mapped reads (RPKM) values for each gene in a pulldown experiment. The CLE method (y-axis) has a wider amplitude (broader dynamic range) as compared to the amplitude of the HMCP method (x-axis) over the same set of genes. The broader dynamic range in the CLE method may be an improvement over the narrower range of the HMCP method, a method that may be limited by background at the low end and/or signal saturation at the high-end.

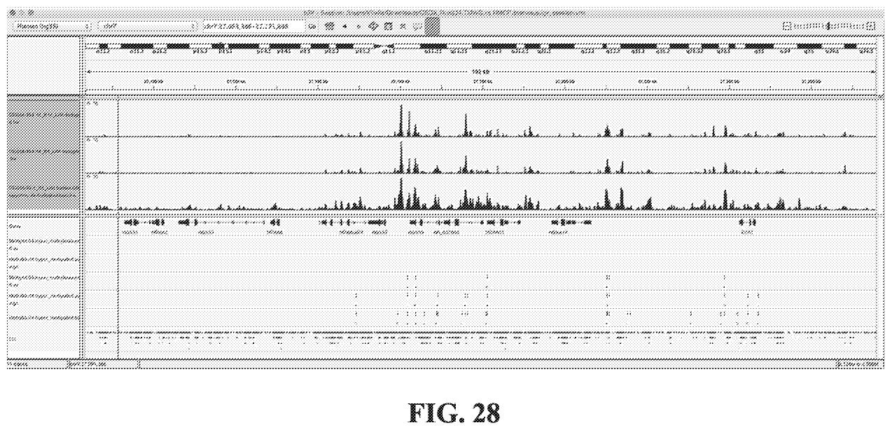

FIG. 28 shows an IGV screenshot highlighting a correlation between the HMCP method and TrueMethyl Whole Genome (TMWG) across different CpG densities. The IGV screenshot highlights a correlation between HMCP (top 3 tracks) and TrueMethyl Whole Genome (TMWG) over different CpG densities. The bottom panels in the 200 Kilobase (Kb) region of the human genome correspond to the HOX cluster of genes (bottom tracks). The CLE method (top two tracks) shows sharper and cleaner peaks than the HMCP method (third track). The highest CLE peaks correspond to the highest % 5-hmC from TrueMethyl WholeGenome (bottom tracks) and the peak high may be relatively lower for CpGs with lower % 5-hmC in TMWG.

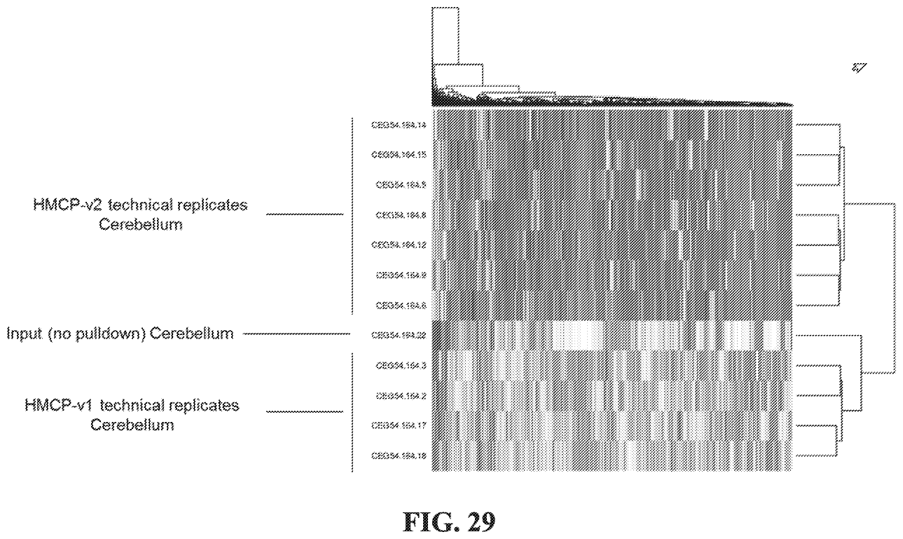

FIG. 29 shows a comparison of reads per kilobase per million mapped reads (RPKM) values on a heatmap between HMCP and CLE methods. RPKM values (adjusted read counts) for windows from about 80 to 100% 5-hmC in TrueMethyl Whole-Genome BS/oxBS MLML subtraction (cerebellum). The heatmap shows RPKM over different conditions (dark high RPKM values/bright low RPKM values). The CLE method has higher RPKMs than the HMCP method and to the input (no pulldown) null state for those regions corresponding to high 5-hmC in TrueMethyl Whole-Genome (cerebellum).

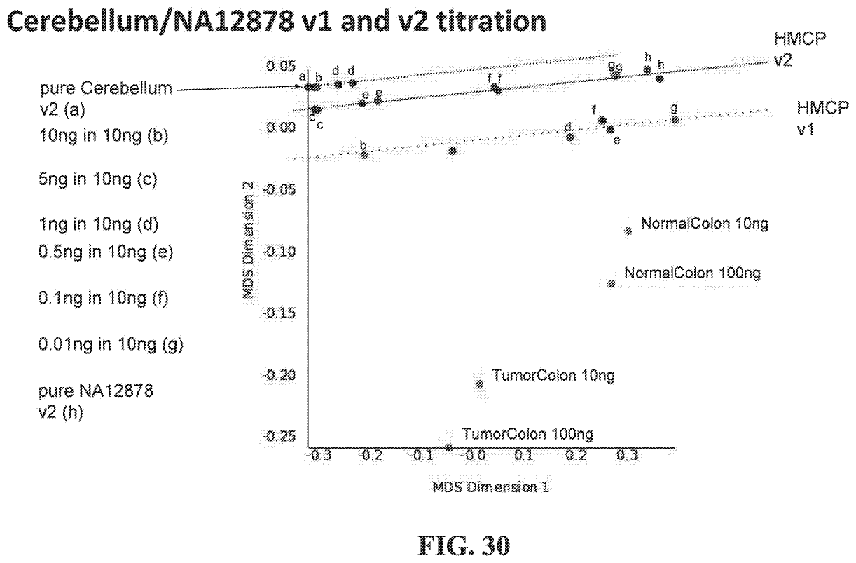

FIG. 30 shows a multidimensional scaling (MDS) plot. The MDS plot shows a level of similarity of read counts over genebodies for samples from a titration of cerebellum tissue genomic DNA (gDNA) into a background of NA12878 peripheral blood mononuclear cell (PBMC) cell line gDNA, as well as tissue normal colon and tumor colon samples with about 10 nanogram (ng) or about 100 ng of starting material. Titration of the CLE method (dense top and middle dotted lines) shows an exponential decay having similarity to the pure cerebellum samples (a) from about 10 ng (b) and about 5 ng (c) to about 1 ng (d) and about 0.5 ng (e), to about 0.1 ng (f) and about 0.01 ng (g), the latter being the closest to pure NA12878 (h). The same material utilized with the HMCP method shows less concordance in the similarity with the pure samples (bottom dotted line).The MDS Dimension 2 (y-axis) classifies samples by tissue, showing the colon samples separated from the cerebellum/NA12878 samples along the y-axis.

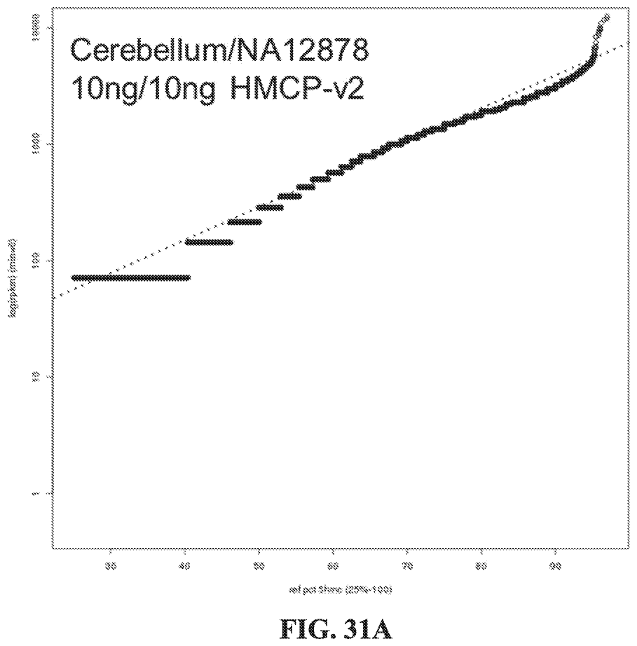

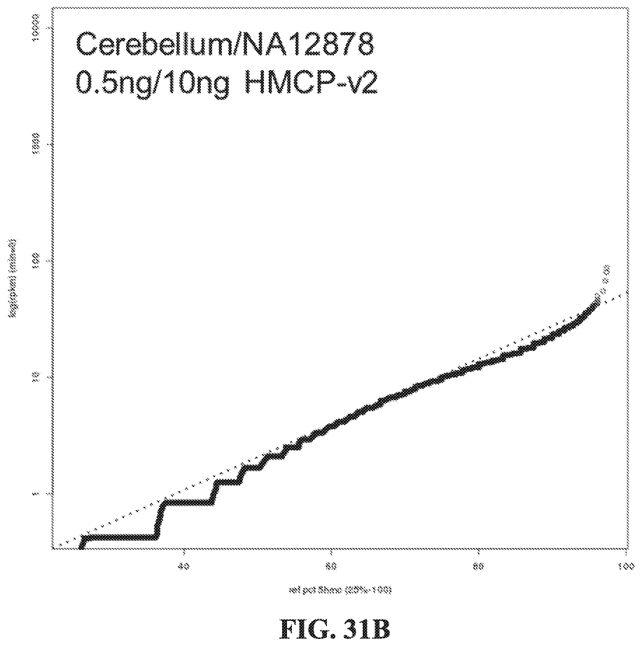

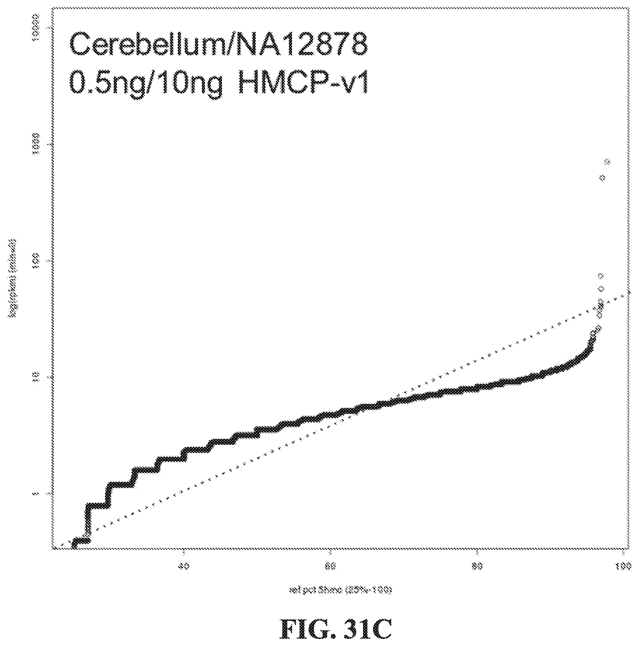

FIG. 31A-31C show Q-Q plots of TMWG % 5-hmC (25.01-99.99%) and HMCP genebodies RPKM. FIG. 31A shows the CLE method with high RPKM values (log scale) corresponding with increasing % 5-hmC in TMWG. FIG. 31B shows the CLE method with about 0.5 ng cerebellum demonstrating good correspondence with increasing % 5-hmC but by decreased overall RPKMs (log scale). FIG. 31C shows the HMCP method with about 0.5 ng cerebellum demonstrating flatter correspondence of RPKMs (log scale) to TMWG.

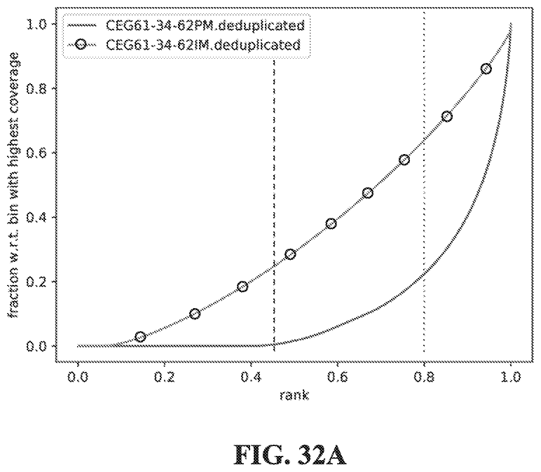

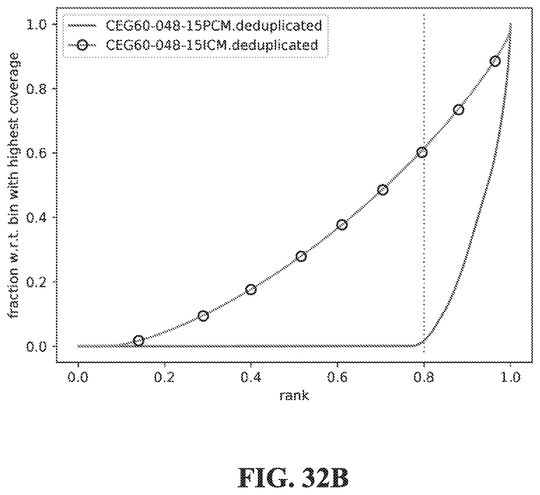

FIG. 32A-B show comparisons of sequence read enrichment for HMCP and CLE methods. The pulldown efficiency of the CLE method is advantageously higher than the HMCP method as determined by a higher fraction of the sequenced reads being enriched, as shown in FIG. 32A-B. The curved line shows the CLE method (FIG. 32B) concentrating the sequencing reads in about 20% of the genome (FIG. 32B, dotted line), as compared to the HMCP method (FIG. 32A) where a fraction of the reads are spent outside the enrichment (FIG. 32A, dashed to dotted line).

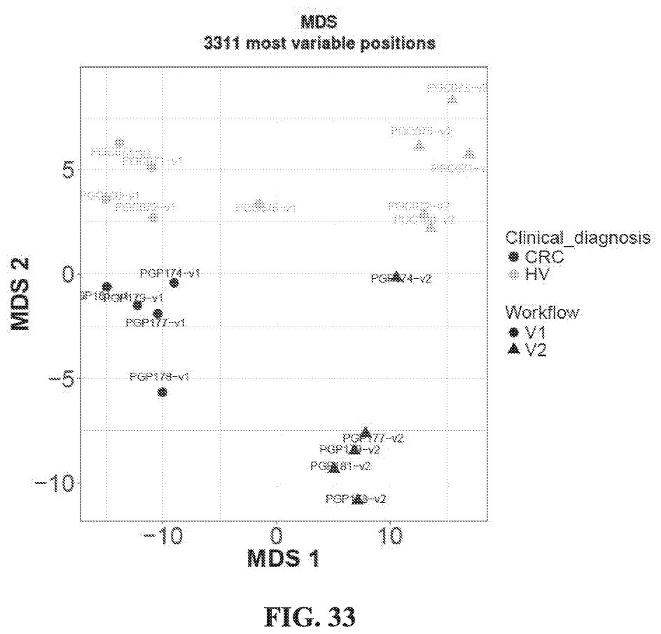

FIG. 33 shows a multidimensional scaling (MDS) plot for 3311 functional regions of the human genome. This figure demonstrates that the CLE method (v2, triangle) provides better separation of colorectal cancer cfDNA samples (CRC, black circle) from healthy volunteer cell free DNA (cfDNA) samples (HV, gray circle) as compared to the HMCP method (v1, circle). The MDS plot is done using 3311 functional regions of the human genome as annotated by the genehancer database (a composition of ENCODE, Ensembl, FANTOM and VISTA datasets).

An MDS plot may be a way of visualizing similarity between samples in a dataset, in a two dimensional space. Each point may represent one sample, which may be labelled by its clinical identifier and the method used (for example, HMCP or CLE method). The plot may be based on the RPKM ratio of pulldown:input of the top varying features. An euclidean distance may be calculated between samples, based on the variation in the data, represented as a distance matrix. This approach may be used to create coordinates of the points on the MDS plot. In some cases, two points that may be close to each other on an MDS plot may be more closely related in their RPKM enrichment profile than two points that may be distant each other.

While preferred embodiments have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Furthermore, it shall be understood that all aspects described herein are not limited to the specific depictions, configurations or relative proportions set forth herein which depend upon a variety of conditions and variables. It should be understood that various alternatives to the embodiments described herein may be employed. It is therefore contemplated that the disclosure shall also cover any such alternatives, modifications, variations or equivalents. It is intended that the following claims define the scope of the disclosure and that methods and structures within the scope of these claims and their equivalents be covered thereby.

What is claimed is:

1. A method comprising:
a. associating a label with an epigenetically modified base of a nucleic acid sequence to form a labeled nucleic acid sequence;
b. hybridizing a substantially complementary strand to the labeled nucleic acid sequence; and
c. amplifying the substantially complementary strand in a reaction in which the labeled nucleic acid sequence is substantially not present.

2. A method comprising:
a. hybridizing a substantially complementary strand to a nucleic acid sequence comprising an epigenetically modified base;
b. associating a label with the epigenetically modified base of a nucleic acid sequence to form a labeled nucleic acid sequence; and
c. amplifying the substantially complementary strand in a reaction in which the labeled nucleic acid sequence is substantially not present.

3. The method of any one of claims 1-2, wherein the label is associated with a substrate.

4. The method of claim 3, wherein the substrate comprises a bead.

5. The method of claim 4, wherein the bead is a magnetic bead.

6. The method of claim 3, wherein the substrate comprises an array.

7. The method of any one of claims 1-6, wherein the substantially complimentary strand is shorter in length than the labeled nucleic acid sequence.

8. The method of any one of claims 1-7, wherein the substantially complimentary strand is elongated before the amplifying.

9. The method of any one of claims 1-8, wherein hybridizing comprises hybridizing at least two substantially complementary strands to the labeled nucleic acid sequence.

10. The method of claim 9, comprising ligating the at least two substantially complementary strands.

11. The method of any one of claims 1-10, wherein the labeled nucleic acid sequence comprises an adapter sequence.

12. The method of claim 11, wherein hybridizing comprises hybridizing at least a portion of the substantially complimentary strand to the adapter sequence.

13. The method of any one of claims 1-12, wherein the nucleic acid sequence comprises a first barcode.

14. The method of any one of claims 1-13, wherein the nucleic acid sequence comprises a second barcode.

15. The method of claim 14, wherein the first barcode is a unique barcode and the second barcode is a sample barcode.

16. The method of any one of claims 1-15, wherein the epigenetically modified base of the nucleic acid sequence is a hydroxymethylated base (hmB).

17. The method of claim 16, wherein the hmB is 5-hydroxymethylated base (5-hmB).

18. The method of claim 17, wherein the 5-hmB is a 5-hydroxymethylated cytosine (5-hmC).

19. The method of any one of claims 1-15, wherein the epigenetically modified base of the nucleic acid sequence comprises a methylated base, a hydroxymethylated base, a formylated base, or a carboxylic acid containing base or a salt thereof.

20. The method of any one of claims 1-19, wherein at least a portion of the nucleic acid sequence or the labeled nucleic acid sequence is double-stranded.

21. The method of any one of claims 1-20, wherein the label is associated with the epigenetically modified base by a single bond, a double bond, or a triple bond.

22. The method of any one of claims 1-21, comprising separating the substantially complementary strand from the labeled nucleic acid sequence.

23. The method of any one of claims 1-22 , wherein the nucleic acid sequence comprises at least: from about 1 to about 3; from about 1 to about 5; from about 1 to about 10; from about 1 to about 15; or from about 1 to about 20 epigenetically modified bases per at least about 20 bases of the nucleic acid sequence.

24. The method of any one of claims 1-23, wherein the nucleic acid sequence comprises at least about: 1, 5, 10, 15 or 20 epigenetically modified bases per at least about 20 bases of the nucleic acid sequence.

25. The method of any one of claims 1-24, wherein at least about: 70%, 75%, 80%, 85%, 90%, or 95% of bases of the substantially complementary strand base pair with the labeled nucleic acid sequence.

26. The method of any one of claim 1-25, wherein the substantially complementary strand hybridizes to the nucleic acid sequence under stringent hybridization conditions.

27. The method of any one of claims 1-25, wherein the nucleic acid sequence comprises a cytosine guanine (CG) site, a cytosine phosphate guanine (CpG) island, or a combination thereof.

28. The method of any one of claims 1-27, wherein the nucleic acid sequence comprises cell-free DNA.

29. The method of any one of claims 1-28, wherein the nucleic acid sequence comprises a cDNA sequence.

30. The method of any one of claims 1-29, comprising sequencing an amplified product.

31. The method of any one of claims 1-30, wherein the nucleic acid sequence is from a sample.

32. The method of claim 31, wherein the sample is from a subject.

33. The method of claim 32, wherein the subject is a human.

34. The method of any one of claims 31-33, wherein the sample comprises a buccal sample, a saliva sample, a blood sample, a plasma sample, a reproductive sample, a mucus sample, cerebral spinal fluid sample, a tissue sample, or any combination thereof.

35. The method of claim 32-34, comprising obtaining a result.

36. The method of claim 35, comprising comparing the result to a reference.

37. The method of claim 35 or 36, comprising communicating the result via a communication medium.

38. The method of any one of claims 32-37, wherein the subject is diagnosed with a condition.

39. The method of any one of claims 32-37, comprising diagnosing the subject as having a condition.

40. The method of any one of claims 32-37, comprising diagnosing the subject as having a likelihood of developing a condition.

41. The method of claim 39 or 40, wherein the diagnosing is based on the comparing the result to the reference.

42. The method of any one of claim 38-39, wherein the diagnosing at least partially confirms a previous diagnosis.

43. The method of claim 39, wherein the condition is a cancer.

44. The method of claim 39 or 43, comprising selecting a treatment for the subject.

45. The method of any one of claims 39-44, comprising treating the subject.

46. The method of claim 45, wherein the treating comprises: surgery, chemotherapy, radiation therapy, immunotherapy, targeted therapy, hormone therapy, stem cell transplant, and precision medicine.

47. The method of any one of claims 1-37, comprising repeating the associating, the hybridizing and the amplifying at different time points.

48. The method of claim 32, wherein the subject is a human.

49. The method of any one of claims 1-48, wherein the label comprises a sugar.

50. The method of claim 49, wherein the sugar comprises a glucose.

51. The method of claim 50, wherein the glucose is modified.

52. The method of any one of claims 1-51, wherein the label is associated with the epigenetically modified base with the assistance of an enzyme.

53. The method of claim 52, wherein the enzyme is selective for a portion of the nucleic acid sequence that is double-stranded.

54. The method of any one of claims 1-52, wherein the label is selectively associated with the epigenetically modified base at a portion of the nucleic acid sequence that is double-stranded.

55. The method of any one of claims 1-52, wherein the label is selective for a portion of the nucleic acid sequence.

56. The method of claim 54, wherein the portion is double-stranded.

57. The method of any one of claims 1-56, wherein the substantially complementary strand is substantially free of an epigenetically modified base.

58. The method of any one of claims 1-56, wherein the substantially complementary strand is free of an epigenetically modified base.

59. The method of any one of claims 1-56, wherein the amplifying results in a plurality of nucleic acid strands, wherein less than about 2% of the plurality of nucleic acid strands comprise an epigenetically modified base.

60. The method of any one of claims 1-56, wherein the nucleic acid sequence comprises a plurality of epigenetically modified bases, and wherein the substantially complementary strand comprises less than about 2% of the plurality of epigenetically modified bases.

61. The method of any one of claims 1-56, wherein the substantially complementary strand comprises an epigenetically modified base.

62. A kit comprising: instructions for use; a container; a label configured to (i) associate with an epigenetically modified nucleic acid sequence and to (ii) associate with a substrate; a control nucleic acid sequence associated with a substrate and a substrate configured to associate with the label.

63. A method comprising: detecting a presence of a plurality of epigenetically modified residues in a nucleic acid sequence, wherein the plurality of epigenetically modified residues comprises at least 2 epigenetically modified residues, and wherein a sensitivity of detection remains substantially constant with an increasing number of epigenetically modified residues in the plurality of epigenetically modified residues.

64. The method of claim 63, wherein the at least 2 epigenetically modified residues is at least 4 epigenetically modified residues.

65. The method of claim 63, wherein the sensitivity of detection comprises detecting a presence of at least about 90% of the plurality of epigenetically modified residues.

66. The method of claim 65, wherein the sensitivity of detection comprises detecting a presence of each epigenetically modified residue of the plurality of epigenetically modified residues.

67. A method comprising: enriching a nucleic acid sequence, wherein the nucleic acid sequence comprises (i) a plurality of epigenetically modified residues and (ii) a sequence length, wherein the plurality of epigenetically modified residues comprises at least 2 epigenetically modified residues, wherein the enriching comprises at least 4 cycles of amplification and produces a plurality of sequence reads, and wherein about 90% of the plurality of sequence reads retain at least about 90% of the sequence length.

68. The method of claim 67, wherein the at least 2 epigenetically modified residues is at least 4 epigenetically modified residues.

69. The method of claim 67, wherein the at least 4 cycles of amplification is at least 8 cycles of amplification.

70. The method of any one of claims 63-69, wherein the nucleic acid sequence comprises cell-free DNA.

71. The method of any one of claims 63-69, wherein the nucleic acid sequence comprises a cDNA sequence.

72. The method of any one of claims 63-69, wherein an epigenetically modified residue of the plurality of epigenetically modified residues is a hydroxymethylated base (hmB).

73. The method of claim 72, wherein the hmB is 5-hydromethylated base (5-hmB).

74. The method of claim 73, wherein the 5-hmB is a 5-hydroxymethylated cytosine (5-hmC).

75. The method of any one of claims 63-69, wherein an epigenetically modified residue of the plurality of epigenetically modified residues comprises a methylated base, a hydroxymethylated base, a formylated base, or a carboxylic acid containing base or a salt thereof.

76. The method of any one of claims 63-75, wherein at least a portion of the nucleic acid sequence is double-stranded.

77. The method of any one of claims 63-75, wherein the nucleic acid sequence comprises a cytosine guanine (CG) site, a cytosine phosphate guanine (CpG) island, or a combination thereof.

78. A method comprising: enriching a nucleic acid sequence comprising a plurality of epigenetically modified residues to produce a plurality of sequence reads, wherein at least about 90% of the plurality of sequencing reads produced from the enriching are from about 1% to about 50% of a genome.

79. The method of claim 78, wherein the at least about 90% of the plurality of sequencing reads produced are from about 1% to about 20% of the genome.

80. The method of claim 78, wherein a length of the plurality of sequencing reads is at least about 10 basepairs.

81. The method of claim 78, wherein the plurality of epigenetically modified residues is at least about 2 epigenetically modified residues.

82. The method of claim 81, wherein the plurality of epigenetically modified residues is at least about 6 epigenetically modified residues.

83. The method of any one of claims 63-82, wherein a label is associated with an epigenetically modified residue of the plurality of epigenetically modified residues.

84. The method of claim 83, wherein the label is associated with the epigenetically modified residue by a single bond, a double bond, or a triple bond.

85. The method of any one of claims 63-84, wherein the nucleic acid sequence comprises at least: from about 1 to about 3; from about 1 to about 5; from about 1 to about 10; from about 1 to about 15; or from about 1 to about 20 epigenetically modified residues per at least about 20 bases of the nucleic acid sequence.

86. The method of any one of claims 63-85, wherein the nucleic acid sequence comprises at least about: 1, 5, 10, 15 or 20 epigenetically modified residues per at least about 20 bases of the nucleic acid sequence.

87. The method of any one of claims 78-86, wherein the nucleic acid sequence comprises cell-free DNA.

88. The method of any one of claims 78-87, wherein the nucleic acid sequence comprises a cDNA sequence.

89. The method of any one of claims 63-88, wherein the nucleic acid sequence is from a sample.

90. The method of claim 89, wherein the sample is obtained from a subject.

91. The method of claim 90, wherein the subject is a human.

92. The method of any one of claims 90-91, wherein the sample comprises a buccal sample, a saliva sample, a blood sample, a plasma sample, a reproductive sample, a mucus sample, cerebral spinal fluid sample, a tissue sample, or any combination thereof.

93. The method of any one of claims 63-92, further comprising obtaining a result.

94. The method of claim 93, further comprising comparing the result to a reference.

95. The method of claim 93 or 94, further comprising communicating the result via a communication medium.

96. The method of any one of claims 94-95, wherein the subject is diagnosed with a condition.

97. The method of any one of claims 94-95, further comprising diagnosing the subject as having a condition.

98. The method of any one of claims 94-95, further comprising diagnosing the subject as having a likelihood of developing a condition.

99. The method of claim 97 or 98, wherein the diagnosing is based on the comparing the result to the reference.

100. The method of any one of claims 98-99, wherein the diagnosing at least partially confirms a previous diagnosis.

101. The method of claim 96, wherein the condition is a cancer.

102. The method of claim 96 or 101, further comprising selecting a treatment for the subject.

103. The method of any one of claims 97-102, further comprising treating the subject.

104. The method of claim 103, wherein the treating comprises: surgery, chemotherapy, radiation therapy, immunotherapy, targeted therapy, hormone therapy, stem cell transplant, and precision medicine.

105. The method of any one of claims 83-84, wherein the label comprises a sugar.

106. The method of claim 105, wherein the sugar comprises a glucose.

107. The method of claim 106, wherein the glucose is modified.

108. The method of any one of claims 83-84, wherein the label is associated with the epigenetically modified residue with assistance of an enzyme.

109. The method of claim 108, wherein the enzyme is selective for a portion of the nucleic acid sequence that is double-stranded.

110. The method of any one of claims 83-84, wherein the label is selectively associated with the epigenetically modified residue at a portion of the nucleic acid sequence that is double-stranded.

111. The method of any one of claims 83-84, wherein the label is selective for a portion of the nucleic acid sequence.

112. The method of claim 111, wherein the portion is double-stranded.

113. A method for identifying a cell-free sample as benign or malignant for a cancer, the method comprising: assaying the cell-free sample by next generation sequencing to identify a nucleic acid sequence, wherein a presence of a 5-hydroxymethylcytosine (5-hmC) in the nucleic acid sequence identifies the cell-free sample as malignant for the cancer.

114. The method of claim 113, wherein the cell-free sample is obtained from a subject having or suspected of having said cancer.

115. The method of claim 114, further comprising selecting a treatment for the subject based on the presence of the 5-hmC.

116. The method of claim 113, wherein the presence of the 5-hmC comprises a level of 5-hmC in the cell-free sample.

117. The method of claim 113, wherein the nucleic acid sequence comprises a cytosine guanine (CG) site, a cytosine phosphate guanine (CpG) island, or a combination thereof.

118. The method of claim 113, further comprising obtaining a result based on the presence of the 5-hmC.

119. The method of claim 118, further comprising communicating the result via a communication medium.

120. The method of claim 113, wherein a label is associated with an epigenetically modified base of the nucleic acid sequence.
